SEQUENCE LISTING (65 sequences)

SEQ ID NO: 1 - DNA, 4596 bp, Artificial Sequence
  Synthetic Sequences
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttctaatgaagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctttcctggactacttcagacgaacttcgtagggcgcataagtctcgaccacgcaatgacgcagcgatgcttgaaaaaaacaccgctttcaaggcggctggacgaagaccgcaagacaccctccacctcacctagcctgtatctctgcaatagcctaattacttcggaatctcctgtcgtaattccttagataaacggcaattaggtgacactctaaatctgtgtggaaccggcttccaaacactctaccacctctattagtgacacagagagaatccttgagtggcttctaggtatattgaacaacttcatcacgaattgagcagtgatcatggttctacgtatcaaccaatattaaccactgtgctctgtagcattgctaaatcgggctgtctgtttcaccatagatcgtgaggccatgcccacgggcattagaacttagcctgtttagcgataatcccaacaatgagctgggatatgacgagaagtatttagcaaccttttcgtgatcggctacgtaaaacctcatattacggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 4790 bp, Artificial Sequence
  Synthetic Sequences
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggcgcgcccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttctaatgaagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctttcctggactacttcagacgaacttcgtagggcgcataagtctcgaccacgcaatgacgcagcgatgcttgaaaaaaaccccgctttcaaggcggctggacgaagaccgcaagacaccctccacctcacctagcctgtatctctgcaatagcctaattacttcggaatctcctgtcgtaattccttagataaacggcaattaggtgacactctaaatctgtgtggaaccggcttccaaacactctaccacctctattagtgacacagagagaatccttgagtggcttctaggtatattgaacaacttcatcacgaattgagcagtgatcatggttctacgtatcaaccaatattaaccactgtgctctgtagcattgctaaatcgggctgtctgtttcaccatagatcgtgaggccatgcccacgggcattagaacttagcctgtttagcgataatcccaacaatgagctgggatatgacgagaagtatttagcaaccttttcgtgatcggctacgtaaaacctcatattacggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 3 - DNA, 4700 bp, Artificial Sequence
  Synthetic Sequences
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccagtggcaaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttctaatgaagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctttcctgtcgacggaggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - DNA, 4531 bp, Artificial Sequence
  Synthetic Sequences
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttcggatccggcgaaggcagaggctcactgcttacttgtggcgacgtggaggagaaccccggacctatggtttccaagggcgaagaactgtttaccggcgtggtgcccatcctggtggaactggatggcgacgttaacggacacaagttcagcgtcagcggagaaggcgaaggcgacgccacatacggaaagctgacactgaagtttatctgcaccaccggcaagctgcccgtgccttggcctacactggtcaccacactgacatacggcgtgcagtgcttcagcagataccccgaccacatgaagcagcacgatttcttcaagagcgccatgcctgagggctacgtgcaagagcggaccatcttcttcaaggacgacgggaactacaagaccagagccgaagtgaagttcgagggcgacaccctcgtgaaccggatcgagctgaagggcatcgacttcaaagaggacggaaacatcctgggccacaaacttgagtacaactacaacagccacaacgtctacatcatggccgacaagcagaaaaacggcatcaaagtgaacttcaagatccggcacaacatcgaggacggctctgtgcagctggctgaccactaccagcagaacacacccatcggagatggccctgtgctgctgcccgataaccactacctgagcacacagagcgccctgagcaaggaccccaacgagaagagggatcacatggtgctgctggaatttgtgaccgctgccggcatcaccctcggcatggatgaactgtacaagggctccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgatctagagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 5 - DNA, 4725 bp, Artificial Sequence
  Synthetic Sequences
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggcgcgcccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttcggatccggcgaaggcagaggctcactgcttacttgtggcgacgtggaggagaaccccggacctatggtttccaagggcgaagaactgtttaccggcgtggtgcccatcctggtggaactggatggcgacgttaacggacacaagttcagcgtcagcggagaaggcgaaggcgacgccacatacggaaagctgacactgaagtttatctgcaccaccggcaagctgcccgtgccttggcctacactggtcaccacactgacatacggcgtgcagtgcttcagcagataccccgaccacatgaagcagcacgatttcttcaagagcgccatgcctgagggctacgtgcaagagcggaccatcttcttcaaggacgacgggaactacaagaccagagccgaagtgaagttcgagggcgacaccctcgtgaaccggatcgagctgaagggcatcgacttcaaagaggacggaaacatcctgggccacaaacttgagtacaactacaacagccacaacgtctacatcatggccgacaagcagaaaaacggcatcaaagtgaacttcaagatccggcacaacatcgaggacggctctgtgcagctggctgaccactaccagcagaacacacccatcggagatggccctgtgctgctgcccgataaccactacctgagcacacagagcgccctgagcaaggaccccaacgagaagagggatcacatggtgctgctggaatttgtgaccgctgccggcatcaccctcggcatggatgaactgtacaagggctccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgatctagagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 6 - DNA, 4363 bp, Artificial Sequence
  Synthetic Sequences
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttcggatccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgatctagagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 7 - DNA, 4651 bp, Artificial Sequence
  Synthetic Sequences
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatggcgcgcctccagatatcatacactcgagcaactttgtatagaaaagttggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaagaattcacgcgtcaattgctcgaggccaccatggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttctgatgagatgcattcgaagcggccgcgagctcaagcttgcaattccgataacttgtttattgcagcttataatggttacaaataaagcaatagcatcacaaatttcacaaataaagcatttttttcactgcattctagttgtggtttgtccaaactcatcaatgtatcttatcatgtctggcgctagcagcacaagtttgtacaaaaaagcaggctcctcgcaggaggcctggccggcctccggattacttccggtgcaggcgtcagctgcatcttgctggccagctcggaggactgtccccgtaactcccgtccgctgtgtatcaacacagctctagccaaattgttgaccgaacgacggggttcgattgtatcattcctcacatccaacacttgagtagtacaatgccttgtctaagtgcgcttgggacccagtctattttgtttgtgtgcacaagacgatactcctcgatcacgtaccaagcgcaggctggccctaacgacgtggtattctcgtattggtgtgccccagattgcataaggcactcatcacctgcgtttatgacgctggacgcctgcgtatgttgcaggactcctatgatgtcgctagcctgatgcgttgttatgtgattgaggtttagtacttggcaatatcattagatggaactaatacgcggtctaatcgttacaggtttccgacgattgagaagtatcacctgattctcgggagcgacttgtaactctactacacttattgatttggtactcctatgtgttgctatcttgactccgtaaatcgccgtgatacactctcttcgtttctcggcaatctatggctctaaactatacacaggcattctaagaataatctgacctgtccctgtagtactggtaggttactaccagtacggcgacaatgtgggagctacggcgtggtagaaccgggtacagacacagtccctgatcccgatagtaacactgtgcccgtagcagtgataatcattgcgcctcaacctgaggaagataagggaggcagtatatcgtgctattccctctattctcccacatgcgtatggcctgaggtcaatgatcaggtacatgccgtaatatgaggttttacgtagccgatcacgaaaaggttgctaaatacttctcgtcatatcccagctcattgttgggattatcgctaaacaggctaagttctaatgcccgtgggcatggcctcacgatctatggtgaaacagacagcccgatttagcaatgctacagagcacagtggttaatattggttgatacgtagaaccatgatcactgctcaattcgtgatgaagttgttcaatatacctagaagccactcaaggattctctctgtgtcactaatagaggtggtagagtgtttggaagccggttccacacagatttagagtgtcacctaattgccgtttatctaaggaattacgacaggagattccgaagtaattaggctattgcagagatacaggctaggtgaggtggagggtgtcttgcggtcttcgtccagccgccttgaaagcggggtttttttcaagcatcgctgcgtcattgcgtggtcgagacttatgcgccctacgaagttcgtctgaagtagtccaggaaagacctactttgcagttatcttcgcattcccacactcaccactacaactactcttccctcaatttcccggttagtttcgctaagctccgaccttgggttactgtgttgcatccgactcgctgcggctttctagtacgctgtactgtttcattcttctgtaggtctggttccgtaagtccgaatttccaggccgtggtctagtcctaattattttctgtcccggtagctatatttagccgagggtttgtccatttgcccggcgtagagcgccgcgtttgcgaacatttgcgcccgtaatacgtagggacaccgtcgggtaatggatggcaaaagccgaaaacggcgtcttccggcgcttggattcagcgctcttgagccataaaccgcgttgcttctttggttaattcgtattaatgatcctaagcgccagcttattcgttaagaggcactaggcgcgccgcggcatgcgatcgccagcatggctacgacccagctttcttgtacaaagtggtgatggccggccgcttcgagttaattaatccaaccggttaccgcctaggatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 8 - DNA, 6724 bp, Artificial Sequence
  Synthetic Sequences
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattaaccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccgcggtctagaaagaggtaagggtttaagggatggttggttggtggggtattaatgtttaattacctggagcacctgcctgaaatcactttttttcaggttggacgcgtcgctagcatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 9 - DNA, 6550 bp, Artificial Sequence
  Synthetic Sequences
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 10 - DNA, 6550 bp, Artificial Sequence
  Synthetic Sequences
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgctagcatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 11 - DNA, 6172 bp, Artificial Sequence
  Synthetic Sequences
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaagaattctaatacgacgtctctgacctaggccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 12 - DNA, 6234 bp, Artificial Sequence
  Synthetic Sequences
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcctaggccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 13 - DNA, 6748 bp, Artificial Sequence
  Synthetic Sequences
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaagaattctaatacgacgtctctgacctaggccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtacggctccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 14 - DNA, 6810 bp, Artificial Sequence
  Synthetic Sequences
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcctaggccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtacggctccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 15 - DNA, 3246 bp, Artificial Sequence
  Synthetic Sequences
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcctaggccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcacctgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 16 - DNA, 3184 bp, Artificial Sequence
  Synthetic Sequences
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaagaattctaatacgacgtctctgacctaggccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcacctgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 17 - DNA, 3760 bp, Artificial Sequence
  Synthetic Sequences
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaagaattctaatacgacgtctctgacctaggccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcaccggctccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 18 - DNA, 3822 bp, Artificial Sequence
  Synthetic Sequences
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcctaggccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcaccggctccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 19 - DNA, 4582 bp, Artificial Sequence
  Synthetic Sequences
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcctaggccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcacctaatgaagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctttcctggactacttcagacgaacttcgtagggcgcataagtctcgaccacgcaatgacgcagcgatgcttgaaaaaaacaccgctttcaaggcggctggacgaagaccgcaagacaccctccacctcacctagcctgtatctctgcaatagcctaattacttcggaatctcctgtcgtaattccttagataaacggcaattaggtgacactctaaatctgtgtggaaccggcttccaaacactctaccacctctattagtgacacagagagaatccttgagtggcttctaggtatattgaacaacttcatcacgaattgagcagtgatcatggttctacgtatcaaccaatattaaccactgtgctctgtagcattgctaaatcgggctgtctgtttcaccatagatcgtgaggccatgcccacgggcattagaacttagcctgtttagcgataatcccaacaatgagctgggatatgacgagaagtatttagcaaccttttcgtgatcggctacgtaaaacctcatattacggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtcacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 4137 bp, Artificial Sequence
  Synthetic Sequences
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcctaggccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcaccggatccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgatctagagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 21 - protein, 1457 aa, Artificial Sequence
  Synthetic Sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 22 - DNA, 4371 bp, Artificial Sequence
  Synthetic Sequence
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtac

SEQ ID NO: 23 - DNA, 232 bp, Artificial Sequence
  Synthetic Sequence
gagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaa

SEQ ID NO: 24 - DNA, 231 bp, Artificial Sequence
  Synthetic Sequence
ttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccct

SEQ ID NO: 25 - DNA, 136 bp, Artificial Sequence
  Synthetic Sequence
ctggtactgcatgcacgcaatgctagctgcccctttcccgtcctgggcaccccgagtctcccccgaccccgggtcccaggtatgctcccacctccacctgccccactcaccacctctgctagttccagacacctcc

SEQ ID NO: 26 - DNA, 139 bp, Artificial Sequence
  Synthetic Sequence
caagcacgcagcaatgcagctcaaaacgcttagcctagccacacccccacgggaaacagcagtgattaacctttagcaataaacgaaagtttaactaagctatactaaccccagggttggtcaatttcgtgccagccac

SEQ ID NO: 27 - DNA, 278 bp, Artificial Sequence
  Synthetic Sequence
ctggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 28 - DNA, 6828 bp, Artificial Sequence
  Synthetic Sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 29 - RNA, 100 nt, Artificial Sequence
  Synthetic Sequence
ccugggcaccagcgccggugguuuuagagcuagaaauagcaaguuaaaauaaggcuaguccguuaucaacuugaaaaaguggcaccgagucggugcuuuu

SEQ ID NO: 30 - RNA, 100 nt, Artificial Sequence
  Synthetic Sequence
gaugcucgcccucccgucccguuuuagagcuagaaauagcaaguuaaaauaaggcuaguccguuaucaacuugaaaaaguggcaccgagucggugcuuuu

SEQ ID NO: 31 - protein, 1591 aa, Artificial Sequence
  Synthetic Sequence
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 32 - protein, 199 aa, Artificial Sequence
  Synthetic Sequence
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 33 - protein, 1369 aa, Artificial Sequence
  Synthetic Sequence
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 34 - DNA, 7336 bp, Artificial Sequence
  Synthetic Sequence
tgtacatagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctggtctcatgacgcgtagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggta

SEQ ID NO: 35 - DNA, 7058 bp, Artificial Sequence
  Synthetic Sequence
tgtacatagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctggtctcatgacgcgtagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggta

SEQ ID NO: 36 - DNA, 4374 bp, Artificial Sequence
  Synthetic Sequence
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 37 - protein, 593 aa, Artificial Sequence
  Synthetic Sequence
GSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 38 - protein, 605 aa, Artificial Sequence
  Synthetic Sequence
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 39 - protein, 604 aa, Artificial Sequence
  Synthetic Sequence
APKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 40 - DNA, 2250 bp, Artificial Sequence
  Synthetic Sequence
atgctgcgggccaagaatcaactgttcctgctgtcccctcactacctgcggcaagtgaaagagagcagcggcagcagactgatccagcagagactgctgcatcagcagcagccactgcaccctgaatgggccgctctggctaagaagcagctcaagggcaagaaccccgaggacctgatctggcacacaccagagggcatcagcatcaagcccctgtactccaagcgggacacaatggatctgcccgaggaactgcctggcgtgaagccttttacaagaggcccctatcctaccatgtataccttcagaccctggaccatccggcagtacgccggcttttctaccgtggaagagagcaacaagttctacaaggacaacatcaaggccggccagcagggactgagcgtggcatttgatctggctacccacaggggctacgacagcgacaaccctagagtgcggggagatgttggaatggccggcgtggcaatcgacacagtggaagataccaagatcctgttcgacggcatccctctggaaaagatgagcgtgtccatgaccatgaacggcgctgtgatccccgtgctggctaactttattgtgaccggcgaggaacagggcgtgcccaaagaaaagctgaccggcaccatccagaacgacatcctgaaagagttcatggttcgaaacacctacatcttcccacctgagccgagcatgaagatcattgccgacatcttcgagtacaccgccaagcacatgcccaagttcaacagcatctccatcagcggctaccacatgcaagaggctggcgccgatgccatcctggaactggcttatacactggccgacggcctggaatactccagaacaggactgcaagccggcctgaccatcgatgagtttgcccctagactgagcttcttctggggcatcggcatgaacttctacatggaaatcgccaagatgagagccggcagacggctgtgggctcacctgatcgagaagatgttccagcctaagaacagcaagagcctgctcctgagagcccactgtcagacaagtggctggtccctgactgagcaggacccctacaacaacatcgtgcgcacagccatcgaagctatggccgccgtgtttggcggaacacagagcctgcacaccaacagctttgacgaggctctgggcctgcctaccgtgaagtctgccagaatcgcccggaacacccagatcatcatccaagaggaaagcggcatccccaaggtggcagatccttggggcggcagctacatgatggaatgcctgaccaacgacgtgtacgacgccgctctgaagctgatcaacgagatcgaagagatgggcggcatggctaaggctgtggccgagggaatccccaagctgagaatcgaggaatgcgccgccagacggcaggccagaattgatagcggaagcgaagtgatcgtgggcgtgaacaagtaccagctcgaaaaagaggacgccgtcgaggtcctggctatcgacaataccagcgtgcggaaccggcagattgagaagctgaagaagatcaagagcagccgcgatcaggccctggccgaaagatgtcttgctgccctgacagagtgtgccgccagcggcgacggaaatattctggctctggccgtggatgccagccgggctagatgtaccgtgggcgagattacagacgccctgaagaaggtgttcggcgagcacaaggccaacgacagaatggtgtctggcgcctacagacaagagtttggcgagagcaaagagatcaccagcgccatcaagcgggtccacaagttcatggaaagagaaggcaggcggcccagactgctggtggctaagatgggacaagacggccatgacagaggcgccaaagtgatcgccacaggctttgccgatctgggcttcgacgtggacatcggccctctgtttcagacccctagagaggtggcacagcaggccgttgatgccgatgttcacgctgtgggcatctctacactggctgccggacacaagacactggtgcccgaactgatcaaagagctgaacagcctgggcagacccgacatccttgtgatgtgtggcggagtgatcccaccgcaggactacgagttcctgtttgaagtgggcgtgtccaacgtgttcggccctggcacaagaatccctaaagccgccgtgcaggttctggacgacatcgagaagtgcctggaaaaaaagcagcagagcgtg

SEQ ID NO: 41 - DNA, 2247 bp, Artificial Sequence
  Synthetic Sequence
ctgcgggccaagaatcaactgttcctgctgtcccctcactacctgcggcaagtgaaagagagcagcggcagcagactgatccagcagagactgctgcatcagcagcagccactgcaccctgaatgggccgctctggctaagaagcagctcaagggcaagaaccccgaggacctgatctggcacacaccagagggcatcagcatcaagcccctgtactccaagcgggacacaatggatctgcccgaggaactgcctggcgtgaagccttttacaagaggcccctatcctaccatgtataccttcagaccctggaccatccggcagtacgccggcttttctaccgtggaagagagcaacaagttctacaaggacaacatcaaggccggccagcagggactgagcgtggcatttgatctggctacccacaggggctacgacagcgacaaccctagagtgcggggagatgttggaatggccggcgtggcaatcgacacagtggaagataccaagatcctgttcgacggcatccctctggaaaagatgagcgtgtccatgaccatgaacggcgctgtgatccccgtgctggctaactttattgtgaccggcgaggaacagggcgtgcccaaagaaaagctgaccggcaccatccagaacgacatcctgaaagagttcatggttcgaaacacctacatcttcccacctgagccgagcatgaagatcattgccgacatcttcgagtacaccgccaagcacatgcccaagttcaacagcatctccatcagcggctaccacatgcaagaggctggcgccgatgccatcctggaactggcttatacactggccgacggcctggaatactccagaacaggactgcaagccggcctgaccatcgatgagtttgcccctagactgagcttcttctggggcatcggcatgaacttctacatggaaatcgccaagatgagagccggcagacggctgtgggctcacctgatcgagaagatgttccagcctaagaacagcaagagcctgctcctgagagcccactgtcagacaagtggctggtccctgactgagcaggacccctacaacaacatcgtgcgcacagccatcgaagctatggccgccgtgtttggcggaacacagagcctgcacaccaacagctttgacgaggctctgggcctgcctaccgtgaagtctgccagaatcgcccggaacacccagatcatcatccaagaggaaagcggcatccccaaggtggcagatccttggggcggcagctacatgatggaatgcctgaccaacgacgtgtacgacgccgctctgaagctgatcaacgagatcgaagagatgggcggcatggctaaggctgtggccgagggaatccccaagctgagaatcgaggaatgcgccgccagacggcaggccagaattgatagcggaagcgaagtgatcgtgggcgtgaacaagtaccagctcgaaaaagaggacgccgtcgaggtcctggctatcgacaataccagcgtgcggaaccggcagattgagaagctgaagaagatcaagagcagccgcgatcaggccctggccgaaagatgtcttgctgccctgacagagtgtgccgccagcggcgacggaaatattctggctctggccgtggatgccagccgggctagatgtaccgtgggcgagattacagacgccctgaagaaggtgttcggcgagcacaaggccaacgacagaatggtgtctggcgcctacagacaagagtttggcgagagcaaagagatcaccagcgccatcaagcgggtccacaagttcatggaaagagaaggcaggcggcccagactgctggtggctaagatgggacaagacggccatgacagaggcgccaaagtgatcgccacaggctttgccgatctgggcttcgacgtggacatcggccctctgtttcagacccctagagaggtggcacagcaggccgttgatgccgatgttcacgctgtgggcatctctacactggctgccggacacaagacactggtgcccgaactgatcaaagagctgaacagcctgggcagacccgacatccttgtgatgtgtggcggagtgatcccaccgcaggactacgagttcctgtttgaagtgggcgtgtccaacgtgttcggccctggcacaagaatccctaaagccgccgtgcaggttctggacgacatcgagaagtgcctggaaaaaaagcagcagagcgtg

SEQ ID NO: 42 - DNA, 2256 bp, Artificial Sequence
  Synthetic Sequence
atgctgcgggccaagaatcaactgttcctgctgtcccctcactacctgcggcaagtgaaagagagcagcggcagcagactgatccagcagagactgctgcatcagcagcagccactgcaccctgaatgggccgctctggctaagaagcagctcaagggcaagaaccccgaggacctgatctggcacacaccagagggcatcagcatcaagcccctgtactccaagcgggacacaatggatctgcccgaggaactgcctggcgtgaagccttttacaagaggcccctatcctaccatgtataccttcagaccctggaccatccggcagtacgccggcttttctaccgtggaagagagcaacaagttctacaaggacaacatcaaggccggccagcagggactgagcgtggcatttgatctggctacccacaggggctacgacagcgacaaccctagagtgcggggagatgttggaatggccggcgtggcaatcgacacagtggaagataccaagatcctgttcgacggcatccctctggaaaagatgagcgtgtccatgaccatgaacggcgctgtgatccccgtgctggctaactttattgtgaccggcgaggaacagggcgtgcccaaagaaaagctgaccggcaccatccagaacgacatcctgaaagagttcatggttcgaaacacctacatcttcccacctgagccgagcatgaagatcattgccgacatcttcgagtacaccgccaagcacatgcccaagttcaacagcatctccatcagcggctaccacatgcaagaggctggcgccgatgccatcctggaactggcttatacactggccgacggcctggaatactccagaacaggactgcaagccggcctgaccatcgatgagtttgcccctagactgagcttcttctggggcatcggcatgaacttctacatggaaatcgccaagatgagagccggcagacggctgtgggctcacctgatcgagaagatgttccagcctaagaacagcaagagcctgctcctgagagcccactgtcagacaagtggctggtccctgactgagcaggacccctacaacaacatcgtgcgcacagccatcgaagctatggccgccgtgtttggcggaacacagagcctgcacaccaacagctttgacgaggctctgggcctgcctaccgtgaagtctgccagaatcgcccggaacacccagatcatcatccaagaggaaagcggcatccccaaggtggcagatccttggggcggcagctacatgatggaatgcctgaccaacgacgtgtacgacgccgctctgaagctgatcaacgagatcgaagagatgggcggcatggctaaggctgtggccgagggaatccccaagctgagaatcgaggaatgcgccgccagacggcaggccagaattgatagcggaagcgaagtgatcgtgggcgtgaacaagtaccagctcgaaaaagaggacgccgtcgaggtcctggctatcgacaataccagcgtgcggaaccggcagattgagaagctgaagaagatcaagagcagccgcgatcaggccctggccgaaagatgtcttgctgccctgacagagtgtgccgccagcggcgacggaaatattctggctctggccgtggatgccagccgggctagatgtaccgtgggcgagattacagacgccctgaagaaggtgttcggcgagcacaaggccaacgacagaatggtgtctggcgcctacagacaagagtttggcgagagcaaagagatcaccagcgccatcaagcgggtccacaagttcatggaaagagaaggcaggcggcccagactgctggtggctaagatgggacaagacggccatgacagaggcgccaaagtgatcgccacaggctttgccgatctgggcttcgacgtggacatcggccctctgtttcagacccctagagaggtggcacagcaggccgttgatgccgatgttcacgctgtgggcgtgtctacactggctgccggacacaagacactggtgcccgaactgatcaaagagctgaacagcctgggcagacccgacatccttgtgatgtgtggcggagtgatcccaccgcaggactacgagttcctgtttgaagtgggcgtgtccaacgtgttcggccctggcacaagaatccctaaagccgccgtgcaggttctggacgacatcgagaagtgcctggaaaaaaagcagcagagcgtgggatcc

SEQ ID NO: 43 - DNA, 2253 bp, Artificial Sequence
  Synthetic Sequence
ctgcgggccaagaatcaactgttcctgctgtcccctcactacctgcggcaagtgaaagagagcagcggcagcagactgatccagcagagactgctgcatcagcagcagccactgcaccctgaatgggccgctctggctaagaagcagctcaagggcaagaaccccgaggacctgatctggcacacaccagagggcatcagcatcaagcccctgtactccaagcgggacacaatggatctgcccgaggaactgcctggcgtgaagccttttacaagaggcccctatcctaccatgtataccttcagaccctggaccatccggcagtacgccggcttttctaccgtggaagagagcaacaagttctacaaggacaacatcaaggccggccagcagggactgagcgtggcatttgatctggctacccacaggggctacgacagcgacaaccctagagtgcggggagatgttggaatggccggcgtggcaatcgacacagtggaagataccaagatcctgttcgacggcatccctctggaaaagatgagcgtgtccatgaccatgaacggcgctgtgatccccgtgctggctaactttattgtgaccggcgaggaacagggcgtgcccaaagaaaagctgaccggcaccatccagaacgacatcctgaaagagttcatggttcgaaacacctacatcttcccacctgagccgagcatgaagatcattgccgacatcttcgagtacaccgccaagcacatgcccaagttcaacagcatctccatcagcggctaccacatgcaagaggctggcgccgatgccatcctggaactggcttatacactggccgacggcctggaatactccagaacaggactgcaagccggcctgaccatcgatgagtttgcccctagactgagcttcttctggggcatcggcatgaacttctacatggaaatcgccaagatgagagccggcagacggctgtgggctcacctgatcgagaagatgttccagcctaagaacagcaagagcctgctcctgagagcccactgtcagacaagtggctggtccctgactgagcaggacccctacaacaacatcgtgcgcacagccatcgaagctatggccgccgtgtttggcggaacacagagcctgcacaccaacagctttgacgaggctctgggcctgcctaccgtgaagtctgccagaatcgcccggaacacccagatcatcatccaagaggaaagcggcatccccaaggtggcagatccttggggcggcagctacatgatggaatgcctgaccaacgacgtgtacgacgccgctctgaagctgatcaacgagatcgaagagatgggcggcatggctaaggctgtggccgagggaatccccaagctgagaatcgaggaatgcgccgccagacggcaggccagaattgatagcggaagcgaagtgatcgtgggcgtgaacaagtaccagctcgaaaaagaggacgccgtcgaggtcctggctatcgacaataccagcgtgcggaaccggcagattgagaagctgaagaagatcaagagcagccgcgatcaggccctggccgaaagatgtcttgctgccctgacagagtgtgccgccagcggcgacggaaatattctggctctggccgtggatgccagccgggctagatgtaccgtgggcgagattacagacgccctgaagaaggtgttcggcgagcacaaggccaacgacagaatggtgtctggcgcctacagacaagagtttggcgagagcaaagagatcaccagcgccatcaagcgggtccacaagttcatggaaagagaaggcaggcggcccagactgctggtggctaagatgggacaagacggccatgacagaggcgccaaagtgatcgccacaggctttgccgatctgggcttcgacgtggacatcggccctctgtttcagacccctagagaggtggcacagcaggccgttgatgccgatgttcacgctgtgggcgtgtctacactggctgccggacacaagacactggtgcccgaactgatcaaagagctgaacagcctgggcagacccgacatccttgtgatgtgtggcggagtgatcccaccgcaggactacgagttcctgtttgaagtgggcgtgtccaacgtgttcggccctggcacaagaatccctaaagccgccgtgcaggttctggacgacatcgagaagtgcctggaaaaaaagcagcagagcgtgggatcc

SEQ ID NO: 44 - DNA, 2250 bp, Artificial Sequence; identical to SEQ ID NO: 40
  Synthetic Sequence

SEQ ID NO: 45 - DNA, 2247 bp, Artificial Sequence; identical to SEQ ID NO: 41
  Synthetic Sequence

SEQ ID NO: 46 - DNA, 2253 bp, Artificial Sequence
  Synthetic Sequence
atgctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaa

SEQ ID NO: 47 - DNA, 2250 bp, Artificial Sequence
  Synthetic Sequence
ctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaa

SEQ ID NO: 48 - DNA, 2253 bp, Artificial Sequence
  Synthetic Sequence
atgttaagagctaagaatcagctttttttactttcacctcattacctgaggcaggtaaaagaatcatcaggctccaggctcatacagcaacgacttctacaccagcaacagccccttcacccagaatgggctgccctggctaaaaagcagctgaaaggcaaaaacccagaagacctaatatggcacaccccggaagggatctctataaaacccttgtattccaagagagatactatggacttacctgaagaacttccaggagtgaagccattcacacgtggaccatatcctaccatgtatacctttaggccctggaccatccgccagtatgctggttttagtactgtggaagaaagcaataagttctataaggacaacattaaggctggtcagcagggattatcagttgcctttgatctggcgacacatcgtggctatgattcagacaaccctcgagttcgtggtgatgttggaatggctggagttgctattgacactgtggaagataccaaaattctttttgatggaattcctttagaaaaaatgtcagtttccatgactatgaatggagcagttattccagttcttgcaaattttatagtaactggagaagaacaaggtgtacctaaagagaagcttactggtaccatccaaaatgatatactaaaggaatttatggttcgaaatacatacatttttcctccagaaccatccatgaaaattattgctgacatatttgaatatacagcaaagcacatgccaaaatttaattcaatttcaattagtggataccatatgcaggaagcaggggctgatgccattctggagctggcctatactttagcagatggattggagtactctagaactggactccaggctggcctgacaattgatgaatttgcaccaaggttgtctttcttctggggaattggaatgaatttctatatggaaatagcaaagatgagagctggtagaagactctgggctcacttaatagagaaaatgtttcagcctaaaaactcaaaatctcttcttctaagagcacactgtcagacatctggatggtcacttactgagcaggatccctacaataatattgtccgtactgcaatagaagcaatggcagcagtatttggagggactcagtctttgcacacaaattcttttgatgaagctttgggtttgccaactgtgaaaagtgctcgaattgccaggaacacacaaatcatcattcaagaagaatctgggattcccaaagtggctgatccttggggaggttcttacatgatggaatgtctcacaaatgatgtttatgatgctgctttaaagctcattaatgaaattgaagaaatgggtggaatggccaaagctgtagctgagggaatacctaaacttcgaattgaagaatgtgctgcccgaagacaagctagaatagattctggttctgaagtaattgttggagtaaataagtaccagttggaaaaagaagacgctgtagaagttctggcaattgataatacttcagtgcgaaacaggcagattgaaaaacttaagaagatcaaatccagcagggatcaagctttggctgaacgttgtcttgctgcactaaccgaatgtgctgctagcggagatggaaatatcctggctcttgcagtggatgcatctcgggcaagatgtacagtgggagaaatcacagatgccctgaaaaaggtatttggtgaacataaagcgaatgatcgaatggtgagtggagcatatcgccaggaatttggagaaagtaaagagataacatctgctatcaagagggttcataaattcatggaacgtgaaggtcgcagacctcgtcttcttgtagcaaaaatgggacaagatggccatgacagaggagcaaaagttattgctacaggatttgctgatcttggttttgatgtggacataggccctcttttccagactcctcgtgaagtggcccagcaggctgtggatgcggatgtgcatgctgtgggcataagcaccctcgctgctggtcataaaaccctagttcctgaactcatcaaagaacttaactcccttggacggccagatattcttgtcatgtgtggaggggtgataccacctcaggattatgaatttctgtttgaagttggtgtttccaatgtatttggtcctgggactcgaattccaaaggctgccgttcaggtgcttgatgatattgagaagtgtttggaaaagaagcagcaatctgtataa

SEQ ID NO: 49 - DNA, 2250 bp, Artificial Sequence
  Synthetic Sequence
ttaagagctaagaatcagctttttttactttcacctcattacctgaggcaggtaaaagaatcatcaggctccaggctcatacagcaacgacttctacaccagcaacagccccttcacccagaatgggctgccctggctaaaaagcagctgaaaggcaaaaacccagaagacctaatatggcacaccccggaagggatctctataaaacccttgtattccaagagagatactatggacttacctgaagaacttccaggagtgaagccattcacacgtggaccatatcctaccatgtatacctttaggccctggaccatccgccagtatgctggttttagtactgtggaagaaagcaataagttctataaggacaacattaaggctggtcagcagggattatcagttgcctttgatctggcgacacatcgtggctatgattcagacaaccctcgagttcgtggtgatgttggaatggctggagttgctattgacactgtggaagataccaaaattctttttgatggaattcctttagaaaaaatgtcagtttccatgactatgaatggagcagttattccagttcttgcaaattttatagtaactggagaagaacaaggtgtacctaaagagaagcttactggtaccatccaaaatgatatactaaaggaatttatggttcgaaatacatacatttttcctccagaaccatccatgaaaattattgctgacatatttgaatatacagcaaagcacatgccaaaatttaattcaatttcaattagtggataccatatgcaggaagcaggggctgatgccattctggagctggcctatactttagcagatggattggagtactctagaactggactccaggctggcctgacaattgatgaatttgcaccaaggttgtctttcttctggggaattggaatgaatttctatatggaaatagcaaagatgagagctggtagaagactctgggctcacttaatagagaaaatgtttcagcctaaaaactcaaaatctcttcttctaagagcacactgtcagacatctggatggtcacttactgagcaggatccctacaataatattgtccgtactgcaatagaagcaatggcagcagtatttggagggactcagtctttgcacacaaattcttttgatgaagctttgggtttgccaactgtgaaaagtgctcgaattgccaggaacacacaaatcatcattcaagaagaatctgggattcccaaagtggctgatccttggggaggttcttacatgatggaatgtctcacaaatgatgtttatgatgctgctttaaagctcattaatgaaattgaagaaatgggtggaatggccaaagctgtagctgagggaatacctaaacttcgaattgaagaatgtgctgcccgaagacaagctagaatagattctggttctgaagtaattgttggagtaaataagtaccagttggaaaaagaagacgctgtagaagttctggcaattgataatacttcagtgcgaaacaggcagattgaaaaacttaagaagatcaaatccagcagggatcaagctttggctgaacgttgtcttgctgcactaaccgaatgtgctgctagcggagatggaaatatcctggctcttgcagtggatgcatctcgggcaagatgtacagtgggagaaatcacagatgccctgaaaaaggtatttggtgaacataaagcgaatgatcgaatggtgagtggagcatatcgccaggaatttggagaaagtaaagagataacatctgctatcaagagggttcataaattcatggaacgtgaaggtcgcagacctcgtcttcttgtagcaaaaatgggacaagatggccatgacagaggagcaaaagttattgctacaggatttgctgatcttggttttgatgtggacataggccctcttttccagactcctcgtgaagtggcccagcaggctgtggatgcggatgtgcatgctgtgggcataagcaccctcgctgctggtcataaaaccctagttcctgaactcatcaaagaacttaactcccttggacggccagatattcttgtcatgtgtggaggggtgataccacctcaggattatgaatttctgtttgaagttggtgtttccaatgtatttggtcctgggactcgaattccaaaggctgccgttcaggtgcttgatgatattgagaagtgtttggaaaagaagcagcaatctgtataa

SEQ ID NO: 50 - DNA, 2250 bp, Artificial Sequence
  Synthetic Sequence
atgctgcgggccaagaaccagctgttcctgctgagccctcactacctgcggcaggtgaaggagagcagcggcagccggctgatccagcagcggctgctgcaccagcagcagcccctgcaccccgagtgggccgccctggccaagaagcagctgaagggcaagaaccccgaggacctgatctggcacacgcccgagggcatcagcatcaagcccctgtacagcaagcgggacaccatggacctgcccgaggagctgcccggcgtgaagcccttcacccggggcccctaccccaccatgtacaccttccggccctggaccatccggcagtacgccggcttcagcaccgtggaggagagcaacaagttctacaaggacaacatcaaggccggccagcagggcctgagcgtggccttcgacctggccacccaccggggctacgacagcgacaacccacgggtgcggggcgacgtgggcatggccggcgtggccatcgacaccgtggaggacaccaagatcctgttcgacggcatccctctggagaagatgagcgtgagcatgaccatgaacggcgccgtgatccccgtgctggccaacttcatcgtgaccggcgaggagcagggcgtgcccaaggagaagctgaccggcaccatccagaacgacatcctgaaggagttcatggtgcggaacacctacatcttccctcccgagcccagcatgaagatcatcgccgacatcttcgagtacaccgccaagcacatgcccaagttcaacagcatcagcatcagcggctaccacatgcaggaggccggcgccgacgccatcctggagctggcctacaccctggccgacggcctggagtacagccggaccggcctgcaggccggcctgaccatcgacgagttcgcgccccggctgagcttcttctggggcatcggcatgaacttctacatggagatcgccaagatgcgggccggccggcggctgtgggcccacctgatcgagaagatgttccagcccaagaacagcaagagcctgctgctgcgggcccactgccagaccagcggctggagcctgaccgagcaggacccctacaacaacatcgtgcggaccgccatcgaggccatggccgccgtgttcggcggcacccagagcctgcacaccaacagcttcgacgaggccctgggcctgcccaccgtgaagagcgcccggatcgcccggaacacccagatcatcatccaggaggagagcggcatccccaaggtggccgacccctggggcggcagctacatgatggagtgcctgaccaacgacgtgtacgacgccgccctgaagctgatcaacgagatcgaggagatgggcggcatggccaaggccgtggccgagggcatccccaagctgcggatcgaggagtgcgccgcccggcggcaggcccggatcgacagcggcagcgaggtgatcgtgggcgtgaacaagtaccagctggagaaggaggacgccgtggaggtgctggccatcgacaacaccagcgtgcggaaccggcagatcgagaagctgaagaagatcaagagcagccgggaccaggccctggccgagcggtgcctggccgccctgaccgagtgcgccgccagcggcgacggcaacatcctggccctggccgtggacgccagccgggcccggtgcaccgtgggcgagatcaccgacgccctgaagaaggtgttcggcgagcacaaggccaacgaccggatggtgagcggcgcctaccggcaggagttcggcgagagcaaggagatcaccagcgccatcaagcgggtgcacaagttcatggagcgggagggccggcggccccggctgctggtggccaagatgggccaggacggccacgaccggggcgccaaggtgatcgccaccggcttcgccgacctgggcttcgacgtggacatcggcccactgttccagacgccccgggaggtggcccagcaggccgtggacgccgacgtgcacgccgtgggcgtgagcaccctggccgccggccacaagaccctggtgcccgagctgatcaaggagctgaacagcctgggccggcccgacatcctggtgatgtgcggcggcgtgatcccgccccaggactacgagttcctgttcgaggtgggcgtgagcaacgtgttcggccccggcacccggatccccaaggccgccgtgcaggtgctggacgacatcgagaagtgcctggagaagaagcagcagagcgtg

SEQ ID NO: 51 - DNA, 2247 bp, Artificial Sequence
  Synthetic Sequence
ctgcgggccaagaaccagctgttcctgctgagccctcactacctgcggcaggtgaaggagagcagcggcagccggctgatccagcagcggctgctgcaccagcagcagcccctgcaccccgagtgggccgccctggccaagaagcagctgaagggcaagaaccccgaggacctgatctggcacacgcccgagggcatcagcatcaagcccctgtacagcaagcgggacaccatggacctgcccgaggagctgcccggcgtgaagcccttcacccggggcccctaccccaccatgtacaccttccggccctggaccatccggcagtacgccggcttcagcaccgtggaggagagcaacaagttctacaaggacaacatcaaggccggccagcagggcctgagcgtggccttcgacctggccacccaccggggctacgacagcgacaacccacgggtgcggggcgacgtgggcatggccggcgtggccatcgacaccgtggaggacaccaagatcctgttcgacggcatccctctggagaagatgagcgtgagcatgaccatgaacggcgccgtgatccccgtgctggccaacttcatcgtgaccggcgaggagcagggcgtgcccaaggagaagctgaccggcaccatccagaacgacatcctgaaggagttcatggtgcggaacacctacatcttccctcccgagcccagcatgaagatcatcgccgacatcttcgagtacaccgccaagcacatgcccaagttcaacagcatcagcatcagcggctaccacatgcaggaggccggcgccgacgccatcctggagctggcctacaccctggccgacggcctggagtacagccggaccggcctgcaggccggcctgaccatcgacgagttcgcgccccggctgagcttcttctggggcatcggcatgaacttctacatggagatcgccaagatgcgggccggccggcggctgtgggcccacctgatcgagaagatgttccagcccaagaacagcaagagcctgctgctgcgggcccactgccagaccagcggctggagcctgaccgagcaggacccctacaacaacatcgtgcggaccgccatcgaggccatggccgccgtgttcggcggcacccagagcctgcacaccaacagcttcgacgaggccctgggcctgcccaccgtgaagagcgcccggatcgcccggaacacccagatcatcatccaggaggagagcggcatccccaaggtggccgacccctggggcggcagctacatgatggagtgcctgaccaacgacgtgtacgacgccgccctgaagctgatcaacgagatcgaggagatgggcggcatggccaaggccgtggccgagggcatccccaagctgcggatcgaggagtgcgccgcccggcggcaggcccggatcgacagcggcagcgaggtgatcgtgggcgtgaacaagtaccagctggagaaggaggacgccgtggaggtgctggccatcgacaacaccagcgtgcggaaccggcagatcgagaagctgaagaagatcaagagcagccgggaccaggccctggccgagcggtgcctggccgccctgaccgagtgcgccgccagcggcgacggcaacatcctggccctggccgtggacgccagccgggcccggtgcaccgtgggcgagatcaccgacgccctgaagaaggtgttcggcgagcacaaggccaacgaccggatggtgagcggcgcctaccggcaggagttcggcgagagcaaggagatcaccagcgccatcaagcgggtgcacaagttcatggagcgggagggccggcggccccggctgctggtggccaagatgggccaggacggccacgaccggggcgccaaggtgatcgccaccggcttcgccgacctgggcttcgacgtggacatcggcccactgttccagacgccccgggaggtggcccagcaggccgtggacgccgacgtgcacgccgtgggcgtgagcaccctggccgccggccacaagaccctggtgcccgagctgatcaaggagctgaacagcctgggccggcccgacatcctggtgatgtgcggcggcgtgatcccgccccaggactacgagttcctgttcgaggtgggcgtgagcaacgtgttcggccccggcacccggatccccaaggccgccgtgcaggtgctggacgacatcgagaagtgcctggagaagaagcagcagagcgtg

SEQ ID NO: 52 - protein, 750 aa, Artificial Sequence
  Synthetic Sequence
MLRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 53 - protein, 749 aa, Artificial Sequence
  Synthetic Sequence
LRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 54 - protein, 750 aa, Artificial Sequence [sequence omitted]

SEQ ID NO: 55 - protein, 749 aa, Artificial Sequence
  Synthetic Sequence
LRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGVSTLAAGHKTLVPELIKELNSLGRPDILVMCGGVIPPQDYEFLFEVGVSNVFGPGTRIPKAAVQVLDDIEKCLEKKQQSV

SEQ ID NO: 56 - DNA, 1062 bp, Artificial Sequence
  Synthetic Sequence
atgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttc

SEQ ID NO: 57 - DNA, 1062 bp, Artificial Sequence
  Synthetic Sequence
atgctgttcaacctgcgaatcctgctgaacaatgccgcttttcggaacgggcacaatttcatggtgaggaactttcgctgcggacagcccctccagaacaaggtccagctgaagggcagggacctgctgaccctgaaaaatttcacaggggaggaaatcaagtacatgctgtggctgtcagccgatctgaagttccggatcaagcagaagggcgaatatctgcctctgctccagggcaaaagcctggggatgatcttcgaaaagcgcagtactcggaccagactgtcaacagagactggattcgcactgctgggaggacacccatgttttctgaccacacaggacattcatctgggagtgaacgagtccctgaccgacacagcacgcgtcctgagctccatggctgatgcagtgctggctcgagtctacaaacagtctgacctggataccctggccaaggaagcttctatcccaatcattaatggcctgagtgacctgtatcaccccatccagattctggccgattacctgaccctccaggagcattattctagtctgaaagggctgacactgagctggattggggacggaaacaatatcctgcactccattatgatgagcgccgccaagtttggaatgcacctccaggctgcaaccccaaaaggctacgaacccgatgcctccgtgacaaagctggcagaacagtatgccaaagagaacggcactaagctgctgctcaccaatgaccctctggaggccgctcacggaggcaacgtgctgatcactgatacctggattagtatgggacaggaggaagagaagaagaagcggctccaggccttccagggctaccaggtgacaatgaaaactgctaaggtcgcagccagcgactggacctttctgcattgcctgcccagaaagcctgaagaggtggacgatgaggtcttctactcacccagaagcctggtgtttcctgaagctgagaataggaagtggacaatcatggcagtgatggtcagcctgctgactgattattcccctcagctccagaaaccaaagttc

SEQ ID NO: 58 - DNA, 1059 bp, Artificial Sequence
  Synthetic Sequence
ctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttc

SEQ ID NO: 59 - DNA, 1059 bp, Artificial Sequence
  Synthetic Sequence
ctgttcaacctgcgaatcctgctgaacaatgccgcttttcggaacgggcacaatttcatggtgaggaactttcgctgcggacagcccctccagaacaaggtccagctgaagggcagggacctgctgaccctgaaaaatttcacaggggaggaaatcaagtacatgctgtggctgtcagccgatctgaagttccggatcaagcagaagggcgaatatctgcctctgctccagggcaaaagcctggggatgatcttcgaaaagcgcagtactcggaccagactgtcaacagagactggattcgcactgctgggaggacacccatgttttctgaccacacaggacattcatctgggagtgaacgagtccctgaccgacacagcacgcgtcctgagctccatggctgatgcagtgctggctcgagtctacaaacagtctgacctggataccctggccaaggaagcttctatcccaatcattaatggcctgagtgacctgtatcaccccatccagattctggccgattacctgaccctccaggagcattattctagtctgaaagggctgacactgagctggattggggacggaaacaatatcctgcactccattatgatgagcgccgccaagtttggaatgcacctccaggctgcaaccccaaaaggctacgaacccgatgcctccgtgacaaagctggcagaacagtatgccaaagagaacggcactaagctgctgctcaccaatgaccctctggaggccgctcacggaggcaacgtgctgatcactgatacctggattagtatgggacaggaggaagagaagaagaagcggctccaggccttccagggctaccaggtgacaatgaaaactgctaaggtcgcagccagcgactggacctttctgcattgcctgcccagaaagcctgaagaggtggacgatgaggtcttctactcacccagaagcctggtgtttcctgaagctgagaataggaagtggacaatcatggcagtgatggtcagcctgctgactgattattcccctcagctccagaaaccaaagttc

SEQ ID NO: 60 - protein, 354 aa, Artificial Sequence
  Synthetic Sequence
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 61 - protein, 353 aa, Artificial Sequence
  Synthetic Sequence
LFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 62 - protein, 354 aa, Artificial Sequence
  Synthetic Sequence
MLSNLRILLNNAALRKGHTSVVRHFWCGKPVQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 63 - protein, 353 aa, Artificial Sequence
  Synthetic Sequence
LSNLRILLNNAALRKGHTSVVRHFWCGKPVQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 64 - protein, 461 aa, Artificial Sequence
  Synthetic Sequence
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 65 - DNA, 1383 bp, Artificial Sequence
  Synthetic Sequence
atgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcacc

(19) United States
(12) Patent Application Publication
(10) Pub. No.: US 2024/0189446 A1
(43) Pub. Date: Jun. 13, 2024

(54) COMPOSITIONS AND METHODS FOR DELIVERY OF NUCLEIC ACIDS

(71) Applicant: Poseida Therapeutics, Inc., San Diego, CA (US)

(72) Inventors: Joshua RYCHAK, San Diego, CA (US); Sean ESSEX, San Diego, CA (US); Matthew BUTCHER, San Diego, CA (US); Peter GRIFFIN, San Diego, CA (US); Blair MADISON, San Diego, CA (US); Renata MARTIN, San Diego, CA (US); Neha PARAYATH, San Diego, CA (US); Michael J. BENNETT, San Diego, CA (US); Khalid A. HAJJ, San Diego, CA (US); Oscar ALVAREZ, San Diego, CA (US); Dan J. SMITH, San Diego, CA (US)

(21) Appl. No.: 18/263,793

(22) PCT Filed: Feb. 23, 2022
(86) PCT No.: PCT/US2022/017570

§ 371 Date: Aug. 1, 2023

Related U.S. Application Data
(60) Provisional application No. 63/152,517, filed on Feb. 23, 2021.
(60) Provisional application No. 63/156,649, filed on Mar. 4, 2021.
(60) Provisional application No. 63/164,174, filed on Mar. 22, 2021.
(60) Provisional application No. 63/197,946, filed on Jun. 7, 2021.

(51) Int. Cl. A61K 48/00; A61K 9/51; A61K 31/7088; A61K 38/18; A61K 38/37; A61K 38/45; A61K 38/46; A61K 38/48; A61P 7/04

(52) U.S. Cl. CPC A61K 48/0041; A61K 9/5123; A61K 9/5146; A61K 31/7088; A61K 38/1816; A61K 38/37; A61K 38/45; A61K 38/465; A61K 38/4846; A61P 7/04; C12Y 201/03003; C12Y 304/21022

(57) ABSTRACT

The present disclosure provides compositions and methods for the genetic modification of cells, including, but not limited to, hepatocytes. The compositions and methods can comprise lipid nanoparticles, wherein the lipid nanoparticles comprise at least one bioreducible ionizable cationic lipid, at least one structural lipid, at least one phospholipid and at least one PEGylated lipid.

### RELATED APPLICATIONS

This application claims priority to, and the benefit of, U.S. Provisional Application No. 63/152,517, filed on Feb. 23, 2021, U.S. Provisional Application No. 63/156,649, filed on Mar. 4, 2021, U.S. Provisional Application No. 63/164,174, filed on Mar. 22, 2021, and U.S. Provisional Application No. 63/197,946, filed on Jun. 7, 2021. The contents of each of the aforementioned patent applications are incorporated herein by reference in their entireties.

### SEQUENCE LISTING

The instant application contains a Sequence Listing which has been submitted in ASCII format via EFS-Web and is hereby incorporated by reference in its entirety. Said ASCII copy, created on Feb. 23, 2022, is named “POTH-066_001WO_SeqList.txt” and is about 320,338 bytes in size.

### FIELD

The present disclosure relates generally to novel lipid nanoparticles (“LNPs”) comprising bioreducible ionizable cationic lipids, methods of preparing these LNPs, and the use of these LNPs for gene therapy and cell-based therapy applications.

### BACKGROUND

There has been a long-felt but unmet need in the art for compositions and methods for delivering nucleic acids to cells and for genetically modifying cells in vivo, ex vivo and in vitro. Widely accepted gene delivery and genetic modification techniques, such as the use of viral vectors, including AAVs, can cause acute toxicity and harmful side-effects in patients. The present disclosure provides improved compositions, methods and kits for the delivery of nucleic acids to various types of cells, including hepatocytes, in vivo, ex vivo and in vitro. More specifically, the present disclosure provides improved lipid nanoparticle compositions and methods of using the same. These lipid nanoparticle compositions and methods allow for the delivery of specific types of nucleic acids (e.g. RNA) to liver cells with high efficiency and low toxicity. Moreover, the lipid nanoparticle compositions of the present disclosure exhibit improved storage stability, which is advantageous in clinical and commercial settings. Thus, the compositions and methods of the present disclosure have wide applicability to a diverse number of fields, including gene therapy and the production of cell-based therapeutics.

### SUMMARY

In some aspects, provided are novel lipid nanoparticles (“LNPs”) comprising a bioreducible ionizable cationic lipid. In one aspect, the bioreducible ionizable cationic lipid is Coatsome SS-OP.

In one aspect, provided are pharmaceutical compositions, comprising a composition of the present disclosure and at least one pharmaceutically-acceptable excipient or diluent.

In one aspect, provided are methods of delivering at least one nucleic acid to at least one cell comprising contacting the at least one cell with at least one composition of the present disclosure.

In one aspect, provided are methods of genetically modifying at least one cell comprising contacting the at least one cell with at least one composition of the present disclosure.

In one aspect, provided are methods of treating at least one disease or disorder in a subject in need thereof comprising administering to the subject at least one therapeutically effective amount of at least one composition of the present disclosure.

In one aspect, provided are methods of delivering at least one nucleic acid to at least one cell comprising contacting the at least one cell with at least one composition of the present disclosure.

In one aspect, provided are cells modified according to methods of the present disclosure.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a series of graphs showing expression of human FVIII in adult mice administered LNPs of the present disclosure.

FIG. 2 is a series of graphs showing expression of human FIX in mice administered LNPs of the present disclosure.

FIG. 3 is a series of graphs showing the mean diameter and polydispersity index (PDI) of various LNP compositions of the present disclosure over the course of a 7-day incubation at 0.1 mg/mL and at a temperature of 4° C.

FIG. 4 is a graph showing expression of human FVIII in mice administered LNPs of the present disclosure.

FIG. 5 is a graph showing expression of human erythropoietin (hEPO) in Non-Human Primates (NHPs) administered LNPs of the present disclosure.

FIG. 6 is a series of graphs showing the levels of liver enzymes in NHPs administered LNPs of the present disclosure.

FIG. 7 is a graph showing survival of mice administered Human OTC (hOTC) transposon AAV viral vector particles and Human OTC (hOTC) transposon AAV viral vector particles in combination with SPB LNPs of the present disclosure.

FIG. 8 is a series of graphs showing integrated viral copy number (VCN) and hOTC mRNA levels in mice administered Human OTC (hOTC) transposon AAV viral vector particles in combination with SPB LNPs of the present disclosure.

FIG. 9 is a series of graphs showing the survival of and the orotic acid levels in mice administered Human OTC (hOTC) transposon AAV viral vector particles in combination with SPB LNPs of the present disclosure.

### DETAILED DESCRIPTION

The present disclosure provides novel lipid nanoparticle (LNP) compositions comprising bioreducible ionizable cationic lipids, methods for preparing said compositions, and methods of using said compositions. In a non-limiting example, the compositions and methods of the present disclosure can be used for gene delivery in the context of gene therapies. In another non-limiting example, the compositions and methods of the present disclosure can be used in the context of cell-based therapeutics. In another non-limiting example, the compositions and methods of the present disclosure can be broadly used to deliver a nucleic acid to induce the expression of a therapeutic protein, including, but not limited to, secreted therapeutic proteins. In a non-limiting example, the compositions and methods of the present disclosure can be broadly used to deliver a nucleic acid to liver cells, in vivo, ex vivo or in vitro, for the treatment of certain liver disorders.

The bioreducible ionizable cationic lipids of the present disclosure are biodegradable, thereby allowing the bioreducible ionizable cationic lipids to be broken down and metabolized in an animal. Without wishing to be bound by theory, this bioreducibility advantageously lessens cationic lipid-associated cytotoxicity.

In some aspects of the compositions and methods of the present disclosure, a bioreducible ionizable cationic lipid for use in the LNP compositions can be ssPalmO-Ph-P4C2. As would be appreciated by the skilled artisan, ssPalmO-Ph-P4C2 has the following structure:

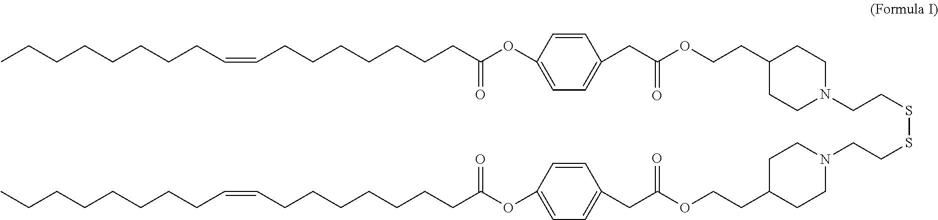 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

As would be appreciated by the skilled artisan, ssPalmO-Ph-P4C2 can also be referred to as Coatsome® SS-OP, ssPalmO-Phe-P4C2, ssPalmO-Phenyl-P4C2, ssPalmO-Phe and ssPalmO-Ph. Accordingly, ssPalmO-Ph-P4C2, Coatsome® SS-OP, ssPalmO-Phe-P4C2, ssPalmO-Phenyl-P4C2, ssPalmO-Phe and ssPalmO-Ph are used interchangeably herein to refer to the bioreducible ionizable cationic lipid with the chemical structure put forth in Formula I.

Without wishing to be bound by theory, three specific segments of ssPalmO-Ph-P4C2 facilitate its biodegradation. First, the tertiary amine of each piperidine ring is an acidic pH-responsive cation-charging unit. Upon endocytosis, the tertiary amine moieties become positively charged in response to the acidic, intracellular endosomal compartment. These are now able to interact and destabilize the membrane and this leads to endosomal escape. Once in the cytosol, the disulfide bond is susceptible to reduction by glutathione generating two free sulfhydryl groups. The resulting high concentration of free thiols further leads to nucleophilic reaction and the particle undergoes self-degradation/collapse via thioesterification and releases the payload in the cytosol. This is defined as HyPER or Hydrolysis accelerated by the intra-Particle Enrichment of Reactant and potentially eliminates the potentially toxic side effects of cationic lipids in general.

Compositions of the Present Disclosure—Lipid Nanoparticles

The present disclosure provides a composition comprising at least one lipid nanoparticle comprising at least one cationic lipid and at least one nucleic acid molecule. In some aspects, a lipid nanoparticle can further comprise at least one structural lipid. In some aspects, a lipid nanoparticle can further comprise at least one phospholipid. In some aspects, a lipid nanoparticle can further comprise at least one PEGylated lipid.

Accordingly, the present disclosure provides compositions comprising at least one lipid nanoparticle, wherein the at least one lipid nanoparticle comprises at least one cationic lipid, at least one nucleic acid molecule, at least one structural lipid, at least one phospholipid and at least one PEGylated lipid.

Bioreducible Ionizable Cationic Lipids

In some aspects, a cationic lipid can be a bioreducible ionizable cationic lipid. Accordingly, the present disclosure provides compositions comprising at least one lipid nanoparticle, wherein the at least one lipid nanoparticle comprises at least one bioreducible ionizable cationic lipid.

As used herein, the term “bioreducible ionizable cationic lipid” is used in its broadest sense to refer to a cationic lipid comprising: at least one tertiary amine, at least one disulfide group, at least one group comprising a bond that is susceptible to cleavage by thioesterification, and further comprising at least two saturated or unsaturated hydrocarbon chains. Exemplary bioreducible ionizable cationic lipids include, but are not limited to, those described in Akita et al., (2020) Biol. Phar. Bull. 43:1617-1625, the contents of which is incorporated herein by reference in their entirety.

In some aspects, a bioreducible ionizable cationic lipid can comprise at least two tertiary amines. In some aspects, at least one tertiary amine cane be a substituted piperidinyl group. In some aspects, each tertiary amine can be a substituted piperidinyl group. In some aspects, a bioreducible ionizable cationic lipid can comprise at least one disulfide bond. In some aspects, the sulfur atoms of the disulfide bond are linked to the nitrogen of the piperdinyl ring via an alkylene group, thereby forming two tertiary amine groups flanking the disulfide bond. In some aspects, at least one of the alkylene groups is an ethylene group. In some aspects, each of the alkylene groups is an ethylene group.

In some aspects, an at least one group comprising a bond that is susceptible to cleavage by thioesterification can be a phenyl ester group. In some aspects, a bioreducible ionizable cationic lipid can comprise at least two phenyl ester groups. In some aspects, at least one of the at least two saturated or unsaturated hydrocarbon chains is an unsaturated hydrocarbon chain. In some aspects, each of the least two saturated or unsaturated hydrocarbon chains is an unsaturated hydrocarbon chain. In some aspects, an unsaturated hydrocarbon chain can be an octadecene. In some aspects, an octadecene can be (Z)-octadec-9-ene. In some aspects, an (Z)-octadec-9-ene group can linked to a phenyl ester group of the bioreducible ionizable cationic lipid.

Exemplary bioreducible ionizable cationic lipids and methods of preparing such lipids useful in the methods of the present disclosure include those disclosed in International Patent Application No. PCT/JP2016/052690, published as WO/2016/121942 and International Patent Application No. PCT/JP2019/012302, published as WO/2019/188867, the contents of each of which are incorporated herein by reference in their entirety. Accordingly, the present disclosure provides compositions comprising at least one lipid nanoparticle, wherein the at least one lipid nanoparticle comprises any one of the bioreducible ionizable cationic lipids put forth in WO/2016/121942 and WO/2019/188867.

Accordingly, the present disclosure provides compositions comprising at least one lipid nanoparticle, wherein the at least one lipid nanoparticle comprises at least one bioreducible ionizable cationic lipid, at least one nucleic acid molecule, at least one structural lipid, at least one phospholipid and at least one PEGylated lipid.

In some aspects, the bioreducible ionizable cationic lipid can be ssPalmO-Ph-P4C2, having the structure put forth in Formula I (see Akita et al., (2020) Biol. Phar. Bull. 43:1617-1625, the contents of which are incorporated by reference in their entirety).

As described herein, the LNP compositions of the present disclosure that comprise at least one bioreducible ionizable cationic lipid advantageously exhibit significantly reduced toxicity in animals as compared to LNP compositions comprising non-bioreducible ionizable cationic lipids. In particular, administration the LNP compositions of the present disclosure surprisingly does not result in any body weight loss. In fact, the LNP compositions of the present disclosure are so non-toxic that animals administered the LNPs actually gain body weight, even when administered amounts of LNPs that exceed the lethal dose of LNP compositions comprising non-bioreducible ionizable cationic lipids.

LNP Components

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one bioreducible ionizable cationic lipid by moles.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one bioreducible ionizable cationic lipid by moles.

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one structural lipid by moles.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one structural lipid by moles.

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5%, or about 60%, or about 62.5%, or about 65%, or about 67.5%, or about 70% of at least one phospholipid by moles.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5%, or at least about 60%, or at least about 62.5%, or at least about 65%, or at least about 67.5%, or at least about 70% of at least one phospholipid by moles.

In some aspects, an LNP of the present disclosure can comprise about 0.25%, or about 0.5%, or about 0.75%, or about 1.0%, or about 1.25%, or about 1.5%, or about 1.75%, or about 2.0%, or at least about or about 2.5%, or about 5% of at least one PEGylated lipid by moles.

In some aspects, an LNP of the present disclosure can comprise at least about 0.25%, or at least about 0.5%, or at least about 0.75%, or at least about 1.0%, or at least about 1.25%, or at least about 1.5%, or at least about 1.75%, or at least about 2.0%, or at least about or at least about 2.5%, or at least about 5% of at least one PEGylated lipid by moles.

Structural Lipids

In some aspects, a structural lipid can be a steroid. In some aspects, a structural lipid can be a sterol. In some aspects, a structural lipid can comprise cholesterol. In some aspects, a structural lipid can comprise ergosterol. In some aspects, a structural lipid can be a phytosterol.

Phospholipid

As used herein, the term “phospholipid” is used in its broadest sent to refer to any amphiphilic molecule that comprises a polar (hydrophilic) headgroup comprising phosphate and two hydrophobic fatty acid chains.

In some aspects of the lipid nanoparticles of the present disclosure, a phospholipid can comprise dioleoylphosphatidylethanolamine (DOPE).

In some aspects of the lipid nanoparticles of the present disclosure, a phospholipid can comprise DOPC (1,2-Dioleoyl-sn-glycero-3-phosphocholine).

In some aspects of the lipid nanoparticles of the present disclosure, a phospholipid can comprise DSPC (1,2-Distearoyl-sn-glycero-3-phosphocholine).

In some aspects, a phospholipid can comprise DDPC (1,2-Didecanoyl-sn-glycero-3-phosphocholine), DEPA-NA (1,2-Dierucoyl-sn-glycero-3-phosphate (Sodium Salt)), DEPC (1,2-Dierucoyl-sn-glycero-3-phosphocholine), DEPE (1,2-Dierucoyl-sn-glycero-3-phosphoethanolamine), DEPG-NA (1,2-Dierucoyl-sn-glycero-3[Phospho-rac-(1-glycerol) (Sodium Salt)), DLOPC (1,2-Dilinoleoyl-sn-glycero-3-phosphocholine), DLPA-NA (1,2-Dilauroyl-sn-glycero-3-phosphate (Sodium Salt)), DLPC (1,2-Dilauroyl-sn-glycero-3-phosphocholine), DLPE (1,2-Dilauroyl-sn-glycero-3-phosphoethanolamine), DLPG-NA (1,2-Dilauroyl-sn-glycero-3[Phospho-rac-(1-glycerol) (Sodium Salt)), DLPG-NH4 (1,2-Dilauroyl-sn-glycero-3[Phospho-rac-(1-glycerol) (Ammonium Salt)), DLPS-NA (1,2-Dilauroyl-sn-glycero-3-phosphoserine (Sodium Salt)), DMPA-NA (1,2-Dimyristoyl-sn-glycero-3-phosphate (Sodium Salt)), DMPC (1,2-Dimyristoyl-sn-glycero-3-phosphocholine), DMPE (1,2-Dimyristoyl-sn-glycero-3-phosphoethanolamine), DMPG-NA (1,2-Dimyristoyl-sn-glycero-3[Phospho-rac-(1-glycerol) (Sodium Salt)), DMPG-NH4 (1,2-Dimyristoyl-sn-glycero-3[Phospho-rac-(1-glycerol) (Ammonium Salt)), DMPG-NH4/NA (1,2-Dimyristoyl-sn-glycero-3[Phospho-rac-(1-glycerol) (Sodium/Ammonium Salt)), DMPS-NA (1,2-Dimyristoyl-sn-glycero-3-phosphoserine (Sodium Salt)), DOPA-NA (1,2-Dioleoyl-sn-glycero-3-phosphate (Sodium Salt)), DOPC (1,2-Dioleoyl-sn-glycero-3-phosphocholine), DOPE (1,2-Dioleoyl-sn-glycero-3-phosphoethanolamine), DOPG-NA (1,2-Dioleoyl-sn-glycero-3[Phospho-rac-(1-glycerol) (Sodium Salt)), DOPS-NA (1,2-Dioleoyl-sn-glycero-3-phosphoserine (Sodium Salt)), DPPA-NA (1,2-Dipalmitoyl-sn-glycero-3-phosphate (Sodium Salt)), DPPC (1,2-Dipalmitoyl-sn-glycero-3-phosphocholine), DPPE (1,2-Dipalmitoyl-sn-glycero-3-phosphoethanolamine), DPPG-NA (1,2-Dipalmitoyl-sn-glycero-3[Phospho-rac-(1-glycerol) (Sodium Salt)), DPPG-NH4 (1,2-Dipalmitoyl-sn-glycero-3[Phospho-rac-(1-glycerol) (Ammonium Salt)), DPPS-NA (1,2-Dipalmitoyl-sn-glycero-3-phosphoserine (Sodium Salt)), DSPA-NA (1,2-Distearoyl-sn-glycero-3-phosphate (Sodium Salt)), DSPC (1,2-Distearoyl-sn-glycero-3-phosphocholine), DSPE (1,2-Distearoyl-sn-glycero-3-phosphoethanolamine), DSPG-NA (1,2-Distearoyl-sn-glycero-3[Phospho-rac-(1-glycerol) (Sodium Salt)), DSPG-NH4 (1,2-Distearoyl-sn-glycero-3[Phospho-rac-(1-glycerol) (Ammonium Salt)), DSPS-NA (1,2-Distearoyl-sn-glycero-3-phosphoserine (Sodium Salt)), EPC (Egg-PC), HEPC(Hydrogenated Egg PC), HSPC (Hydrogenated Soy PC), LYSOPC MYRISTIC (1-Myristoyl-sn-glycero-3-phosphocholine), LYSOPC PALMITIC (1-Palmitoyl-sn-glycero-3-phosphocholine), LYSOPC STEARIC (1-Stearoyl-sn-glycero-3-phosphocholine), Milk Sphingomyelin (MPPC; 1-Myristoyl-2-palmitoyl-sn-glycero 3-phosphocholine), MSPC (1-Myristoyl-2-stearoyl-sn-glycero-3-phosphocholine), PMPC (1-Palmitoyl-2-myristoyl-sn-glycero-3-phosphocholine), POPC (1-Palmitoyl-2-oleoyl-sn-glycero-3-phosphocholine), POPE (1-Palmitoyl-2-oleoyl-sn-glycero-3-phosphoethanolamine), POPG-NA (1-Palmitoyl-2-oleoyl-sn-glycero-3[Phospho-rac-(1-glycerol)] (Sodium Salt)), PSPC (1-Palmitoyl-2-stearoyl-sn-glycero-3-phosphocholine), SMPC (1-Stearoyl-2-myristoyl-sn-glycero-3-phosphocholine), SOPC (1-Stearoyl-2-oleoyl-sn-glycero-3-phosphocholine), SPPC (1-Stearoyl-2-palmitoyl-sn-glycero-3-phosphocholine), or any combination thereof.

PEGylated Lipid

As used herein, the term “PEGylated lipid” is used to refer to any lipid that is modified (e.g. covalently linked to) at least one polyethylene glycol molecule. In some aspects, a PEGylated lipid can comprise 1,2-dimyristoyl-rac-glycero-3-methoxypolyethylene glycol-2000, hereafter referred to as DMG-PEG2000.

Nucleic Acids

In some aspects, a lipid nanoparticle can comprise at least one nucleic acid molecule. In some aspects, a lipid nanoparticle can comprise a plurality of nucleic acid molecules. In some aspects, the at least one nucleic acid molecule or the plurality of nucleic acid molecules can be formulated in a lipid nanoparticle.

In some aspects, a nucleic acid molecule can be a synthetic nucleic acid molecule. In some aspects, a nucleic acid molecule can be a non-naturally occurring nucleic acid molecule. In some aspects, a non-naturally occurring nucleic acid molecule can comprise at least one non-naturally occurring nucleotide. The at least one non-naturally occurring nucleotide can be any non-naturally occurring nucleotide known in the art. In some aspects, a nucleic acid molecule can be a modified nucleic acid molecule. In some aspects, a modified nucleic acid molecule can comprise at least one modified nucleotide. The at least one modified nucleotide can be any modified nucleic acid known in the art.

In some aspects, a lipid nanoparticle can comprise lipid and nucleic acid at a specified ratio (weight/weight).

In some aspects, a lipid nanoparticle comprising at least one nucleic acid molecule can comprise lipid and nucleic acid at a ratio of about 5:1 to about 15:1, or about 10:1 to about 20:1, or about 15:1 to about 25:1, or about 20:1 to about 30:1, or about 25:1 to about 35:1 or about 30:1 to about 40:1, or about 35:1 to about 45:1, or about 40:1 to about 50:1, or about 45:1 to about 55:1, or about 50:1 to about 60:1, or about 55:1 to about 65:1, or about 60:1 to about 70:1, or about 65:1 to about 75:1, or about 70:1 to about 80:1, or about 75:1 to about 85:1, or about 80:1 to about 90:1, or about 85:1 to about 95:1, or about 90:1 to about 100:1, or about 95:1 to about 105:1, or about 100:1 to about 110:1, or about 105:1 to about 115:1, or about 110:1 to about 120:1, or about 115:1 to about 125:1, or about 120:1 to about 130:1, or about 125:1 to about 135:1, or about 130:1 to about 140:1, or about 135:1 to about 145:1, or about 140:1 to about 150:1, lipid:nucleic acid, weight/weight.

In some aspects, a lipid nanoparticle can comprise lipid and nucleic acid at a ratio of about 5:1, or about 10:1, or about 15:1, or about 20:1, or about 25:1, or about 30:1, or about 35:1, or about 40:1, or about 45:1, or about 50:1, or about 55:1, or about 60:1, or about 65:1, or about 70:1, or about 75:1, or about 80:1, or about 85:1, or about 90:1, or about 95:1, or about 100:1, or about 105:1, or about 110:1, or about 115:1, or about 120:1, or about 125:1, or about 130:1, or about 135:1, or about 140:1, or about 145:1, or about 150:1, or about 200:1, lipid:nucleic acid, weight/weight.

In some aspects, a lipid nanoparticle can comprise lipid and nucleic acid at a ratio of about 10:1, or about 17.5:1, or about 25:1, lipid:nucleic acid, weight/weight.

In some aspects, a nucleic acid molecule can be an RNA molecule. Thus, in some aspects, a lipid nanoparticle can comprise at least one RNA molecule. In some aspects, an RNA molecule can be an mRNA molecule. In some aspects, an mRNA molecule can comprise a 5′-CAP.

In some aspects, an mRNA molecule can be capped using any method and/or capping moiety known in the art. An mRNA molecule can be capped with m7G(5′)ppp(5′)G moiety. A m7G(5′)ppp(5′)G moiety is also referred to herein as a “Cap0”. An mRNA molecule can be capped with a CleanCap® moiety. A CleanCap® moiety can comprise a m7G(5′)ppp(5′)(2′OMeA) (CleanCap® AG) moiety. A CleanCap® moiety can comprise a m7G(5′)ppp(5′)(2′OMeG) (CleanCap® GG) moiety. An mRNA molecule can be capped with an anti-reverse cap analog (ARCA®) moiety. An ARCA® moiety can comprise a m7(3′-O-methyl)G(5′)ppp(5′)G moiety. An mRNA molecule can be capped with a CleanCap® 3′OMe moiety (CleanCap®+ARCA®).

In some aspects, an mRNA molecule can comprise at least one modified nucleic acid.

Modified nucleic acids can include, but are not limited to, 5-methoxyuridine (5moU), Ni-methylpseudouridine (me¹Ψ), pseudouridine (Ψ), 5-methylcytidine (5-MeC).

In some aspects, a nucleic acid molecule can be a DNA molecule. Thus, in some aspects, a lipid nanoparticle can comprise at least one DNA molecule. In some aspects, a DNA molecule can be a circular DNA molecule, such as, but not limited to, a DNA plasmid. In some aspects, a lipid nanoparticle can comprise a DNA plasmid. In some aspects, a DNA molecule can be a linearized DNA molecule, such as, but not limited to, a linearized DNA plasmid. In some aspect, a DNA molecule can be a DoggyBone DNA molecule. In some aspects, a DNA molecule can be a DNA nanoplasmid.

A DNA plasmid can comprise can be at least about 0.25 kb, or at least about 0.5 kb, or at least about 0.75 kb, or at least about 1.0 kb, or at least about 1.25 kb, or at least about 1.5 kb, or at least about 1.75 kb, or at least about 2.0 kb, or at least about 2.25 kb, or at least about 2.5 kb, or at least about 2.75 kb, or at least about 3.0 kb, or at least about 3.25 kb, or at least about 3.5 kb, or at least about 3.75 kb, or at least about 4.0 kb, or at least about 4.25 kb, or at least about 4.5 kb, or at least about 4.75 kb, or at least about 5.0 kb, or at least about 5.25 kb, or at least about 5.5 kb, or at least about 5.75 kb, or at least about 6.0 kb, or at least about 6.25 kb, or at least about 6.5 kb, or at least about 6.75 kb, or at least about 7.0 kb, or at least about 7.25 kb, or at least about 7.5 kb, or at least about 7.75 kb, or at least about 8.0 kb, or at least about 8.25 kb, or at least about 8.5 kb, or at least about 8.75 kb, or at least about 9.0 kb, or at least about 9.25 kb, or at least about 9.5 kb, or at least about 9.75 kb, or at least about 10.0 kb, or at least about 10.25 kb, or at least about 10.5 kb, or at least about 10.75 kb, or at least about 11.0 kb, or at least about 11.25 kb, or at least about 11.5 kb, or at least about 11.75 kb, or at least about 12 kb, or at least about 12.25 kb, or at least about 12.5 kb, or at least about 12.75 kb, or at least about 13.0 kb, or at least about 13.25 kb, or at least about 13.5 kb, or at least about 13.75 kb, or at least about 14.0 kb, or at least about 14.25 kb, or at least about 14.5 kb, or at least about 14.75 kb or at least about 15.0 kb in length.

LNP Compositions

In some aspects, a lipid nanoparticle can comprise at least one nucleic acid molecule, at least one bioreducible ionizable cationic lipid, and at least one structural lipid. In some aspects, a lipid nanoparticle can comprise at least one nucleic acid molecule, at least one bioreducible ionizable cationic lipid, and at least one PEGylated lipid. In some aspects, an at least one bioreducible ionizable cationic lipid can be ssPalmO-Ph-P4C2.

In some aspects, an at least one structural lipid can be a mixture of two structural lipids. In some aspects, an at least one PEGylated lipid can be a mixture of two PEGylated lipids.

In some aspects, a lipid nanoparticle can comprise at least one nucleic acid molecule, at least one bioreducible ionizable cationic lipid, at least one structural lipid, at least one PEGylated lipid or any combination thereof. In some aspects, an at least one bioreducible ionizable cationic lipid can be ssPalmO-Ph-P4C2.

In some aspects, a lipid nanoparticle can comprise at least one nucleic acid molecule, at least one bioreducible ionizable cationic lipid, at least one structural lipid, and at least one PEGylated lipid. In some aspects, an at least one bioreducible ionizable cationic lipid can be ssPalmO-Ph-P4C2.

In some aspects, a lipid nanoparticle can comprise at least one nucleic acid molecule, at least one bioreducible ionizable cationic lipid, at least one structural lipid, at least one phospholipid, at least one PEGylated lipid or any combination thereof. In some aspects, an at least one bioreducible ionizable cationic lipid can be ssPalmO-Ph-P4C2.

In some aspects, a lipid nanoparticle can comprise at least one nucleic acid molecule, at least one bioreducible ionizable cationic lipid, at least one structural lipid, at least one phospholipid and at least one PEGylated lipid. In some aspects, an at least one bioreducible ionizable cationic lipid can be ssPalmO-Ph-P4C2.

In some aspects, a lipid nanoparticle comprising at least one nucleic acid molecule can comprise about 28% ssPalmO-Ph-P4C2by moles, about 60% cholesterol by moles, about 10% DOPE by moles, and about 2% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 10:1 (w/w). In some aspects, the ratio of lipid to nucleic acid in the at least one nanoparticle can be about 100:1 (w/w).

In some aspects, a lipid nanoparticle comprising at least one nucleic acid molecule can comprise about 60% ssPalmO-Ph-P4C2by moles, about 29.5% cholesterol by moles, about 10% DOPE by moles, and about 0.5% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 32.92% ssPalmO-Ph-P4C2by moles, about 32.92% cholesterol by moles, about 32.92% DOPE by moles, and about 1.25% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 55:1 (w/w).

In some aspects, a lipid nanoparticle comprising at least one nucleic acid molecule can comprise about 10% ssPalmO-Ph-P4C2by moles, about 29.5% cholesterol by moles, about 60% DOPE by moles, and about 0.5% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 60% ssPalmO-Ph-P4C2by moles, between about 28% and 29% cholesterol by moles, about 10% DOPE by moles, and between about 1.25% and 2% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 60% ssPalmO-Ph-P4C2by moles, about 28% cholesterol by moles, about 10% DOPE by moles, and about 2% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 60% ssPalmO-Ph-P4C2by moles, about 28.75% cholesterol by moles, about 10% DOPE by moles, and about 1.25% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2by moles, about 35% cholesterol by moles, about 10% DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 27.84% ssPalmO-Ph-P4C2by moles, about 56.25% cholesterol by moles, about 13.46% DOPE by moles, and about 2.45% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 88:1 (w/w).

In some aspects, a lipid nanoparticle comprising at least one nucleic acid molecule can comprise about 27.9% ssPalmO-Ph-P4C2by moles, about 51.51% cholesterol by moles, about 18.59% DOPE by moles, and about 2% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 60% ssPalmO-Ph-P4C2by moles, about 26.4% cholesterol by moles, about 11.6% DOPE by moles, and about 2% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 70:1 (w/w).

In some aspects, a lipid nanoparticle comprising at least one nucleic acid molecule can comprise about 30.37% ssPalmO-Ph-P4C2by moles, about 37.27% cholesterol by moles, about 30.36% DOPE by moles, and about 2% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise between about 54% to 59% ssPalmO-Ph-P4C2 by moles, between about 30% to 40% cholesterol by moles, between about 5% to 10% of DOPC, DSPC or DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 to about 100:1 (w/w).

In some aspects, a lipid nanoparticle comprising at least one nucleic acid molecule can comprise about 54% ssPalmO-Ph-P4C2 by moles, about 35% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2 by moles, about 35% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 56.5% ssPalmO-Ph-P4C2 by moles, about 32.5% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 56.5% ssPalmO-Ph-P4C2 by moles, about 32.5% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 59% ssPalmO-Ph-P4C2 by moles, about 30% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 59% ssPalmO-Ph-P4C2 by moles, about 30% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 54% ssPalmO-Ph-P4C2 by moles, about 40% cholesterol by moles, about 5% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2 by moles, about 40% cholesterol by moles, about 5% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 56.5% ssPalmO-Ph-P4C2 by moles, about 37.5% cholesterol by moles, about 5% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 56.5% ssPalmO-Ph-P4C2 by moles, about 37.5% cholesterol by moles, about 5% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 59% ssPalmO-Ph-P4C2 by moles, about 35% cholesterol by moles, about 5% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 59% ssPalmO-Ph-P4C2 by moles, about 35% cholesterol by moles, about 5% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 54% ssPalmO-Ph-P4C2 by moles, about 35% cholesterol by moles, about 10% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2 by moles, about 35% cholesterol by moles, about 10% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 56.5% ssPalmO-Ph-P4C2 by moles, about 32.5% cholesterol by moles, about 10% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 56.5% ssPalmO-Ph-P4C2 by moles, about 32.5% cholesterol by moles, about 10% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 59% ssPalmO-Ph-P4C2 by moles, about 30% cholesterol by moles, about 10% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 59% ssPalmO-Ph-P4C2 by moles, about 30% cholesterol by moles, about 10% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 54% ssPalmO-Ph-P4C2 by moles, about 40% cholesterol by moles, about 5% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2 by moles, about 40% cholesterol by moles, about 5% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 56.5% ssPalmO-Ph-P4C2 by moles, about 37.5% cholesterol by moles, about 5% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 56.5% ssPalmO-Ph-P4C2 by moles, about 37.5% cholesterol by moles, about 5% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 59% ssPalmO-Ph-P4C2 by moles, about 35% cholesterol by moles, about 5% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 59% ssPalmO-Ph-P4C2 by moles, about 35% cholesterol by moles, about 5% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 54% ssPalmO-Ph-P4C2 by moles, about 35% cholesterol by moles, about 10% of DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2 by moles, about 35% cholesterol by moles, about 10% of DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 56.5% ssPalmO-Ph-P4C2 by moles, about 32.5% cholesterol by moles, about 10% of DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 56.5% ssPalmO-Ph-P4C2 by moles, about 32.5% cholesterol by moles, about 10% of DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 59% ssPalmO-Ph-P4C2 by moles, about 30% cholesterol by moles, about 10% of DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 59% ssPalmO-Ph-P4C2 by moles, about 30% cholesterol by moles, about 10% of DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 54% ssPalmO-Ph-P4C2 by moles, about 40% cholesterol by moles, about 5% of DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2 by moles, about 40% cholesterol by moles, about 5% of DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 56.5% ssPalmO-Ph-P4C2 by moles, about 37.5% cholesterol by moles, about 5% of DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 56.5% ssPalmO-Ph-P4C2 by moles, about 37.5% cholesterol by moles, about 5% of DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise about 59% ssPalmO-Ph-P4C2 by moles, about 35% cholesterol by moles, about 5% of DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise about 59% ssPalmO-Ph-P4C2 by moles, about 35% cholesterol by moles, about 5% of DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 75:1 (w/w).

In some aspects, a lipid nanoparticle comprising at least one nucleic acid molecule can comprise between about 49% to 60% of ssPalmO-Ph-P4C2 by moles, between about 32% and 44% of cholesterol by moles, about 5% of DOPC by moles, and between about 1.5% and 3.0% of DMG-PEG2000 by moles, wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 40:1 to about 100:1 (w/w).

In some aspects, a lipid nanoparticle comprising at least one nucleic acid molecule can comprise between about 54% to 59% of ssPalmO-Ph-P4C2 by moles; between about 30% to 40% of cholesterol by moles, about 5% of DOPC by moles, and about 1% of DMG-PEG2000 by moles, wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 75:1 to about 100:1 (w/w).

In some aspects, a lipid nanoparticle comprising at least one nucleic acid molecule can comprise about 54% of ssPalmO-Ph-P4C2 by moles; about 40% of cholesterol by moles, about 5% of DOPC by moles, and about 1% of DMG-PEG2000 by moles, wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 75:1 to about 100:1 (w/w).

In some aspects, a lipid nanoparticle comprising at least one nucleic acid molecule can comprise about 56.5% of ssPalmO-Ph-P4C2 by moles; about 37.5% of cholesterol by moles, about 5% of DOPC by moles, and about 1% of DMG-PEG2000 by moles, wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 75:1 to about 100:1 (w/w).

In some aspects, a lipid nanoparticle comprising at least one nucleic acid molecule can comprise about 59% of ssPalmO-Ph-P4C2 by moles; about 30% of cholesterol by moles, about 5% of DOPC by moles, and about 1% of DMG-PEG2000 by moles, wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 75:1 to about 100:1 (w/w).

In some aspects, a lipid nanoparticle comprising at least one nucleic acid molecule can comprise about 49.4% ssPalmO-Ph-P4C2by moles, about 44% cholesterol by moles, about 5% of DOPC by moles, and about 1.6% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 60:1 (w/w).

In some aspects, a lipid nanoparticle comprising at least one nucleic acid molecule can comprise about 60% ssPalmO-Ph-P4C2by moles, about 32.8% cholesterol by moles, about 5% of DSPC by moles, and about 2.2% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 60:1 (w/w).

In some aspects, a lipid nanoparticle comprising at least one nucleic acid molecule can comprise about 60% ssPalmO-Ph-P4C2by moles, about 34% cholesterol by moles, about 5% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 40:1 (w/w).

In some aspects, a lipid nanoparticle comprising at least one nucleic acid molecule can comprise about 41.8% ssPalmO-Ph-P4C2by moles, about 52.2% cholesterol by moles, about 5% of DSPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 60:1 (w/w).

In some aspects, the nucleic acid molecule is a DNA molecule. Thus, the present disclosure provides a lipid nanoparticle comprising at least one nucleic acid molecule can comprise about 22.71% ssPalmO-Ph-P4C2by moles, about 55.21% cholesterol by moles, about 20.89% DOPE by moles, and about 1.25% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA (see WO/2020/154645). In some aspects, the ratio of lipid to nucleic acid in the nanoparticle can be about 100:1 (w/w).

In some aspects, a lipid nanoparticle comprising at least one nucleic acid molecule can comprise about 50% ssPalmO-Ph-P4C2by moles, about 38.6% cholesterol by moles, about 10% DOPE by moles, and about 1.4% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA (see WO/2020/154645). In some aspects, the ratio of lipid to nucleic acid in the nanoparticle can be about 200:1 (w/w).

In some aspects, a lipid nanoparticle comprising at least one nucleic acid molecule can comprise about 34.69% ssPalmO-Ph-P4C2by moles, about 39.74% cholesterol by moles, about 24.69% DOPE by moles, and about 1.25% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the nanoparticle can be about 50:1 (w/w).

In some aspects, a lipid nanoparticle comprising at least one nucleic acid molecule can comprise about 50% ssPalmO-Ph-P4C2by moles, about 20% cholesterol by moles, about 29.5% DOPE by moles, and about 0.5% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the nanoparticle can be about 100:1 (w/w).

In some aspects, a lipid nanoparticle comprising at least one nucleic acid molecule can comprise about 50% of ssPalmO-Ph-P4C2 by moles, about 20% of cholesterol by moles, about 28.7% of DOPE by moles, and about 1.3% of DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the nanoparticle can be about 50:1 (w/w).

In some aspects, a lipid nanoparticle comprising at least one nucleic acid molecule can comprise between about 54% to 59% ssPalmO-Ph-P4C2 by moles, between about 30% to 40% cholesterol by moles, between about 5% to 10% of DOPC, DSPC or DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 to about 100:1 (w/w).

In some aspects, a lipid nanoparticle comprising at least one nucleic acid molecule can comprise about 54% ssPalmO-Ph-P4C2 by moles, about 35% cholesterol by moles, about 10% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2 by moles, about 35% cholesterol by moles, about 10% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 56.5% ssPalmO-Ph-P4C2 by moles, about 32.5% cholesterol by moles, about 10% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 56.5% ssPalmO-Ph-P4C2 by moles, about 32.5% cholesterol by moles, about 10% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 59% ssPalmO-Ph-P4C2 by moles, about 30% cholesterol by moles, about 10% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 59% ssPalmO-Ph-P4C2 by moles, about 30% cholesterol by moles, about 10% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 54% ssPalmO-Ph-P4C2 by moles, about 40% cholesterol by moles, about 5% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2 by moles, about 40% cholesterol by moles, about 5% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 56.5% ssPalmO-Ph-P4C2 by moles, about 37.5% cholesterol by moles, about 5% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 56.5% ssPalmO-Ph-P4C2 by moles, about 37.5% cholesterol by moles, about 5% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 59% ssPalmO-Ph-P4C2 by moles, about 35% cholesterol by moles, about 5% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 59% ssPalmO-Ph-P4C2 by moles, about 35% cholesterol by moles, about 5% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 54% ssPalmO-Ph-P4C2 by moles, about 35% cholesterol by moles, about 10% of DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2 by moles, about 35% cholesterol by moles, about 10% of DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 56.5% ssPalmO-Ph-P4C2 by moles, about 32.5% cholesterol by moles, about 10% of DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 56.5% ssPalmO-Ph-P4C2 by moles, about 32.5% cholesterol by moles, about 10% of DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 59% ssPalmO-Ph-P4C2 by moles, about 30% cholesterol by moles, about 10% of DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 59% ssPalmO-Ph-P4C2 by moles, about 30% cholesterol by moles, about 10% of DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 54% ssPalmO-Ph-P4C2 by moles, about 40% cholesterol by moles, about 5% of DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2 by moles, about 40% cholesterol by moles, about 5% of DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 56.5% ssPalmO-Ph-P4C2 by moles, about 37.5% cholesterol by moles, about 5% of DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 56.5% ssPalmO-Ph-P4C2 by moles, about 37.5% cholesterol by moles, about 5% of DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 59% ssPalmO-Ph-P4C2 by moles, about 35% cholesterol by moles, about 5% of DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 59% ssPalmO-Ph-P4C2 by moles, about 35% cholesterol by moles, about 5% of DSPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 54% ssPalmO-Ph-P4C2 by moles, about 35% cholesterol by moles, about 10% of DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2 by moles, about 35% cholesterol by moles, about 10% of DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 56.5% ssPalmO-Ph-P4C2 by moles, about 32.5% cholesterol by moles, about 10% of DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 56.5% ssPalmO-Ph-P4C2 by moles, about 32.5% cholesterol by moles, about 10% of DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 59% ssPalmO-Ph-P4C2 by moles, about 30% cholesterol by moles, about 10% of DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 59% ssPalmO-Ph-P4C2 by moles, about 30% cholesterol by moles, about 10% of DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 54% ssPalmO-Ph-P4C2 by moles, about 40% cholesterol by moles, about 5% of DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 54% ssPalmO-Ph-P4C2 by moles, about 40% cholesterol by moles, about 5% of DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 56.5% ssPalmO-Ph-P4C2 by moles, about 37.5% cholesterol by moles, about 5% of DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 56.5% ssPalmO-Ph-P4C2 by moles, about 37.5% cholesterol by moles, about 5% of DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise about 59% ssPalmO-Ph-P4C2 by moles, about 35% cholesterol by moles, about 5% of DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 100:1 (w/w).

In some aspects, a lipid nanoparticle comprising at least one nucleic acid molecule can comprise about 59% ssPalmO-Ph-P4C2 by moles, about 35% cholesterol by moles, about 5% of DOPE by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 (w/w).

In some aspects, a lipid nanoparticle comprising at least one nucleic acid molecule can comprise between about 48% to about 61% of ssPalmO-Ph-P4C2 by moles, between about 31% to about 53% of cholesterol by moles, between about 4% to about 11% of phospholipid by moles, and about 0.5% to about 3% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 30:1 to about 110:1 (w/w). In some aspects, the ratio of lipid to nucleic acid can be about 40:1 (w/w). In some aspects, the ratio of lipid to nucleic acid can be about 60:1 (w/w). In some aspects, the ratio of lipid to nucleic acid can be about 100:1 (w/w).

In some aspects, a lipid nanoparticle comprising at least one nucleic acid molecule can comprise between about 41.8% to about 60% of ssPalmO-Ph-P4C2 by moles, between about 32.8% to about 52.2% of cholesterol by moles, between about 5% to about 10% of phospholipid by moles, and about 1% to about 2.2% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 30:1 to about 110:1 (w/w). In some aspects, the ratio of lipid to nucleic acid can be about 40:1 (w/w). In some aspects, the ratio of lipid to nucleic acid can be about 60:1 (w/w). In some aspects, the ratio of lipid to nucleic acid can be about 100:1 (w/w).

In some aspects, a lipid nanoparticle comprising at least one nucleic acid molecule can comprise about 54% of ssPalmO-Ph-P4C2 by moles, about 35% of cholesterol by moles, about 10% of phospholipid by moles, and about 1% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 30:1 to about 110:1 (w/w). In some aspects, the ratio of lipid to nucleic acid can be about 40:1 (w/w). In some aspects, the ratio of lipid to nucleic acid can be about 60:1 (w/w). In some aspects, the ratio of lipid to nucleic acid can be about 100:1 (w/w).

In some aspects, a lipid nanoparticle comprising at least one nucleic acid molecule can comprise about 49.4% of ssPalmO-Ph-P4C2 by moles, about 44% of cholesterol by moles, about 5% of phospholipid by moles, and about 1.6% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 30:1 to about 110:1 (w/w). In some aspects, the ratio of lipid to nucleic acid can be about 40:1 (w/w). In some aspects, the ratio of lipid to nucleic acid can be about 60:1 (w/w). In some aspects, the ratio of lipid to nucleic acid can be about 100:1 (w/w).

In some aspects, a lipid nanoparticle comprising at least one nucleic acid molecule can comprise about 60% of ssPalmO-Ph-P4C2 by moles, about 32.8% of cholesterol by moles, about 5% of phospholipid by moles, and about 2.2% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 30:1 to about 110:1 (w/w). In some aspects, the ratio of lipid to nucleic acid can be about 40:1 (w/w). In some aspects, the ratio of lipid to nucleic acid can be about 60:1 (w/w). In some aspects, the ratio of lipid to nucleic acid can be about 100:1 (w/w).

In some aspects, a lipid nanoparticle comprising at least one nucleic acid molecule can comprise about 60% of ssPalmO-Ph-P4C2 by moles, about 34% of cholesterol by moles, about 5% of phospholipid by moles, and about 1% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 30:1 to about 110:1 (w/w). In some aspects, the ratio of lipid to nucleic acid can be about 40:1 (w/w). In some aspects, the ratio of lipid to nucleic acid can be about 60:1 (w/w). In some aspects, the ratio of lipid to nucleic acid can be about 100:1 (w/w).

In some aspects, a lipid nanoparticle comprising at least one nucleic acid molecule can comprise about 41.8% of ssPalmO-Ph-P4C2 by moles, about 52.2% of cholesterol by moles, about 5% of phospholipid by moles, and about 1% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 30:1 to about 110:1 (w/w). In some aspects, the ratio of lipid to nucleic acid can be about 40:1 (w/w). In some aspects, the ratio of lipid to nucleic acid can be about 60:1 (w/w). In some aspects, the ratio of lipid to nucleic acid can be about 100:1 (w/w).

In some aspects, a lipid nanoparticle comprising at least one nucleic acid molecule can comprise about 54% of ssPalmO-Ph-P4C2 by moles, about 35% of cholesterol by moles, about 10% of phospholipid by moles, and about 1% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 30:1 to about 110:1 (w/w). In some aspects, the ratio of lipid to nucleic acid can be about 40:1 (w/w). In some aspects, the ratio of lipid to nucleic acid can be about 60:1 (w/w). In some aspects, the ratio of lipid to nucleic acid can be about 100:1 (w/w).

In some aspects, a lipid nanoparticle comprising at least one nucleic acid molecule can comprise between about 53% to about 60% of ssPalmO-Ph-P4C2 by moles, between about 34% to about 41% of cholesterol by moles, between about 4% to about 11% of phospholipid by moles, and about 0.5% to about 2% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 70:1 to about 105:1 (w/w). In some aspects, the ratio of lipid to nucleic acid can be about 75:1 (w/w). In some aspects, the ratio of lipid to nucleic acid can be about 100:1 (w/w).

In some aspects, a lipid nanoparticle comprising at least one nucleic acid molecule can comprise between about 54% to about 59% of ssPalmO-Ph-P4C2 by moles, between about 35% to about 40% of cholesterol by moles, between about 5% to about 10% of phospholipid by moles, and about 0.5% to about 1.5% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 70:1 to about 105:1 (w/w). In some aspects, the ratio of lipid to nucleic acid can be about 75:1 (w/w). In some aspects, the ratio of lipid to nucleic acid can be about 100:1 (w/w).

In some aspects, a lipid nanoparticle comprising at least one nucleic acid molecule can comprise about 54% of ssPalmO-Ph-P4C2 by moles, about 35% of cholesterol by moles, about 10% of phospholipid by moles, and about 1% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 70:1 to about 105:1 (w/w). In some aspects, the ratio of lipid to nucleic acid can be about 75:1 (w/w). In some aspects, the ratio of lipid to nucleic acid can be about 100:1 (w/w).

In some aspects, a lipid nanoparticle comprising at least one nucleic acid molecule can comprise about 59% of ssPalmO-Ph-P4C2 by moles, about 35% of cholesterol by moles, about 5% of phospholipid by moles, and about 1% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 70:1 to about 105:1 (w/w). In some aspects, the ratio of lipid to nucleic acid can be about 75:1 (w/w). In some aspects, the ratio of lipid to nucleic acid can be about 100:1 (w/w).

In some aspects, a lipid nanoparticle comprising at least one nucleic acid molecule can comprise about 54% of ssPalmO-Ph-P4C2 by moles, about 40% of cholesterol by moles, about 5% of phospholipid by moles, and about 1% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 70:1 to about 105:1 (w/w). In some aspects, the ratio of lipid to nucleic acid can be about 75:1 (w/w). In some aspects, the ratio of lipid to nucleic acid can be about 100:1 (w/w).

In some aspects, a lipid nanoparticle comprising at least one nucleic acid molecule can comprise about 56.5% of ssPalmO-Ph-P4C2 by moles, about 37.5% of cholesterol by moles, about 5% of phospholipid by moles, and about 1% of DMG-PEG2000 by moles. In some aspects, the at least one nucleic acid molecule comprises at least one RNA molecule. In some aspects, the at least one RNA molecule can be mRNA. In some aspects, the mRNA molecule can further comprise a 5′-CAP. In some aspects, the at least one nucleic acid molecule can comprise at least one DNA molecule. In some aspects, the at least one DNA molecule can be DoggyBone DNA or a DNA nanoplasmid. In some aspects, the phospholipid can be DOPE. In some aspects, the phospholipid can be DOPC. In some aspects, the phospholipid can be DSPC. In some aspects, the ratio of lipid to nucleic acid in the nanoparticle can be about 70:1 to about 105:1 (w/w). In some aspects, the ratio of lipid to nucleic acid can be about 75:1 (w/w). In some aspects, the ratio of lipid to nucleic acid can be about 100:1 (w/w).

In some aspects of the present disclosure, a lipid nanoparticle, or a plurality of lipid nanoparticles, can be stable at about 2° C. to about 6° C., or about 4° C., for at least about 1 day, about 2 days, about 3 days, about 4 days, about 5 days, about 6 days, about 7 days, about 8 days, about 9 days, about 10 days, about 11 days, about 12 days, about 13 days or about 14 days. In some aspects of the present disclosure, a lipid nanoparticle can be stable at about 4° C., for at least about 7 days.

In some aspects of the present disclosure, a lipid nanoparticle, or a plurality of lipid nanoparticles can be stable at about 2° C. to about 6° C., or about 4° C., for about 1 day, about 2 days, about 3 days, about 4 days, about 5 days, about 6 days, about 7 days, about 8 days, about 9 days, about 10 days, about 11 days, about 12 days, about 13 days or about 14 days. In some aspects of the present disclosure, a lipid nanoparticle, or a plurality of lipid nanoparticles, can be stable at about 4° C., for about 7 days.

In some aspects, a lipid nanoparticle, or a plurality of lipid nanoparticle, can be said to be stable if there is no more than about a 0.5%, or about a 1%, or about a 5%, or about a 10%, or about a 15%, or about a 20%, or about a 25% chance, or about a 30%, or about a 35%, or about a 40%, or about a 45%, or about a 50% change in the diameter (in the case of a single lipid nanoparticle) or mean diameter (in the case of a plurality of lipid nanoparticles). The diameter of a lipid nanoparticle or the mean diameter of a plurality of nanoparticles can be determined using any standard method known in the art, as would be appreciated by the skilled artisan.

In some aspects, a plurality of lipid nanoparticle can be said to be stable if there is no more than about a 0.5%, or about a 1%, or about a 5%, or about a 10%, or about a 15%, or about a 20%, or about a 25% chance, or about a 30%, or about a 35%, or about a 40%, or about a 45%, or about a 50% change in the polydispersity index (PDI) or the plurality of lipid nanoparticles. The PDI of a plurality of lipid nanoparticles can be determined using any standard method known in the art, as would be appreciated by the skilled artisan.

C12-200 LNPs of the Present Disclosure

The present disclosure also provides lipid nanoparticles comprising at least one nucleic acid molecule, at least one lipidoid, at least one structural lipid, at least one phospholipid, at least one PEGylated lipid or any combination thereof.

The lipidoid can be C12-200, also referred to as 1,1′-((2-(4-(2-42-(bis(2-hydroxydodecyl)amino)ethyl) (2-hydroxydodecyl)amino)ethyl)piperazin-1-yl)ethyl)azanediyl)bis(dodecan-2-ol), hereafter referred to as “C12-200” (see U.S. Pat. Nos. 8,450,298, 8,969,353, 9,556,110 and 10,189,802. See also US Patent Publication No. 2019-0177289).

Accordingly, the lipid nanoparticles comprising at least one nucleic acid molecule of the present disclosure can comprise about 25% to about 45% of C12-200 by moles, about 32% to about 52% of at least one structural lipid by moles, about 10% to about 30% of at least one phospholipid by moles, and about 0.1% to about 13% of at least one PEGylated lipid by moles. In some aspects, a lipid nanoparticle comprising at least one nucleic acid molecule can comprise about 30% to about 40% of C12-200 by moles, about 37% to about 47% of at least one structural lipid by moles, about 15% to about 25% of at least one phospholipid by moles, and about 0.1% to about 8% of at least one PEGylated lipid by moles. In some aspects, a lipid nanoparticle comprising at least one nucleic acid molecule can comprise about 32.5% to about 37.5% of C12-200 moles, about 39.5% to about 44.5% of at least one structural lipid by moles, about 17.5% to about 22.5% of at least one phospholipid by moles, and about 0.5% to about 5.5% of at least one PEGylated lipid by moles. In some aspects, a lipid nanoparticle comprising at least one nucleic acid molecule can comprise about 35% of C12-200 by moles, about 42% of at least one structural lipid by moles, about 20% of at least one phospholipid by moles, and about 3% of at least one PEGylated lipid by moles. In some aspects, a lipid nanoparticle comprising at least one nucleic acid molecule can comprise about 35% of C12-200 by moles, about 41.84% of at least one structural lipid by moles, about 20% of at least one phospholipid by moles, and about 3.16% of at least one PEGylated lipid by moles. In some aspects, the preceding lipid nanoparticles can comprise lipid and nucleic acid at a ratio of about 60:1 to about 100:1, or about 70:1 to about 90:1, or about 75:1 to about 85:1, lipid:nucleic acid, weight/weight. In some aspects, the preceding lipid nanoparticles can comprise lipid and nucleic acid at a ratio of about 80:1, lipid:nucleic acid, weight/weight. The phospholipid can be DOPE. The phospholipid can be DPSC. The phospholipid can be DOPC. The structural lipid can be cholesterol. The PEGylated lipid can be DMG-PEG2000.

In some aspects, the at least one nucleic acid molecule is a DNA molecule or an RNA molecule. In some aspects, the nucleic acid molecule is a DNA molecule (e.g. a DoggyBone DNA molecule). Thus, the present disclosure provides a lipid nanoparticle comprising about 35% of C12-200 by moles, about 42% of at least one structural lipid by moles, about 20% of at least one phospholipid by moles, and about 3% of at least one PEGylated lipid by moles, wherein the at least one nucleic acid comprises at least one DNA molecule. The present disclosure also provides a lipid nanoparticle comprising about 35% of C12-200 by moles, about 41.84% of at least one structural lipid by moles, about 20% of at least one phospholipid by moles, and about 3.16% of at least one PEGylated lipid by moles, wherein the at least one nucleic acid comprises at least one DNA molecule. In some aspects, the at least one DNA molecule is a DoggyBone DNA molecule. In some aspects, the at least one DNA molecule is a DNA nanoplasmid. In some aspects, the lipid nanoparticle comprising at least one nucleic acid molecule can comprise about 35% of at least one titratable cationic lipid by moles, about 42% of at least one structural lipid by moles, about 20% of at least one phospholipid by moles, and about 3% of at least one PEGylated lipid by moles, wherein the at least one nucleic acid is a DNA molecule (e.g. a DoggyBone DNA molecule, a DNA nanoplasmid), wherein the ratio of lipid to nucleic acid in the nanoparticle is about 80:1 (weight/weight). In some aspects, the lipid nanoparticle comprising at least one nucleic acid molecule can comprise about 35% of at least one titratable cationic lipid by moles, about 41.84% of at least one structural lipid by moles, about 20% of at least one phospholipid by moles, and about 3.16% of at least one PEGylated lipid by moles, wherein the at least one nucleic acid is a DNA molecule (e.g. a DoggyBone DNA molecule, A DNA nanoplasmid), wherein the ratio of lipid to nucleic acid in the nanoparticle is about 80:1 (weight/weight). In some aspects, the preceding lipid nanoparticles can be used for the delivery of at least one DNA molecule (e.g. a DoggyBone DNA molecule, a DNA nanoplasmid) to at least one liver cell. The phospholipid can be DOPE. The phospholipid can be DPSC. The phospholipid can be DOPC. The structural lipid can be cholesterol. The PEGylated lipid can be DMG-PEG2000.

Alternative LNP Embodiments of the Present Disclosure
1. A composition comprising at least one lipid nanoparticle comprising:
about 23% to about 33% of ssPalmO-Ph-P4C2 by moles;
about 55% to about 65% of cholesterol by moles,
about 5% to about 15% of DOPE by moles, and
about 0.01% to about 7% of DMG-PEG2000 by moles.
wherein the at least one lipid nanoparticle comprises at least one nucleic acid molecule, wherein the at least one nucleic acid molecule comprises at least one RNA molecule.
2. The composition of embodiment 1, wherein the at least one lipid nanoparticle comprises:
about 25.5% to about 30.5% of ssPalmO-Ph-P4C2 by moles;
about 57.5% to about 62.5% of cholesterol by moles,
about 7.5% to about 12.5% of DOPE by moles, and
about 0.1% to about 4.5% of DMG-PEG2000 by moles.
3. The composition of embodiment 1 or embodiment 2, wherein the at least one lipid nanoparticle comprises:
about 27% to about 29% of ssPalmO-Ph-P4C2 by moles;
about 59% to about 61% of cholesterol by moles,
about 9% to about 11% of DOPE by moles, and
about 1% to about 3% of DMG-PEG2000 by moles.
4. The composition of any one of embodiments 1-3, wherein the at least one lipid nanoparticle comprises:
about 28% of ssPalmO-Ph-P4C2 by moles;
about 60% of cholesterol by moles,
about 10% of DOPE by moles, and
about 2% of DMG-PEG2000 by moles.
5. The composition of any one of embodiments 1-4, wherein the ratio of lipid to nucleic acid in the at least one lipid nanoparticle is about 5:1 to about 15:1 (w/w)
6. The composition of any one of embodiments 1-5, wherein the ratio of lipid to nucleic acid in the at least one lipid nanoparticle is about 10:1 (w/w).
7. The composition of any one of embodiments 1-6, wherein the ratio of lipid to nucleic acid in that least one lipid nanoparticle is about 95:1 to about 105:1 (w/w).
8. The composition of any one of embodiments 1-7, wherein the ratio of lipid to nucleic acid in the at least one nanoparticle is about 100:1 (w/w).
9. The composition of embodiments 1-8, wherein the at least one lipid nanoparticle comprises:
about 28% of ssPalmO-Ph-P4C2 by moles;
about 60% of cholesterol by moles,
about 10% of DOPE by moles, and
about 2% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one lipid nanoparticle is about 10:1 (w/w).
10. The composition of any one of embodiments 1-9, wherein the at least one lipid nanoparticle comprises:
about 28% of ssPalmO-Ph-P4C2 by moles;
about 60% of cholesterol by moles,
about 10% of DOPE by moles, and
about 2% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one lipid nanoparticle is about 100:1 (w/w).
11. A composition comprising at least one lipid nanoparticle comprising:
about 55% to about 65% of ssPalmO-Ph-P4C2 by moles,
about 24.5% to about 34.5% of cholesterol by moles,
about 5% to about 15% of DOPE by moles, and
about 0.01% to about 5.5% of DMG-PEG2000 by moles,
wherein the at least one lipid nanoparticle comprises at least one nucleic acid molecule, wherein the at least one nucleic acid molecule comprises at least one RNA molecule.
12. The composition of embodiment 11, wherein the at least one lipid nanoparticle comprises:
about 57.5% to about 62.5% of ssPalmO-Ph-P4C2 by moles,
about 27% to about 32% of cholesterol by moles,
about 7.5% to about 12.5% of DOPE by moles, and
about 0.1% to about 3% of DMG-PEG2000 by moles.
13. The composition of embodiment 11 or embodiment 12, wherein the at least one lipid nanoparticle comprises:
about 59% to about 61% of ssPalmO-Ph-P4C2 by moles,
about 28.5% to about 30.5% of cholesterol by moles,
about 9% to about 11% of DOPE by moles, and
about 0.1% to about 1.5% of DMG-PEG2000 by moles.
14. The composition of any one of embodiments 11-13, wherein the at least lone lipid nanoparticle comprises:
about 60% of ssPalmO-Ph-P4C2 by moles, about 29.5% of cholesterol by moles, about 10% of DOPE by moles, and about 0.5% of DMG-PEG2000 by moles.
15. The composition of any one of embodiments 11-14, wherein the ratio of lipid to nucleic acid in the at least one lipid nanoparticle is about 95:1 to about 105:1 (w/w).
16. The composition of any one of embodiments 11-15, wherein the ratio of lipid to nucleic acid in the at least one lipid nanoparticle is about 100:1 (w/w).
17. The composition of any one of embodiments 11-16, wherein the at least one nanoparticle comprises:
about 60% of Coatsome SS-OP by moles,
about 29.5% of cholesterol by moles,
about 10% of DOPE by moles, and
about 0.5% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one lipid nanoparticle is about 100:1 (w/w).
18. A composition comprising at least one lipid nanoparticle comprising:
about 27.92% to about 37.92% of ssPalmO-Ph-P4C2 by moles,
about 27.92% to about 37.92% of cholesterol by moles,
about 27.92% to about 37.92% of DOPE by moles, and
about 0.01% to about 6.25% of DMG-PEG2000 by moles,
wherein the at least one lipid nanoparticle comprises at least one nucleic acid molecule, wherein the at least one nucleic acid molecule comprises at least one RNA molecule.
19. The composition of embodiment 18, wherein the at least one lipid nanoparticle comprises:
about 30.42% to about 35.42% of ssPalmO-Ph-P4C2 by moles,
about 30.42% to about 35.42% of cholesterol by moles,
about 30.42% to about 35.42% of DOPE by moles, and
about 0.1% to about 3.75% of DMG-PEG2000 by moles.
20. The composition of embodiment 18 or embodiment 19, wherein the at least one lipid nanoparticle comprises:
about 31.92% to about 32.92% of ssPalmO-Ph-P4C2 by moles, about 31.92% to about 32.92% of cholesterol by moles, about 31.92% to about 32.92% of DOPE by moles, and about 0.25% to about 2.25% of DMG-PEG2000 by moles.
21. The composition of any one of embodiments 18-20, wherein the at least one lipid nanoparticle comprises:
about 32.92% of ssPalmO-Ph-P4C2 by moles, about 32.92% of cholesterol by moles, about 32.92% of DOPE by moles, and about 1.25% of DMG-PEG2000 by moles.
22. The composition of any one of embodiments 18-21, wherein the ratio of lipid to nucleic acid in the at least one lipid nanoparticle is about 50:1 to about 60:1 (w/w).
23. The composition of any one of embodiments 18-22, wherein the ratio of lipid to nucleic acid in the at least one lipid nanoparticle is about 55:1 (w/w).
24. The composition of any one of embodiments 18-23, wherein the at least one lipid nanoparticle comprises:
about 32.92% of ssPalmO-Ph-P4C2 by moles,
about 32.92% of cholesterol by moles,
about 32.92% of DOPE by moles, and
about 1.25% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one lipid nanoparticle is about 55:1 (w/w).
25. A composition comprising at least one lipid nanoparticle comprising:
about 5% to about 15% of ssPalmO-Ph-P4C2 by moles,
about 24.5% to about 34.5% of cholesterol by moles,
about 55% to about 65% of DOPE by moles, and
about 0.01% to about 5.5% of DMG-PEG2000 by moles,
wherein the at least one lipid nanoparticle comprises at least one nucleic acid molecule, wherein the at least one nucleic acid molecule comprises at least one RNA molecule.
26. The composition of embodiment 25, wherein the at least one lipid nanoparticle comprises:
about 7.5% to about 12.5% of ssPalmO-Ph-P4C2 by moles,
about 27% to about 32% of cholesterol by moles,
about 57.5% to about 62.5% of DOPE by moles, and
about 0.1% to about 3% of DMG-PEG2000 by moles.
27. The composition of embodiment 25 or embodiment 26, wherein the at least one lipid nanoparticle comprises:
about 9% to about 11% of ssPalmO-Ph-P4C2 by moles,
about 28.5% to about 30.5% of cholesterol by moles,
about 59% to about 61% of DOPE by moles, and
about 0.1% to about 1.5% of DMG-PEG2000 by moles.
28. The composition of any one of embodiments 25-27, wherein the at least one lipid nanoparticle comprises:
about 10% of ssPalmO-Ph-P4C2 by moles,
about 29.5% of cholesterol by moles,
about 60% of DOPE by moles, and
about 0.5% of DMG-PEG2000 by moles.
29. The composition of any one of embodiments 25-28, wherein the ratio of lipid to nucleic acid in the at least one lipid nanoparticle is about 95:1 to about 105:1 (w/w).
30. The composition of any one of embodiments 25-29, wherein the ratio of lipid to nucleic acid in the at least one lipid nanoparticle is about 100:1.
31. The composition of any one of embodiments 25-30, wherein the at least one lipid nanoparticle comprises:
about 10% of ssPalmO-Ph-P4C2 by moles,
about 29.5% of cholesterol by moles,
about 60% of DOPE by moles, and
about 0.5% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one lipid nanoparticle is about 100:1 (w/w).
32. A composition comprising at least one lipid nanoparticle comprising:
about 55% to about 65% of ssPalmO-Ph-P4C2 by moles,
about 23% to about 33% of cholesterol by moles,
about 5% to about 15% of DOPE by moles, and
between about 0.1% to about 7% of DMG-PEG2000 by moles,
wherein the at least one lipid nanoparticle comprises at least one nucleic acid molecule, wherein the at least one nucleic acid molecule comprises at least one RNA molecule.
33. The composition of embodiment 32, wherein the at least one lipid nanoparticle comprises:
about 57.5% to about 62.5% of ssPalmO-Ph-P4C2 by moles,
about 25.5% to about 30.5% of cholesterol by moles,
about 7.5% to about 12.5% of DOPE by moles, and
about 0.1% to about 4.5% of DMG-PEG2000 by moles.
34. The composition of embodiment 32 or embodiment 33, wherein the at least one lipid nanoparticle comprises:
about 59% to about 61% of ssPalmO-Ph-P4C2 by moles,
about 27% to about 29% of cholesterol by moles,
about 9% to about 11% of DOPE by moles, and
between about 1% to about 3% of DMG-PEG2000 by moles.
35. The composition of any one of embodiments 32-34, wherein the at least one lipid nanoparticle comprises:
about 60% of ssPalmO-Ph-P4C2 by moles,
about 28% of cholesterol by moles,
about 10% of DOPE by moles, and
about 2% of DMG-PEG2000 by moles.
36. The composition of any one of embodiments 32-35, wherein the ratio of lipid to nucleic acid in the at least one lipid nanoparticle is about 95:1 to about 105:1 (w/w).
37. The composition of any one of embodiments 32-36, wherein the ratio of lipid to nucleic acid in the at least one lipid nanoparticle is about 100:1 (w/w).
38. The composition of any one of embodiments 32-27, wherein the at least one lipid nanoparticle comprises:
about 60% of ssPalmO-Ph-P4C2 by moles,
about 28% of cholesterol by moles,
about 10% of DOPE by moles, and
about 2% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one lipid nanoparticle is about 100:1 (w/w).
39. A composition comprising at least one lipid nanoparticle comprising:
about 55% to about 65% of ssPalmO-Ph-P4C2 by moles,
about 23.75% to about 33.75% of cholesterol by moles,
about 5% to about 15% of DOPE by moles, and
between about 0.01% to about 6.25% of DMG-PEG2000 by moles,
wherein the at least one lipid nanoparticle comprises at least one nucleic acid molecule, wherein the at least one nucleic acid molecule comprises at least one RNA molecule.
40. The composition of embodiment 39, wherein the at least one nanoparticle comprises:
about 57.5% to about 62.5% of ssPalmO-Ph-P4C2 by moles,
about 26.25% to about 31.25% of cholesterol by moles,
about 7.5% to about 12.5% of DOPE by moles, and
about 0.1% to about 3.75% of DMG-PEG2000 by moles.
41. The composition of embodiment 39 or embodiment 40, wherein the at least one nanoparticle comprises:
about 59% to about 61% of ssPalmO-Ph-P4C2 by moles,
about 27.75% to about 29.75% of cholesterol by moles,
about 9% to about 11% of DOPE by moles, and
about 0.25% to about 2.25% of DMG-PEG2000 by moles.
42. The composition of any one of embodiments 39-41, wherein the at least one nanoparticle comprises:
about 60% of ssPalmO-Ph-P4C2 by moles,
about 28.75% of cholesterol by moles,
about 10% of DOPE by moles, and
about 1.25% of DMG-PEG2000 by moles.
43. The composition of any one of embodiments 39-42, wherein the ratio of lipid to nucleic acid in the at least one nanoparticle is about 95:1 to about 105:1 (w/w).
44. The composition of any one of embodiments 39-43, wherein the ratio of lipid to nucleic acid in the at least one nanoparticle is about 100:1 (w/w).
45. The composition of any one of embodiments 39-44, wherein the at least one lipid nanoparticle comprises:
about 60% of ssPalmO-Ph-P4C2 by moles,
about 28.75% of cholesterol by moles,
about 10% of DOPE by moles, and
about 1.25% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).
46. A composition comprising at least one lipid nanoparticle comprising:
about 17.71% to about 27.71% of ssPalmO-Ph-P4C2 by moles,
about 50.21% to about 60.21% of cholesterol by moles,
about 23.83% to about 33.83% of DOPE by moles, and
about 0.01% to about 6.25% of DMG-PEG2000 by moles,
wherein the at least one lipid nanoparticle comprises at least one nucleic acid molecule, wherein the at least one nucleic acid molecule comprises at least one DNA molecule.
47. The composition of embodiment 46, wherein the at least one lipid nanoparticle comprises:
about 20.21% to about 25.21% of ssPalmO-Ph-P4C2 by moles,
about 52.71% to about 57.71% of cholesterol by moles,
about 26.33% to about 31.33% of DOPE by moles, and
about 0.1% to about 3.75% of DMG-PEG2000 by moles.
48. The composition of embodiment 46 or embodiment 47, wherein the at least one lipid nanoparticle comprises:
about 21.71% to about 23.71% of ssPalmO-Ph-P4C2 by moles,
about 54.21% to about 56.21% of cholesterol by moles,
about 27.83% to about 29.83% of DOPE by moles, and
about 0.25% to about 2.25% of DMG-PEG2000 by moles.
49. The composition of any one of embodiments 46-48, wherein the at least one lipid nanoparticle comprises:
about 22.71% of ssPalmO-Ph-P4C2 by moles,
about 55.21% of cholesterol by moles,
about 28.83% of DOPE by moles, and
about 1.25% of DMG-PEG2000 by moles.
50. The composition of any one of embodiments 46-49, wherein the ratio of lipid to nucleic acid in the at least one lipid nanoparticle is about 95:1 to about 105:1 (w/w).
51. The composition of any one of embodiments 46-50, wherein the ratio of lipid to nucleic acid in the at least one lipid nanoparticle is about 100:1 (w/w).
52. The composition of any one of embodiments 46-51, wherein the at least one lipid nanoparticle comprises:
about 22.71% of ssPalmO-Ph-P4C2 by moles,
about 55.21% of cholesterol by moles,
about 28.83% of DOPE by moles, and
about 1.25% of DMG-PEG2000 by moles,
wherein the at least one lipid nanoparticle comprises at least one nucleic acid molecule, wherein the at least one nucleic acid molecule comprises at least one DNA molecule, and wherein the ratio of lipid to nucleic acid in the at least one nanoparticle is about 100:1 (w/w).
53. A composition comprising at least one lipid nanoparticle comprising:
about 45% to about 55% of ssPalmO-Ph-P4C2 by moles,
about 33.6% to about 43.6% of cholesterol by moles,
about 5% to about 15% of DOPE by moles, and
about 0.01% to about 6.4% of DMG-PEG2000 by moles,
wherein the at least one lipid nanoparticle comprises at least one nucleic acid molecule, wherein the at least one nucleic acid molecule comprises at least one DNA molecule.
54. The composition of embodiment 53, wherein the at least one lipid nanoparticle comprises:
about 47.5% to about 52.5% of ssPalmO-Ph-P4C2 by moles,
about 36.1% to about 41.1% of cholesterol by moles,
about 7.5% to about 12.5% of DOPE by moles, and
about 0.1% to about 3.9% of DMG-PEG2000 by moles.
55. The composition of embodiment 53 or embodiment 54, wherein the at least one lipid nanoparticle comprises:
about 49% to about 51% of ssPalmO-Ph-P4C2 by moles,
about 37.6% to about 39.6% of cholesterol by moles,
about 9% to about 11% of DOPE by moles, and
about 0.4% to about 2.4% of DMG-PEG2000 by moles.
56. The composition of any one of embodiments 53-55, wherein the at least one lipid nanoparticle comprises:
about 50% of ssPalmO-Ph-P4C2 by moles,
about 38.6% of cholesterol by moles,
about 10% of DOPE by moles, and
about 1.4% of DMG-PEG2000 by moles,
57. The composition of any one of embodiments 53-56, wherein the ratio of lipid to nucleic acid in the at least one lipid nanoparticle is about 195:1 to about 205:1 (w/w).
58. The composition of any one of embodiments 53-57, wherein the ratio of lipid to nucleic acid in the at least one lipid nanoparticle is about 200:1 (w/w).
59. The composition of any one of embodiments 53-58, wherein the at least one lipid nanoparticle comprises:
about 50% of ssPalmO-Ph-P4C2 by moles,
about 38.6% of cholesterol by moles,
about 10% of DOPE by moles, and
about 1.4% of DMG-PEG2000 by moles,
wherein the at least one lipid nanoparticle comprises at least one nucleic acid molecule, wherein the at least one nucleic acid molecule comprises at least one DNA molecule, and wherein the ratio of lipid to nucleic acid in the at least one lipid nanoparticle is about 200:1 (w/w).
60. A composition comprising at least one lipid nanoparticle comprising:
about 29.69% to about 39.69% of ssPalmO-Ph-P4C2 by moles,
about 34.74% to about 44.74% of cholesterol by moles,
about 19.69% to about 29.69% of DOPE by moles, and
about 0.01% to about 6.25% of DMG-PEG2000 by moles,
wherein the at least one lipid nanoparticle comprises at least one nucleic acid molecule, wherein the at least one nucleic acid molecule comprises at least one DNA molecule.
61. The composition of embodiment 60, wherein the at least one lipid nanoparticle comprises:
about 32.19% to about 37.19% of ssPalmO-Ph-P4C2 by moles,
about 37.24% to about 42.24% of cholesterol by moles,
about 22.19% to about 27.19% of DOPE by moles, and
about 0.1% to about 3.75% of DMG-PEG2000 by moles.
62. The composition of embodiment 60 or embodiment 61, wherein the at least one lipid nanoparticle comprises:
about 33.69% to about 35.69% of ssPalmO-Ph-P4C2 by moles,
about 38.74% to about 40.74% of cholesterol by moles,
about 23.69% to about 25.69% of DOPE by moles, and
about 0.25% to about 2.25% of DMG-PEG2000 by moles.
63. The composition of any one of embodiments 60-62, wherein the at least one lipid nanoparticle comprises:
about 34.69% of ssPalmO-Ph-P4C2 by moles,
about 39.74% of cholesterol by moles,
about 24.69% of DOPE by moles, and
about 1.25% of DMG-PEG2000 by moles.
64. The composition of any one of embodiments 60-63, wherein the ratio of lipid to nucleic acid in the at least one lipid nanoparticle is about 45:1 to about 55:1 (w/w).
65. The composition of any one of embodiments 60-64, wherein the ratio of lipid to nucleic acid in the at least one lipid nanoparticle is about 50:1 (w/w).
66. The composition of any one of embodiments 60-65, wherein the at least one lipid nanoparticle comprises:
about 34.69% of ssPalmO-Ph-P4C2 by moles,
about 39.74% of cholesterol by moles,
about 24.69% of DOPE by moles, and
about 1.25% of DMG-PEG2000 by moles,
wherein the at least one lipid nanoparticle comprises at least one nucleic acid molecule, wherein the at least one nucleic acid molecule comprises at least one DNA molecule, and wherein the ratio of lipid to nucleic acid in the at least one lipid nanoparticle is about 50:1 (w/w).
67. A composition comprising at least one lipid nanoparticle comprising:
about 45% to about 55% of ssPalmO-Ph-P4C2 by moles,
about 15% to about 25% of cholesterol by moles,
about 24.5% to about 34.5% of DOPE by moles, and
about 0.01% to about 5.5% of DMG-PEG2000 by moles,
wherein the at least one lipid nanoparticle comprises at least one nucleic acid molecule, wherein the at least one nucleic acid molecule comprises at least one DNA molecule.
68. The composition of embodiment 67, wherein the at least one lipid nanoparticle comprises:
about 47.5% to about 52.5% of ssPalmO-Ph-P4C2 by moles,
about 17.5% to about 22.5% of cholesterol by moles,
about 27% to about 32% of DOPE by moles, and
about 0.1% to about 3% of DMG-PEG2000 by moles.
69. The composition of embodiment 67 or embodiment 68, wherein the at least one lipid nanoparticle comprises:
about 49% to about 51% of ssPalmO-Ph-P4C2 by moles,
about 19% to about 21% of cholesterol by moles,
about 28.5% to about 30.5% of DOPE by moles, and
about 0.25% to about 1.5% of DMG-PEG2000 by moles.
70. The composition of any one of embodiments 67-69, wherein the at least one lipid nanoparticle comprises:
about 50% of ssPalmO-Ph-P4C2 by moles,
about 20% of cholesterol by moles,
about 29.5% of DOPE by moles, and
about 0.5% of DMG-PEG2000 by moles.
71. The composition of any one of embodiments 67-70, wherein the ratio of lipid to nucleic acid in the at least one lipid nanoparticle is about 95:1 to about 105:1 (w/w).
72. The composition of any one of embodiments 67-71, wherein the ratio of lipid to nucleic acid in the at least one lipid nanoparticle is about 100:1 (w/w).
73. The composition of any one of embodiments 67-71, wherein the at least one lipid nanoparticle comprises:
about 50% of ssPalmO-Ph-P4C2 by moles,
about 20% of cholesterol by moles,
about 29.5% of DOPE by moles, and
about 0.5% of DMG-PEG2000 by moles,
wherein the at least one lipid nanoparticle comprises at least one nucleic acid molecule, wherein the at least one nucleic acid molecule comprises at least one DNA molecule, and wherein the ratio of lipid to nucleic acid in the at least one nanoparticle is about 100:1 (w/w).
74. A composition comprising at least one lipid nanoparticle comprising:
about 49% to about 59% of ssPalmO-Ph-P4C2 by moles;
about 30% to about 40% of cholesterol by moles,
about 5% to about 15% of DOPE by moles, and
about 0.01% to about 6% of DMG-PEG2000 by moles.
wherein the at least one lipid nanoparticle comprises at least one nucleic acid molecule, wherein the at least one nucleic acid molecule comprises at least one RNA molecule.
75. The composition of embodiment 74, wherein the at least one lipid nanoparticle comprises:
about 51.5% to about 56.5% of ssPalmO-Ph-P4C2 by moles;
about 32.5% to about 37.5% of cholesterol by moles,
about 7.5% to about 12.5% of DOPE by moles, and
about 0.1% to about 3.5% of DMG-PEG2000 by moles.
76. The composition of embodiment 74 or embodiment 75, wherein the at least one lipid nanoparticle comprises:
about 53% to about 55% of ssPalmO-Ph-P4C2 by moles;
about 59% to about 61% of cholesterol by moles,
about 9% to about 11% of DOPE by moles, and
about 0.5% to about 2% of DMG-PEG2000 by moles.
77. The composition of any one of embodiments 74-76, wherein the at least one lipid nanoparticle comprises:
about 54% of ssPalmO-Ph-P4C2 by moles;
about 35% of cholesterol by moles,
about 10% of DOPE by moles, and
about 1% of DMG-PEG2000 by moles.
78. The composition of any one of embodiments 74-77, wherein the ratio of lipid to nucleic acid in that least one lipid nanoparticle is about 95:1 to about 105:1 (w/w).
79. The composition of any one of embodiments 74-78, wherein the ratio of lipid to nucleic acid in the at least one nanoparticle is about 100:1 (w/w).
80. The composition of any one of embodiments 74-79, wherein the at least one lipid nanoparticle comprises:
about 54% of ssPalmO-Ph-P4C2 by moles;
about 35% of cholesterol by moles,
about 10% of DOPE by moles, and
about 1% of DMG-PEG2000 by moles.
   wherein the at least one lipid nanoparticle comprises at least one nucleic acid molecule, wherein the at least one nucleic acid molecule comprises at least one RNA molecule, and wherein the ratio of lipid to nucleic acid in the at least one lipid nanoparticle is about 100:1 (w/w).
81. A composition comprising at least one lipid nanoparticle comprising:
about 22.84% to about 32.84% of ssPalmO-Ph-P4C2 by moles;
about 51.25% to about 61.25% of cholesterol by moles,
about 8.46% to about 18.46% of DOPE by moles, and
about 0.01% to about 7.45% of DMG-PEG2000 by moles.
wherein the at least one lipid nanoparticle comprises at least one nucleic acid molecule, wherein the at least one nucleic acid molecule comprises at least one RNA molecule.
82. The composition of embodiment 81, wherein the at least one lipid nanoparticle comprises:
about 25.34% to about 30.34% of ssPalmO-Ph-P4C2 by moles;
about 53.75% to about 58.75% of cholesterol by moles,
about 10.96% to about 15.96% of DOPE by moles, and
about 0.1% to about 4.95% of DMG-PEG2000 by moles.
83. The composition of embodiment 81 or embodiment 82, wherein the at least one lipid nanoparticle comprises:
about 26.84% to about 28.84% of ssPalmO-Ph-P4C2 by moles;
about 55.25% to about 57.25% of cholesterol by moles,
about 12.46% to about 13.46% of DOPE by moles, and
about 1.45% to about 3.45% of DMG-PEG2000 by moles.
84. The composition of any one of embodiments 81-83, wherein the at least one lipid nanoparticle comprises:
about 27.84% of ssPalmO-Ph-P4C2 by moles;
about 56.25% of cholesterol by moles,
about 13.46% of DOPE by moles, and
about 2.45% of DMG-PEG2000 by moles.
85. The composition of any one of embodiments 81-84, wherein the ratio of lipid to nucleic acid in that least one lipid nanoparticle is about 83:1 to about 93:1 (w/w).
86. The composition of any one of embodiments 81-85, wherein the ratio of lipid to nucleic acid in the at least one nanoparticle is about 88:1 (w/w).
87. The composition of any one of embodiments 81-86, wherein the at least one lipid nanoparticle comprises:
about 27.84% of ssPalmO-Ph-P4C2 by moles;
about 56.25% of cholesterol by moles,
about 13.46% of DOPE by moles, and
about 2.45% of DMG-PEG2000 by moles.
   wherein the at least one lipid nanoparticle comprises at least one nucleic acid molecule, wherein the at least one nucleic acid molecule comprises at least one RNA molecule, and wherein the ratio of lipid to nucleic acid in the at least one lipid nanoparticle is about 88:1 (w/w).
88. A composition comprising at least one lipid nanoparticle comprising:
about 22.9% to about 32.9% of ssPalmO-Ph-P4C2 by moles;
about 46.51% to about % of cholesterol by moles,
about 13.59% to about 23.59% of DOPE by moles, and
about 0.01% to about 7% of DMG-PEG2000 by moles.
wherein the at least one lipid nanoparticle comprises at least one nucleic acid molecule, wherein the at least one nucleic acid molecule comprises at least one RNA molecule.
89. The composition of embodiment 88, wherein the at least one lipid nanoparticle comprises:
about 25.4% to about 30.4% of ssPalmO-Ph-P4C2 by moles;
about 49.01% to about % of cholesterol by moles,
about 16.09% to about 21.09% of DOPE by moles, and
about 0.1% to about 4.5% of DMG-PEG2000 by moles.
90. The composition of embodiment 88 or embodiment 89, wherein the at least one lipid nanoparticle comprises:
about 26.9% to about 28.9% of ssPalmO-Ph-P4C2 by moles;
about 50.51% to about 52.51% of cholesterol by moles,
about 17.59% to about 19.59% of DOPE by moles, and
about 1% to about 3% of DMG-PEG2000 by moles.
91. The composition of any one of embodiments 88-90, wherein the at least one lipid nanoparticle comprises:
about 27.9% of ssPalmO-Ph-P4C2 by moles;
about 51.51% of cholesterol by moles,
about 18.59% of DOPE by moles, and
about 2% of DMG-PEG2000 by moles.
92. The composition of any one of embodiments 88-91, wherein the ratio of lipid to nucleic acid in that least one lipid nanoparticle is about 95:1 to about 105:1 (w/w).
93. The composition of any one of embodiments 88-92, wherein the ratio of lipid to nucleic acid in the at least one nanoparticle is about 100:1 (w/w).
94. The composition of any one of embodiments 88-93, wherein the at least one lipid nanoparticle comprises:
about 27.9% of ssPalmO-Ph-P4C2 by moles;
about 51.51% of cholesterol by moles,
about 18.59% of DOPE by moles, and
about 2% of DMG-PEG2000 by moles.
   wherein the at least one lipid nanoparticle comprises at least one nucleic acid molecule, wherein the at least one nucleic acid molecule comprises at least one RNA molecule, and wherein the ratio of lipid to nucleic acid in the at least one lipid nanoparticle is about 100:1 (w/w).
95. A composition comprising at least one lipid nanoparticle comprising:
about 55% to about 65% of ssPalmO-Ph-P4C2 by moles;
about 21.4% to about 31.4% of cholesterol by moles,
about 6.6% to about 16.6% of DOPE by moles, and
about 0.01% to about 7% of DMG-PEG2000 by moles.
wherein the at least one lipid nanoparticle comprises at least one nucleic acid molecule, wherein the at least one nucleic acid molecule comprises at least one RNA molecule.
96. The composition of embodiment 95, wherein the at least one lipid nanoparticle comprises:
about 57.5% to about 62.5% of ssPalmO-Ph-P4C2 by moles;
about 23.9% to about 28.9% of cholesterol by moles,
about 9.1% to about 14.1% of DOPE by moles, and
about 0.1% to about 4.5% of DMG-PEG2000 by moles.
97. The composition of embodiment 95 or embodiment 96, wherein the at least one lipid nanoparticle comprises:
about 59% to about 61% of ssPalmO-Ph-P4C2 by moles;
about 25.4% to about 27.4% of cholesterol by moles,
about 10.6% to about 12.6% of DOPE by moles, and
about 1% to about 3% of DMG-PEG2000 by moles.
98. The composition of any one of embodiments 95-97, wherein the at least one lipid nanoparticle comprises:
about 60% of ssPalmO-Ph-P4C2 by moles;
about 26.4% of cholesterol by moles,
about 11.6% of DOPE by moles, and
about 2% of DMG-PEG2000 by moles.
99. The composition of any one of embodiments 95-98, wherein the ratio of lipid to nucleic acid in that least one lipid nanoparticle is about 65:1 to about 75:1 (w/w).
100. The composition of any one of embodiments 95-99, wherein the ratio of lipid to nucleic acid in the at least one nanoparticle is about 70:1 (w/w).
101. The composition of any one of embodiments 95-100, wherein the at least one lipid nanoparticle comprises:
about 60% of ssPalmO-Ph-P4C2 by moles;
about 26.4% of cholesterol by moles,
about 11.6% of DOPE by moles, and
about 2% of DMG-PEG2000 by moles.
   wherein the at least one lipid nanoparticle comprises at least one nucleic acid molecule, wherein the at least one nucleic acid molecule comprises at least one RNA molecule, and wherein the ratio of lipid to nucleic acid in the at least one lipid nanoparticle is about 70:1 (w/w).
102. A composition comprising at least one lipid nanoparticle comprising:
about 25.37% to about 35.37% of ssPalmO-Ph-P4C2 by moles;
about 32.27% to about 42.27% of cholesterol by moles,
about 25.36% to about 35.36% of DOPE by moles, and
about 0.01% to about 7% of DMG-PEG2000 by moles.
wherein the at least one lipid nanoparticle comprises at least one nucleic acid molecule, wherein the at least one nucleic acid molecule comprises at least one RNA molecule.
103. The composition of embodiment 102, wherein the at least one lipid nanoparticle comprises:
about 27.87% to about 32.87% of ssPalmO-Ph-P4C2 by moles;
about 34.77% to about 39.77% of cholesterol by moles,
about 27.86% to about 32.86% of DOPE by moles, and
about 0.1% to about 4.5% of DMG-PEG2000 by moles.
104. The composition of embodiment 102 or embodiment 103, wherein the at least one lipid nanoparticle comprises:
about 29.37% to about 31.37% of ssPalmO-Ph-P4C2 by moles;
about 36.27% to about 38.27% of cholesterol by moles,
about 29.36% to about 31.36% of DOPE by moles, and
about 1% to about 3% of DMG-PEG2000 by moles.
105. The composition of any one of embodiments 102-104, wherein the at least one lipid nanoparticle comprises:
about 30.37% of ssPalmO-Ph-P4C2 by moles;
about 37.27% of cholesterol by moles,
about 30.36% of DOPE by moles, and
about 2% of DMG-PEG2000 by moles.
106. The composition of any one of embodiments 102-105, wherein the ratio of lipid to nucleic acid in that least one lipid nanoparticle is about 95:1 to about 105:1 (w/w).
107. The composition of any one of embodiments 102-106, wherein the ratio of lipid to nucleic acid in the at least one nanoparticle is about 100:1 (w/w).
108. The composition of any one of embodiments 102-107, wherein the at least one lipid nanoparticle comprises:
about 30.37% of ssPalmO-Ph-P4C2 by moles;
about 37.27% of cholesterol by moles,
about 30.36% of DOPE by moles, and
about 2% of DMG-PEG2000 by moles.
   wherein the at least one lipid nanoparticle comprises at least one nucleic acid molecule, wherein the at least one nucleic acid molecule comprises at least one RNA molecule, and wherein the ratio of lipid to nucleic acid in the at least one lipid nanoparticle is about 100:1 (w/w).
109. A composition comprising at least one lipid nanoparticle comprising:
about 45% to about 55% of ssPalmO-Ph-P4C2 by moles,
about 15% to about 25% of cholesterol by moles,
about 23.7% to about 33.7% of DOPE by moles, and
about 0.01% to about 6.3% of DMG-PEG2000 by moles,
wherein the at least one lipid nanoparticle comprises at least one nucleic acid molecule, wherein the at least one nucleic acid molecule comprises at least one DNA molecule.
110. The composition of embodiment 109, wherein the at least one lipid nanoparticle comprises:
about 47.5% to about 52.5% of ssPalmO-Ph-P4C2 by moles,
about 17.5% to about 22.5% of cholesterol by moles,
about 26.2% to about 31.2% of DOPE by moles, and
about 0.1% to about 4% of DMG-PEG2000 by moles.
111. The composition of embodiment 109 or embodiment 110, wherein the at least one lipid nanoparticle comprises:
about 49% to about 51% of ssPalmO-Ph-P4C2 by moles,
about 19% to about 21% of cholesterol by moles,
about 27.7% to about 29.7% of DOPE by moles, and
about 0.25% to about 2.3% of DMG-PEG2000 by moles.
112. The composition of any one of embodiments 109-111, wherein the at least one lipid nanoparticle comprises:
about 50% of ssPalmO-Ph-P4C2 by moles,
about 20% of cholesterol by moles,
about 28.7% of DOPE by moles, and
about 1.3% of DMG-PEG2000 by moles.
113. The composition of any one of embodiments 109-112, wherein the ratio of lipid to nucleic acid in the at least one lipid nanoparticle is about 45:1 to about 55:1 (w/w).
114. The composition of any one of embodiments 109-113, wherein the ratio of lipid to nucleic acid in the at least one lipid nanoparticle is about 50:1 (w/w).
115. The composition of any one of embodiments 109-114, wherein the at least one lipid nanoparticle comprises:
about 50% of ssPalmO-Ph-P4C2 by moles,
about 20% of cholesterol by moles,
about 28.7% of DOPE by moles, and
about 1.3% of DMG-PEG2000 by moles,
wherein the at least one lipid nanoparticle comprises at least one nucleic acid molecule, wherein the at least one nucleic acid molecule comprises at least one DNA molecule, and wherein the ratio of lipid to nucleic acid in the at least one nanoparticle is about 50:1 (w/w).
116. A composition comprising at least one lipid nanoparticle comprising:
about 49% to about 59% of ssPalmO-Ph-P4C2 by moles,
about 30% to about 40% of cholesterol by moles,
about 5% to about 15% of phospholipid by moles, and
about 0.01% to about 6% of DMG-PEG2000 by moles,
wherein the at least one lipid nanoparticle comprises at least one nucleic acid molecule.
117. The composition of embodiment 116, wherein the at least one lipid nanoparticle comprises:
about 51.5% to about 56.5% of ssPalmO-Ph-P4C2 by moles,
about 32.5% to about 37.5% of cholesterol by moles,
about 7.5% to about 12.5% of phospholipid by moles, and
about 0.1% to about 3.5% of DMG-PEG2000 by moles.
118. The composition of embodiment 116 or embodiment 117, wherein the at least one lipid nanoparticle comprises:
about 53% to about 55% of ssPalmO-Ph-P4C2 by moles,
about 34% to about 36% of cholesterol by moles,
about 9% to about 11% of phospholipid by moles, and
about 0.25% to about 2% of DMG-PEG2000 by moles.
119. The composition of any one of embodiments 116-118, wherein the at least one lipid nanoparticle comprises:
about 54% of ssPalmO-Ph-P4C2 by moles,
about 35% of cholesterol by moles,
about 10% of phospholipid by moles, and
about 1% of DMG-PEG2000 by moles.
120. A composition comprising at least one lipid nanoparticle comprising:
about 54% to about 64% of ssPalmO-Ph-P4C2 by moles,
about 30% to about 40% of cholesterol by moles,
about 0.1% to about 10% of phospholipid by moles, and
about 0.01% to about 6% of DMG-PEG2000 by moles,
wherein the at least one lipid nanoparticle comprises at least one nucleic acid molecule.
121. The composition of embodiment 120, wherein the at least one lipid nanoparticle comprises:
about 56.5% to about 61.5% of ssPalmO-Ph-P4C2 by moles,
about 32.5% to about 37.5% of cholesterol by moles,
about 2.5% to about 7.5% of phospholipid by moles, and
about 0.1% to about 3.5% of DMG-PEG2000 by moles.
122. The composition of embodiment 120 or embodiment 121, wherein the at least one lipid nanoparticle comprises:
about 58% to about 60% of ssPalmO-Ph-P4C2 by moles,
about 34% to about 36% of cholesterol by moles,
about 4% to about 6% of phospholipid by moles, and
about 0.25% to about 2% of DMG-PEG2000 by moles.
123. The composition of any one of embodiments 120-122, wherein the at least one lipid nanoparticle comprises:
about 59% of ssPalmO-Ph-P4C2 by moles,
about 35% of cholesterol by moles,
about 5% of phospholipid by moles, and
about 1% of DMG-PEG2000 by moles.
124. A composition comprising at least one lipid nanoparticle comprising:
about 49% to about 59% of ssPalmO-Ph-P4C2 by moles,
about 35% to about 45% of cholesterol by moles,
about 0.1% to about 10% of phospholipid by moles, and
about 0.01% to about 6% of DMG-PEG2000 by moles,
wherein the at least one lipid nanoparticle comprises at least one nucleic acid molecule.
125. The composition of embodiment 124, wherein the at least one lipid nanoparticle comprises:
about 51.5% to about 56.5% of ssPalmO-Ph-P4C2 by moles,
about 37.5% to about 42.5% of cholesterol by moles,
about 2.5% to about 7.5% of phospholipid by moles, and
about 0.1% to about 3.5% of DMG-PEG2000 by moles.
126. The composition of embodiment 124 or embodiment 125, wherein the at least one lipid nanoparticle comprises:
about 53% to about 55% of ssPalmO-Ph-P4C2 by moles,
about 39% to about 41% of cholesterol by moles,
about 4% to about 6% of phospholipid by moles, and
about 0.25% to about 2% of DMG-PEG2000 by moles.
127. The composition of any one of embodiments 124-126, wherein the at least one lipid nanoparticle comprises:
about 54% of ssPalmO-Ph-P4C2 by moles,
about 40% of cholesterol by moles,
about 5% of phospholipid by moles, and
about 1% of DMG-PEG2000 by moles.
128. A composition comprising at least one lipid nanoparticle comprising:
about 51.5% to about 61.5% of ssPalmO-Ph-P4C2 by moles,
about 32.5% to about 42.5% of cholesterol by moles,
about 0.1% to about 10% of phospholipid by moles, and
about 0.01% to about 6% of DMG-PEG2000 by moles,
wherein the at least one lipid nanoparticle comprises at least one nucleic acid molecule.
129. The composition of embodiment 128, wherein the at least one lipid nanoparticle comprises:
about 54% to about 59% of ssPalmO-Ph-P4C2 by moles,
about 35% to about 40% of cholesterol by moles,
about 2.5% to about 7.5% of phospholipid by moles, and
about 0.1% to about 3.5% of DMG-PEG2000 by moles.
130. The composition of embodiment 128 or embodiment 129, wherein the at least one lipid nanoparticle comprises:
about 55.5% to about 57.5% of ssPalmO-Ph-P4C2 by moles,
about 36.5% to about 38.5% of cholesterol by moles,
about 4% to about 6% of phospholipid by moles, and
about 0.25% to about 2% of DMG-PEG2000 by moles.
131. The composition of any one of embodiments 128-130, wherein the at least one lipid nanoparticle comprises:
about 56.5% of ssPalmO-Ph-P4C2 by moles,
about 37.5% of cholesterol by moles,
about 5% of phospholipid by moles, and
about 1% of DMG-PEG2000 by moles.
132. A composition comprising at least one lipid nanoparticle comprising:
about 44.4% to about 54.4% of ssPalmO-Ph-P4C2 by moles,
about 39% to about 49% of cholesterol by moles,
about 0.1% to about 10% of phospholipid by moles, and
about 0.01% to about 6.6% of DMG-PEG2000 by moles,
wherein the at least one lipid nanoparticle comprises at least one nucleic acid molecule.
133. The composition of embodiment 132, wherein the at least one lipid nanoparticle comprises:
about 46.9% to about 51.9% of ssPalmO-Ph-P4C2 by moles,
about 41.5% to about 46.5% of cholesterol by moles,
about 2.5% to about 7.5% of phospholipid by moles, and
about 0.1% to about 4.1% of DMG-PEG2000 by moles.
134. The composition of embodiment 132 or embodiment 133, wherein the at least one lipid nanoparticle comprises:
about 48.4% to about 50.4% of ssPalmO-Ph-P4C2 by moles,
about 43% to about 45% of cholesterol by moles,
about 4% to about 6% of phospholipid by moles, and
about 0.6% to about 2.6% of DMG-PEG2000 by moles.
135. The composition of any one of embodiments 132-134, wherein the at least one lipid nanoparticle comprises:
about 49.4% of ssPalmO-Ph-P4C2 by moles,
about 44% of cholesterol by moles,
about 5% of phospholipid by moles, and
about 1.6% of DMG-PEG2000 by moles.
136. A composition comprising at least one lipid nanoparticle comprising:
about 55% to about 65% of ssPalmO-Ph-P4C2 by moles,
about 27.8% to about 37.8% of cholesterol by moles,
about 0.1% to about 10% of phospholipid by moles, and
about 0.01% to about 7.2% of DMG-PEG2000 by moles,
wherein the at least one lipid nanoparticle comprises at least one nucleic acid molecule.
137. The composition of embodiment 136, wherein the at least one lipid nanoparticle comprises:
about 57.5% to about 62.5% of ssPalmO-Ph-P4C2 by moles,
about 30.3% to about 35.3% of cholesterol by moles,
about 2.5% to about 7.5% of phospholipid by moles, and
about 0.1% to about 4.7% of DMG-PEG2000 by moles.
138. The composition of embodiment 136 or embodiment 137, wherein the at least one lipid nanoparticle comprises:
about 59% to about 61% of ssPalmO-Ph-P4C2 by moles,
about 31.8% to about 33.8% of cholesterol by moles,
about 4% to about 6% of phospholipid by moles, and
about 1.2% to about 2.2% of DMG-PEG2000 by moles.
139. The composition of any one of embodiments 136-138, wherein the at least one lipid nanoparticle comprises:
about 60% of ssPalmO-Ph-P4C2 by moles,
about 32.8% of cholesterol by moles,
about 5% of phospholipid by moles, and
about 2.2% of DMG-PEG2000 by moles.
140. A composition comprising at least one lipid nanoparticle comprising:
about 55% to about 65% of ssPalmO-Ph-P4C2 by moles,
about 29% to about 39% of cholesterol by moles,
about 0.1% to about 10% of phospholipid by moles, and
about 0.01% to about 6% of DMG-PEG2000 by moles,
wherein the at least one lipid nanoparticle comprises at least one nucleic acid molecule.
141. The composition of embodiment 140, wherein the at least one lipid nanoparticle comprises:
about 57.5% to about 62.5% of ssPalmO-Ph-P4C2 by moles,
about 31.5% to about 36.5% of cholesterol by moles,
about 2.5% to about 7.5% of phospholipid by moles, and
about 0.1% to about 3.5% of DMG-PEG2000 by moles.
142. The composition of embodiment 140 or embodiment 141, wherein the at least one lipid nanoparticle comprises:
about 59% to about 61% of ssPalmO-Ph-P4C2 by moles,
about 33% to about 35% of cholesterol by moles,
about 4% to about 6% of phospholipid by moles, and
about 0.25% to about 2% of DMG-PEG2000 by moles.
143. The composition of any one of embodiments 140-142, wherein the at least one lipid nanoparticle comprises:
about 60% of ssPalmO-Ph-P4C2 by moles,
about 34% of cholesterol by moles,
about 5% of phospholipid by moles, and
about 1% of DMG-PEG2000 by moles.
144. A composition comprising at least one lipid nanoparticle comprising:
about 36.8% to about 46.8% of ssPalmO-Ph-P4C2 by moles,
about 47.2% to about 57.2% of cholesterol by moles,
about 0.1% to about 10% of phospholipid by moles, and
about 0.01% to about 6% of DMG-PEG2000 by moles,
wherein the at least one lipid nanoparticle comprises at least one nucleic acid molecule.
145. The composition of embodiment 144, wherein the at least one lipid nanoparticle comprises:
about 39.3% to about 44.3% of ssPalmO-Ph-P4C2 by moles,
about 49.7% to about 54.7% of cholesterol by moles,
about 2.5% to about 7.5% of phospholipid by moles, and
about 0.1% to about 3.5% of DMG-PEG2000 by moles.
146. The composition of embodiment 144 or embodiment 145, wherein the at least one lipid nanoparticle comprises:
about 40.8% to about 42.8% of ssPalmO-Ph-P4C2 by moles,
about 51.2% to about 53.2% of cholesterol by moles,
about 4% to about 6% of phospholipid by moles, and
about 0.25% to about 2% of DMG-PEG2000 by moles.
147. The composition of any one of embodiments 144-146, wherein the at least one lipid nanoparticle comprises:
about 41.8% of ssPalmO-Ph-P4C2 by moles,
about 52.2% of cholesterol by moles,
about 5% of phospholipid by moles, and
about 1% of DMG-PEG2000 by moles.
148. The composition of any one of embodiments 116-147, wherein the at least one nucleic acid molecule is an RNA molecule.
149. The composition of embodiment 148, wherein the RNA molecule is an mRNA molecule.
150. The composition of embodiment 149, wherein the mRNA molecule further comprises a 5′-CAP.
151. The composition of any one of embodiments 116-147, wherein the at least one nucleic acid molecule is a DNA molecule.
152. The composition of embodiment 151, wherein the DNA molecule is a DoggyBone DNA molecule.
153. The composition of embodiment 151, wherein the DNA molecule is a DNA nanoplasmid.
154. The composition of any one of embodiments 116-153, wherein the phospholipid is DOPE.
155. The composition of any one of embodiments 116-153, wherein the phospholipid is DOPC.
156. The composition of any one of embodiments 116-153, wherein the phospholipid is DSPC.
157. The composition of any one of embodiments 116-156, wherein the ratio of lipid to nucleic acid in the at least one lipid nanoparticle is about 55:1 to about 110:1 (w/w).
158. The composition of any one of embodiments 116-156, wherein the ratio of lipid to nucleic acid in the at least one lipid nanoparticle is about 20:1 (w/w).
159. The composition of any one of embodiments 116-156, wherein the ratio of lipid to nucleic acid in the at least one lipid nanoparticle is about 40:1 (w/w).
160. The composition of any one of embodiments 116-156, wherein the ratio of lipid to nucleic acid in the at least one lipid nanoparticle is about 60:1 (w/w).
161. The composition of any one of embodiments 116-156, wherein the ratio of lipid to nucleic acid in the at least one lipid nanoparticle is about 75:1 (w/w).
162. The composition of any one of embodiments 116-156, wherein the ratio of lipid to nucleic acid in the at least one lipid nanoparticle is about 80:1 (w/w).
163. The composition of any one of embodiments 116-156, wherein the ratio of lipid to nucleic acid in the at least one lipid nanoparticle is about 100:1 (w/w).
164. The composition of any one of the preceding embodiments, wherein the at least one lipid nanoparticle comprises:
about 54% of ssPalmO-Ph-P4C2 by moles,
about 35% of cholesterol by moles,
about 10% of DOPE by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).
165. The composition of any one of the preceding embodiments, wherein the at least one lipid nanoparticle comprises:
about 54% of ssPalmO-Ph-P4C2 by moles,
about 35% of cholesterol by moles,
about 10% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).
166. The composition of any one of the preceding embodiments, wherein the at least one lipid nanoparticle comprises:
about 54% of ssPalmO-Ph-P4C2 by moles,
about 35% of cholesterol by moles,
about 10% of DSPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).
167. The composition of any one of the preceding embodiments, wherein the at least one lipid nanoparticle comprises:
about 59% of ssPalmO-Ph-P4C2 by moles,
about 35% of cholesterol by moles,
about 5% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).
168. The composition of any one of the preceding embodiments, wherein the at least one lipid nanoparticle comprises:
about 59% of ssPalmO-Ph-P4C2 by moles,
about 35% of cholesterol by moles,
about 5% of DSPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).
169. The composition of any one of the preceding embodiments, wherein the at least one lipid nanoparticle comprises:
about 54% of ssPalmO-Ph-P4C2 by moles,
about 40% of cholesterol by moles,
about 5% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).
170. The composition of any one of the preceding embodiments, wherein the at least one lipid nanoparticle comprises:
about 54% of ssPalmO-Ph-P4C2 by moles,
about 40% of cholesterol by moles,
about 5% of DSPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).
171. The composition of any one of the preceding embodiments, wherein the at least one lipid nanoparticle comprises:
about 56.5% of ssPalmO-Ph-P4C2 by moles,
about 37.5% of cholesterol by moles,
about 5% of DSPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).
172. The composition of any one of the preceding embodiments, wherein the at least one lipid nanoparticle comprises:
about 56.5% of ssPalmO-Ph-P4C2 by moles,
about 37.5% of cholesterol by moles,
about 5% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).
173. The composition of any one of the preceding embodiments, wherein the at least one lipid nanoparticle comprises:
about 54% of ssPalmO-Ph-P4C2 by moles,
about 35% of cholesterol by moles,
about 10% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 75:1 (w/w).
174. The composition of any one of the preceding embodiments, wherein the at least one lipid nanoparticle comprises:
about 54% of ssPalmO-Ph-P4C2 by moles,
about 35% of cholesterol by moles,
about 10% of DOPE by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 75:1 (w/w).
175. The composition of any one of the preceding embodiments, wherein the at least one lipid nanoparticle comprises:
about 59% of ssPalmO-Ph-P4C2 by moles,
about 35% of cholesterol by moles,
about 5% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 75:1 (w/w).
176. The composition of any one of the preceding embodiments, wherein the at least one lipid nanoparticle comprises:
about 54% of ssPalmO-Ph-P4C2 by moles,
about 35% of cholesterol by moles,
about 10% of DOPE by moles, and
about 1% of DMG-PEG2000 by moles,
wherein the at least one lipid nanoparticle comprises at least one nucleic acid molecule, wherein the at least one nucleic acid molecule comprises at least one DNA molecule, and wherein the ratio of lipid to nucleic acid in the at least one nanoparticle is about 100:1 (w/w).
177. The composition of any one of the preceding embodiments, wherein the at least one lipid nanoparticle comprises:
about 54% of ssPalmO-Ph-P4C2 by moles,
about 35% of cholesterol by moles,
about 10% of DOPE by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 60:1 (w/w).
178. The composition of any one of the preceding embodiments, wherein the at least one lipid nanoparticle comprises:
about 54% of ssPalmO-Ph-P4C2 by moles,
about 35% of cholesterol by moles,
about 10% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 60:1 (w/w).
179. The composition of any one of the preceding embodiments, wherein the at least one lipid nanoparticle comprises:
about 49.4% of ssPalmO-Ph-P4C2 by moles,
about 44% of cholesterol by moles,
about 5% of DOPC by moles, and
about 1.6% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 60:1 (w/w).
180. The composition of any one of the preceding embodiments, wherein the at least one lipid nanoparticle comprises:
about 60% of ssPalmO-Ph-P4C2 by moles,
about 32.8% of cholesterol by moles,
about 5% of DSPC by moles, and
about 2.2% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 60:1 (w/w).
181. The composition of any one of the preceding embodiments, wherein the at least one lipid nanoparticle comprises:
about 60% of ssPalmO-Ph-P4C2 by moles,
about 34% of cholesterol by moles,
about 5% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 40:1 (w/w).
182. The composition of any one of the preceding embodiments, wherein the at least one lipid nanoparticle comprises:
about 41.8% of ssPalmO-Ph-P4C2 by moles,
about 52.2% of cholesterol by moles,
about 5% of DSPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 60:1 (w/w).
183. The composition of any one of the preceding embodiments, wherein the at least one lipid nanoparticle comprises:
about 54% of ssPalmO-Ph-P4C2 by moles,
about 35% of cholesterol by moles,
about 10% of DSPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 60:1 (w/w).

184. The composition of any one of the preceding embodiments, wherein the mRNA comprises cytidine residues that are 5-methylcytidine (5-MeC).
185. A composition comprising at least one lipid nanoparticle comprising:
about 30% to about 40% of C12-200 by moles;
about 36.84% to about 46.84% of cholesterol by moles,
about 15% to about 25% of DOPE by moles, and
about 0.01% to about 8.16% of DMG-PEG2000 by moles,
wherein the at least one lipid nanoparticle comprises at least one nucleic acid molecule.
186. The composition of embodiment 185, wherein the at least one lipid nanoparticle comprises:
about 32.5% to about 37.5% of C12-200 by moles;
about 39.34% to about 44.34% of cholesterol by moles,
about 17.5% to about 22.5% of DOPE by moles, and
about 0.1% to about 5.66% of DMG-PEG2000 by moles.
187. The composition of embodiment 185 or embodiment 186, wherein the at least one lipid nanoparticle comprises:
about 34% to about 36% of C12-200 by moles;
about 40.84% to about 42.84% of cholesterol by moles,
about 19% to about 21% of DOPE by moles, and
about 2.16% to about 4.16% of DMG-PEG2000 by moles.
188. The composition of any one of embodiments 185-187, wherein the at least one lipid nanoparticle comprises:
about 35% of C12-200 by moles;
about 41.84% of cholesterol by moles,
about 20% of DOPE by moles, and
about 3.16% of DMG-PEG2000 by moles.
189. The composition of any one of embodiments 185-188, wherein the at least one nucleic acid molecule comprises at least one DNA molecule.
190. The composition of embodiment 189, wherein the DNA molecule is a DoggyBone DNA molecule.
191. The composition of embodiment 190, wherein the DNA molecule is a DNA nanoplasmid.
192. The composition of any one of embodiments 185-191, wherein the ratio of lipid to nucleic acid in the at least one lipid nanoparticle is about 70:1 to about 90:1 (w/w).
193. The composition of any one of embodiments 185-191, wherein the ratio of lipid to nucleic acid in the at least one lipid nanoparticle is about 20:1 (w/w).
194. The composition of any one of embodiments 185-191, wherein the ratio of lipid to nucleic acid in the at least one lipid nanoparticle is about 40:1 (w/w).
195. The composition of any one of embodiments 185-191, wherein the ratio of lipid to nucleic acid in the at least one lipid nanoparticle is about 60:1 (w/w).
196. The composition of any one of embodiments 185-191, wherein the ratio of lipid to nucleic acid in the at least one lipid nanoparticle is about 75:1 (w/w).
197. The composition of any one of embodiments 185-191, wherein the ratio of lipid to nucleic acid in the at least one lipid nanoparticle is about 80:1 (w/w).
198. The composition of any one of embodiments 185-191, wherein the ratio of lipid to nucleic acid in the at least one lipid nanoparticle is about 100:1 (w/w).

Pharmaceutical Compositions of the Present Disclosure

In some aspects, the present disclosure provides a pharmaceutical composition comprising at least one lipid nanoparticle of the present disclosure.

In some aspects, the present disclosure provides a pharmaceutical composition comprising at least one lipid nanoparticle of the present disclosure. In some aspects, the present disclosure provides a pharmaceutical composition comprising at least one first nanoparticle of the present disclosure and at least one second nanoparticle of the present disclosure, wherein the at least one first nanoparticle comprises at least one nucleic acid molecule encoding at least one transposase, wherein the at least one second nanoparticle comprises at least one nucleic acid molecule encoding at least one transposon.

In some aspects, the present disclosure provides a composition comprising at least one cell that has been contacted by at least one nanoparticle of the present disclosure. In some aspects, the present disclosure provides a composition comprising at least one cell that has been genetically modified using at least one nanoparticle of the present disclosure. In some aspects, the present disclosure provides a composition comprising at least one cell that has been genetically modified using any method of the present disclosure.

Methods of the Present Disclosure

The present disclosure provides a method of delivering at least one nucleic acid to at least one cell comprising contacting the at least one cell with at least one composition of the present disclosure. The present disclosure provides a method of delivering at least one nucleic acid to at least one cell comprising contacting the at least one cell with at least one nanoparticle of the present disclosure.

In all methods, compositions and kits of the present disclosure, an at least one cell can be a liver cell. A liver cell can include, but is not limited to, a hepatocyte, a hepatic stellate cell, Kupffer cell or a liver sinusoidal endothelial cell.

In some aspects of any methods of the present disclosure, a cell can be in vivo, ex vivo or in vitro. In some aspects, any of the methods of the present disclosure can be applied in vivo, ex vivo or in vitro.

The present disclosure provides a method of genetically modifying at least one cell comprising contacting the at least one cell with at least one composition of the present disclosure. The present disclosure provides a method of genetically modifying at least one cell comprising contacting the at least one cell with at least one lipid nanoparticle of the present disclosure.

In some aspects, genetically modifying a cell can comprise delivering at least one exogenous nucleic acid to the cell such that the cell expresses at least one protein that the cell otherwise would not normally express, or such that the at least one cell expresses at least one protein at a level that is higher than the level that the cell would otherwise normally express the at least one protein, or such that the cell expresses at least one protein at a level that is lower than the level that the cell would otherwise normally express. In some aspects, genetically modifying a cell can comprise delivering at least one exogenous nucleic to the cell such that at least one exogenous nucleic acid is integrated into the genome of the at least one cell.

In some aspects, contacting an at least one cell with at least one lipid nanoparticle of the present disclosure can result in the at least one cell expressing at least one exogenous protein at a level that is at least about a 2 fold, or at least about a 3 fold, or at least about a 4 fold, or at least about a 5 fold, or at least about a 6 fold, or at least about a 7 fold, or at least about an 8 fold, or at least about a 9 fold, or at least about a 10 fold, or at last about a 15 fold, or at least about a 20 fold, or at least about a 25 fold, or at least about a 30 fold, or at least about a 50 fold increase as compared to the expression level of the exogenous protein induced by contacting the at least one cell with at least one control lipid nanoparticle.

In some aspects, the methods of the present disclosure can yield a plurality of cells, wherein at least about 1%, or at least about 2%, or at least about 3%, or at least about 4%, or at least about 5%, or at least 10%, or at least 15%, or at least 20%, or at least 25%, or at least 30%, or at least 35%, or at least 40%, or at least 45%, or at least 50%, or at least 55%, or at least 60%, or at least about 65%, or at least about 70%, or at least about 75%, or at least about 80%, or at least about 85%, or at least about 90%, or at least about 95%, or at least about 99% of the cell in the plurality express at least one protein that was encoded in at least one nucleic acid that was delivered to the plurality of cells via a nanoparticle of the present disclosure.

In some aspects, the methods of the present disclosure can yield a plurality of cells, wherein at least about 1%, or at least about 2%, or at least about 3%, or at least about 4%, or at least about 5%, or at least 10%, or at least 15%, or at least 20%, or at least 25%, or at least 30%, or at least 35%, or at least 40%, or at least 45%, or at least 50%, or at least 55%, or at least 60%, or at least about 65%, or at least about 70%, or at least about 75%, or at least about 80%, or at least about 85%, or at least about 90%, or at least about 95%, or at least about 99% of the cells in the plurality are hepatocytes, a hepatic stellate cells, Kupffer cells or liver sinusoidal endothelial cells.

In some aspects, a nucleic acid molecule formulated in a lipid nanoparticle of the present disclosure can comprise at least one genomic editing composition.

Gene editing compositions can comprise at least one nucleic acid molecule comprising at least one nucleic acid sequence encoding a DNA binding domain and a nucleic acid sequence encoding a nuclease protein or a nuclease domain thereof. The nucleic acid sequence encoding a nuclease protein or the sequence encoding a nuclease domain thereof can comprise a DNA sequence, an RNA sequence, or a combination thereof.

In some aspects, a genomic editing composition formulated in a lipid nanoparticle of the present disclosure can comprise a nucleic acid molecule comprising a nucleic acid sequence encoding a fusion protein, wherein the fusion protein comprises a nuclease-inactivated Cas (dCas) protein, or a nuclease domain thereof and a endonuclease protein, or a nuclease domain thereof.

In some aspects, a genomic editing composition formulated in a lipid nanoparticle of the present disclosure can comprise a nucleic acid molecule comprising a nucleic acid sequence encoding a fusion protein, wherein the fusion protein comprises (i) an inactivated Cas9 (dCas9) protein or an inactivated nuclease domain thereof, (ii) a Clo051 protein or a nuclease domain thereof. In some aspects, the fusion protein can further comprise at least one nuclear localization signal (NLS). In some aspects, the fusion protein can further comprise at least two NLSs. In some aspects, the nucleic acid molecule can comprise DNA, RNA or any combination thereof. In some aspects, the nucleic acid molecule can comprise RNA. Exemplary dCas9-Clo051 fusion proteins (referred to in the art as “Cas-CLOVER” proteins), and polynucleotide sequences encoding said dCas9-Clo051 fusion proteins, are described in detail in U.S. Patent Publication No. 2022/0042038, the contents of which is incorporated herein by reference in its entirety. Gene editing compositions, including Cas-CLOVER, and methods of using these compositions for gene editing are described in detail in U.S. Patent Publication Nos. 2017/0107541, 2017/0114149, 2018/0187185 and U.S. Pat. No. 10,415,024, the contents of each of which are incorporated herein by reference in their entireties.

In some aspects, a Cas-CLOVER protein can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 31.

In some aspects, a Clo051 protein or a nuclease domain can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 32.

In some aspects, an inactivated Cas9 (dCas9) protein or an inactivated nuclease domain thereof can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 33.

In some aspects, a genomic editing composition formulated in a lipid nanoparticle of the present disclosure can further comprise at least one guide molecule. In some aspects, the guide molecule can be a guide RNA (gRNA molecule).

Accordingly, the present disclosure provides any of the lipid nanoparticle compositions described herein, wherein the lipid nanoparticle comprises at least one genomic editing composition, wherein the at least one genomic editing composition comprises: a) a nucleic acid molecule comprising a nucleic acid sequence encoding a fusion protein, wherein the fusion protein comprises (i) an inactivated Cas9 (dCas9) protein or an inactivated nuclease domain thereof, (ii) a Clo051 protein or a nuclease domain thereof and b) at least one gRNA molecule. In some aspects, the fusion protein can further comprise at least one NLS. In some aspects, the at least one genomic editing composition can comprise at least two species of gRNA molecules.

The present disclosure provides a method of treating at least one disease in a subject, the method comprising administering to the subject at least one therapeutically effective amount of at least one composition of the present disclosure comprising at least one nucleic acid encoding a therapeutic protein.

The present disclosure provides a method of treating at least one disease in a subject, the method comprising administering at least one therapeutically effective amount of cells, wherein the cells have been contacted by at least one nanoparticle of the present disclosure comprising at least one nucleic acid encoding a therapeutic protein. The present disclosure provides a method of treating at least one disease in a subject, the method comprising administering at least one therapeutically effective amount of cells, wherein the cells have been genetically modified using the compositions and/or methods of the present disclosure.

In some aspects, the at least one disease can be a metabolic liver disorder (MLD). An MLD can include, but is not limited to, N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome), methylmalonic acidemia (MMA), progressive familia intrahepatic cholestasis type 1 (PFIC1), progressive familia intrahepatic cholestasis type 1 (PFIC2), progressive familia intrahepatic cholestasis type 1 (PFIC3) or any combination thereof.

In some aspects, the at least one disease can be a hemophilia disease. In some aspects, the hemophilia disease is hemophilia A. In some aspects, the hemophilia disease is hemophilia B. In some aspects, the hemophilia disease is hemophilia C.

In some aspects, the at least one disease can be a disease and/or disorder characterized by increased LDL-cholesterol. Accordingly, the present disclosure provides methods of decreasing LDL-cholesterol in a subject in need thereof.

In some aspects, a nucleic acid molecule formulated in a lipid nanoparticle of the present disclosure can comprise at least one transgene sequence. In some aspects, a transgene sequence can comprise a nucleotide sequence encoding at least one therapeutic protein. In some aspects, a transgene sequence can comprise a nucleotide sequence encoding at least one transposase.

In some aspects, a transgene sequence can comprise a nucleotide sequence encoding at least one transposon. In some aspects, a transposon can comprise a nucleotide sequence encoding at least one therapeutic protein. In some aspects, a transposon can comprise a nucleotide sequence encoding at least one therapeutic protein and at least one protomer sequence, wherein the at least one therapeutic protein is operatively linked to the at least one promoter sequence. In some aspects, a transposon can comprise at least one inverted terminal repeat (ITR). In some aspects, a transposon can comprise a first ITR and an at least second ITR. In some aspects, a transposon can comprise at least one insulator sequence. In some aspects, a transposon can comprise a first insulator sequence and an at least second insulator sequence. In some aspects, a transposon can comprise at least one sequence encoding at least one therapeutic protein. In some aspects, a transposon can comprise at least one 5′ UTR sequence. In some aspects, a transposon can comprise at least one 3′ UTR sequence. In some aspects, a transposon can comprise a first 3′ UTR sequence and an at least second 3′ UTR sequence. In some aspects, a transposon can comprise at least one polyA sequence.

In some aspects, a transposon can comprise at least one sequence encoding a therapeutic protein and a 3′ UTR sequence. In some aspects, a transposon can comprise, in the 5′ to 3′ direction, at least one sequence encoding a therapeutic protein and a 3′ UTR sequence. In some aspects, a transposon can comprise at least one sequence encoding a therapeutic protein, followed by a 3′ UTR sequence.

In some aspects, a transposon can comprise a first ITR, a first insulator sequence, at least one promoter sequence, at least one sequence encoding at least one therapeutic protein, a 3′ UTR sequence, a polyA sequence, a second insulator sequence, and a second ITR. In some aspects, a transposon can comprise, in the 5′ to 3′ direction, a first ITR, a first insulator sequence, at least one promoter sequence, at least one sequence encoding at least one therapeutic protein, a 3′ UTR sequence, a polyA sequence, a second insulator sequence, and a second ITR. In some aspects, a transposon can comprise, a first ITR, followed by a first insulator sequence, followed by at least one promoter sequence, followed by at least one promoter sequence, followed by at least one sequence encoding at least one therapeutic protein, followed by a 3′ UTR sequence, followed by a polyA sequence, followed by a second insulator sequence, followed by a second ITR.

In some aspects of the preceding transposons, an at least one sequence encoding at least one therapeutic protein can be a sequence encoding a FVIII-BDD polypeptide, wherein the FVIII-BDD polypeptide comprises the amino acid sequence of SEQ ID NO: 21. In some aspects, a sequence encoding a FVIII-BDD polypeptide can comprise the nucleic acid sequence of SEQ ID NO: 22.

In some aspects of the preceding transposons, a 3′ UTR sequence can comprise the nucleic acid sequence of SEQ ID NO: 27.

Accordingly, in a non-limiting example, a transposon can comprise at least one sequence encoding a therapeutic protein and a 3′ UTR sequence, wherein the at least one sequence encoding a therapeutic protein is a sequence encoding a FVIII-BDD polypeptide, wherein the FVIII-BDD polypeptide comprises the amino acid sequence of SEQ ID NO: 21, and wherein the 3′ UTR sequence comprises the nucleic acid sequence of SEQ ID NO: 27.

In some aspects, a transposon can comprise at least one sequence encoding a therapeutic protein, a first 3′ UTR sequence and a second 3′ UTR sequence. In some aspects, a transposon can comprise, in the 5′ to 3′ sequence, at least one sequence encoding a therapeutic protein, a first 3′ UTR sequence and a second 3′ UTR sequence. In some aspects, a transposon can comprise at least one sequence encoding a therapeutic protein, followed by a first 3′ UTR, followed by a second 3′ UTR sequence.

In some aspects, a transposon can comprise a first ITR, a first insulator sequence, at least one promoter sequence, at least one sequence encoding at least one therapeutic protein, a first 3′ UTR sequence, a second 3′ UTR sequence, a polyA sequence, a second insulator sequence, and a second ITR. In some aspects, a transposon can comprise, in the 5′ to 3′ direction, a first ITR, a first insulator sequence, at least one promoter sequence, at least one sequence encoding at least one therapeutic protein, a first 3′ UTR sequence, a second 3′ UTR sequence, a polyA sequence, a second insulator sequence, and a second ITR. In some aspects, a transposon can comprise, a first ITR, followed by a first insulator sequence, followed by at least one promoter sequence, followed by at least one promoter sequence, followed by at least one sequence encoding at least one therapeutic protein, followed by a first 3′ UTR sequence, followed by a second 3′ UTR sequence, followed by a polyA sequence, followed by a second insulator sequence, followed by a second ITR.

In some aspects of the preceding transposons, an at least one sequence encoding at least one therapeutic protein can be a sequence encoding a FVIII-BDD polypeptide, wherein the FVIII-BDD polypeptide comprises the amino acid sequence of SEQ ID NO: 21. In some aspects, to sequence encoding a FVIII-BDD polypeptide can comprise the nucleic acid sequence of SEQ ID NO: 22.

In some aspects of the preceding transposons, a first 3′ UTR sequence can be an AES 3′ UTR sequence, wherein the AES 3′ UTR sequence comprises the nucleic acid sequence of SEQ ID NO: 25.

In some aspects of the preceding transposons, a second 3′ UTR sequence can be a mtRNR1 3′ UTR sequence, wherein the mtRNR1 3′ UTR sequence comprises the nucleic acid sequence of SEQ ID NO: 26.

Accordingly, in a non-limiting example, a transposon can comprise at least one sequence encoding a therapeutic protein, a first 3′ UTR sequence and a second 3′ UTR sequence, wherein the at least one sequence encoding a therapeutic protein is a sequence encoding a FVIII-BDD polypeptide, wherein the FVIII-BDD polypeptide comprises the amino acid sequence of SEQ ID NO: 21, and wherein the first 3′ UTR sequence comprises the nucleic acid sequence of SEQ ID NO: 25 and the second 3′ UTR sequence comprises the nucleic acid sequence of SEQ ID NO: 26.

In some aspects, a transposon can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 28.

In some aspects, a transposon can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 34.

In some aspects, a transposon can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 35.

In some aspects, a therapeutic protein can comprise, consist essentially of, or consist of a methylmalonyl-CoA mutase (MUT1) polypeptide.

In some aspects, a therapeutic protein can comprise, consist essentially of, or consist of an ornithine transcarbamylase (OTC) polypeptide

In some aspects, a therapeutic protein can comprise, consist essentially of, or consist of a Factor VIII (FVIII) polypeptide. In some aspects, a FVIII polypeptide can be a FVIII polypeptide that is lacking the B-domain (hereafter referred to as a FVIII-BDD polypeptide). As would be appreciated by the skilled artisan, a Factor VIII-BDD polypeptide retains biological activity in vitro and in vivo (see Kessler et al. Haemophilia, 2005, 11(2): 84-91).

In some aspects, a FVIII-BDD polypeptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 21. In some aspects, a nucleic acid sequence that encodes for an FVIII-BDD polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 22 or 36.

In some aspects, a therapeutic protein can comprise, consist essentially of, or consist of a Factor IX (FIX) polypeptide. In some aspects, a FIX polypeptide can comprise a R338L mutation. As would be appreciated by the skilled artisan, the R338L mutation can be referred to as the Padua mutation (see VandenDriessche and Chuah, Molecular Therapy, 2018, Vol. 26, Issue 1, P14-16, the contents of which are incorporated herein by reference in their entireties).

The present disclosure provides methods of treating at least one disease in a subject, the methods comprising administering to the subject: a) at least one therapeutically effective amount of a composition comprising a nucleic acid molecule comprising a transposon, wherein the transposon comprises a nucleotide sequence encoding at least one therapeutic protein; and b) at least one therapeutically effective amount of a composition comprising a nucleic acid molecule comprising a nucleotide sequence encoding at least one transposase.

In some aspects of the preceding method, a composition comprising a nucleic acid molecule comprising a transposon can be a composition comprising at least one LNP of the present disclosure, wherein the LNP comprises at least one nucleic acid molecule comprising a transposon, wherein the transposon comprises a nucleotide sequence encoding at least one therapeutic protein. Accordingly, the present disclosure provides methods of treating at least one disease in a subject, the methods comprising administering to the subject: a) at least one therapeutically effective amount of LNPs of the present disclosure, wherein the LNPS comprise at least one nucleic acid molecule comprising a transposon, wherein the transposon comprises a nucleotide sequence encoding at least one therapeutic protein; and b) at least one therapeutically effective amount of a composition comprising a nucleic acid molecule comprising a nucleotide sequence encoding at least one transposase.

In some aspects of the preceding methods, a composition comprising a nucleic acid molecule comprising a nucleotide sequence encoding at least one transposase can be a composition comprising at least one LNP of the present disclosure, wherein the LNP comprises at least one nucleic acid molecule comprising a nucleotide sequence encoding at least one transposase. Accordingly, the present disclosure provides methods of treating at least one disease in a subject, the methods comprising administering to the subject: a) at least one therapeutically effective amount of a composition comprising a nucleic acid comprising a transposon, wherein the transposon comprises a nucleotide sequence encoding at least one therapeutic protein; and b) at least one therapeutically effective amount of LNPs of the present disclosure, wherein the LNPs comprise at least one nucleic acid comprising a nucleotide sequence encoding at least one transposase.

Additionally, the present disclosure also provides methods of treating at least one disease in a subject, the methods comprising administering to the subject: a) at least one therapeutically effective amount of LNPs of the present disclosure, wherein the LNPs comprise at least one nucleic acid molecule comprising a transposon, wherein the transposon comprises a nucleotide sequence encoding at least one therapeutic protein; and b) at least one therapeutically effective amount of LNPs of the present disclosure, wherein the LNPs comprise at least one nucleic acid molecule comprising a nucleotide sequence encoding at least one transposase.

In some aspects of the preceding methods, a composition comprising a nucleic acid molecule comprising a nucleotide sequence encoding at least one transposon can be a composition comprising Adeno-associated virus (AAV) viral vector particles comprising at least one nucleic acid molecule comprising a transposon, wherein the transposon comprises a nucleotide sequence encoding at least one therapeutic protein. Accordingly, the present disclosure provides methods of treating at least one disease in a subject, the methods comprising administering to the subject: a) at least one therapeutically effective amount of AAV viral vector particles comprising at least one nucleic acid molecule comprising a transposon, wherein the transposon comprises a nucleotide sequence encoding at least one therapeutic protein; and b) at least one therapeutically effective amount of a composition comprising a nucleic acid molecule comprising a nucleotide sequence encoding at least one transposase.

Additionally, the present disclosure provides methods of treating at least one disease in a subject, the methods comprising administering to the subject: a) at least one therapeutically effective amount AAV viral vector particles comprising at least one nucleic acid molecule comprising a transposon, wherein the transposon comprises a nucleotide sequence encoding at least one therapeutic protein; and b) at least one therapeutically effective amount of LNPs of the present disclosure, wherein the LNPs comprise at least one nucleic acid molecule comprising a nucleotide sequence encoding at least one transposase.

In some aspects of the preceding methods, a composition comprising a nucleic acid molecule comprising a nucleotide sequence encoding at least one transposase can be a composition comprising AAV viral vector particles comprising at least one nucleic acid molecule comprising a nucleotide sequence encoding at least one transposase. Accordingly, the present disclosure provides methods of treating at least one disease in a subject, the methods comprising administering to the subject: a) at least one therapeutically effective amount of a composition comprising a nucleic acid comprising a transposon, wherein the transposon comprises a nucleotide sequence encoding at least one therapeutic protein; and b) at least one therapeutically effective amount of AAV viral vector particles comprising at least one nucleic acid molecule comprising a nucleotide sequence encoding at least one transposase.

Additionally, the present disclosure provides methods of treating at least one disease in a subject, the methods comprising administering to the subject: a) at least one therapeutically effective amount of LNPs of the present disclosure, wherein the LNPs comprise at least one nucleic acid molecule comprising a transposon, wherein the transposon comprises a nucleotide sequence encoding at least one therapeutic protein; and b) at least one therapeutically effective amount of AAV viral vector particles comprising at least one nucleic acid molecule comprising a nucleotide sequence encoding at least one transposase.

In a non-limiting example, an AAV viral vector particles comprising at least one nucleic acid molecule comprising a transposon, wherein the transposon comprises a nucleotide sequence encoding at least one therapeutic protein, wherein the therapeutic protein is OTC, can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of SEQ ID NOs: 1-6.

In a non-limiting example, an AAV viral vector particles comprising at least one nucleic acid molecule comprising a transposon, wherein the transposon comprises a nucleotide sequence encoding at least one therapeutic protein, wherein the therapeutic protein is Factor VIII, can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of SEQ ID NOs: 8-14.

In a non-limiting example, an AAV viral vector particles comprising at least one nucleic acid molecule comprising a transposon, wherein the transposon comprises a nucleotide sequence encoding at least one therapeutic protein, wherein the therapeutic protein is Factor IX, can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of SEQ ID NOs: 15-20.

In a non-limiting example, an AAV viral vector particles comprising at least one nucleic acid molecule comprising a nucleotide sequence encoding a transposase, can comprise, can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 7.

In some aspects of the preceding methods, a composition comprising a nucleic acid molecule comprising a transposon, wherein the transposon comprises a nucleotide sequence encoding at least one therapeutic protein and a composition comprising a nucleic acid molecule comprising a nucleotide sequence encoding at least one transposase can be administered concurrently. In some aspects, a composition comprising a nucleic acid molecule comprising a transposon, wherein the transposon comprises a nucleotide sequence encoding at least one therapeutic protein and a composition comprising a nucleic acid molecule comprising a nucleotide sequence encoding at least one transposase can be administered sequentially. In some aspects, a composition comprising a nucleic acid molecule comprising a transposon, wherein the transposon comprises a nucleotide sequence encoding at least one therapeutic protein and a composition comprising a nucleic acid molecule comprising a nucleotide sequence encoding at least one transposase can be administered in temporal proximity.

As used herein, the term “temporal proximity” refers to that administration of one therapeutic composition (e.g., a composition comprising a transposon) occurs within a time period before or after the administration of another therapeutic composition (e.g., a composition comprising a transposase), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

In a non-limiting example, the present disclosure provides methods of treating a metabolic liver disorder in a subject, the methods comprising administering to the subject: a) at least one therapeutically effective amount of LNPs of the present disclosure, wherein the LNPs comprise at least one DNA molecule comprising a transposon, wherein the transposon comprises a nucleotide sequence encoding at least one therapeutic protein; and b) at least one therapeutically effective amount of LNPs of the present disclosure, wherein the LNPs comprise at least one RNA molecule comprising a nucleotide sequence encoding at least one transposase. In some aspects, the metabolic liver disorder can be Ornithine Transcarbamylase (OTC) Deficiency and the at least one therapeutic protein can comprise ornithine transcarbamylase (OTC) polypeptide. In some aspects, the metabolic liver disorder can be methylmalonic acidemia (MMA) and the at least one therapeutic protein can comprise a methylmalonyl-CoA mutase (MUT1) polypeptide.

In a non-limiting example, the present disclosure provides methods of treating a hemophilia disease in a subject, the methods comprising administering to the subject: a) at least one therapeutically effective amount of LNPs of the present disclosure, wherein the LNPs comprise at least one DNA molecule comprising a transposon, wherein the transposon comprises a nucleotide sequence encoding at least one therapeutic protein; and b) at least one therapeutically effective amount of LNPs of the present disclosure, wherein the LNPs comprise at least one RNA molecule comprising a nucleotide sequence encoding at least one transposase. In some aspects, the hemophilia disease can be hemophilia A and the at least one therapeutic protein can comprise Factor VIII. In some aspects, the hemophilia disease can be hemophilia B and the at least one therapeutic protein can comprise Factor IX.

In a non-limiting example, the present disclosure provides methods of treating a metabolic liver disorder in a subject, the methods comprising administering to the subject: a) at least one therapeutically effective amount AAV viral vector particles comprising at least one nucleic acid molecule comprising a transposon, wherein the transposon comprises a nucleotide sequence encoding at least one therapeutic protein; and b) at least one therapeutically effective amount of LNPs of the present disclosure, wherein the LNPs comprise at least one RNA molecule comprising a nucleotide sequence encoding at least one transposase. In some aspects, the metabolic liver disorder can be Ornithine Transcarbamylase (OTC) Deficiency and the at least one therapeutic protein can comprise ornithine transcarbamylase (OTC) polypeptide. In some aspects, the metabolic liver disorder can be methylmalonic acidemia (MMA) and the at least one therapeutic protein can comprise a methylmalonyl-CoA mutase (MUT1) polypeptide.

In a non-limiting example, the present disclosure provides methods of treating a hemophilia disease in a subject, the methods comprising administering to the subject: a) at least one therapeutically effective amount AAV viral vector particles comprising at least one nucleic acid molecule comprising a transposon, wherein the transposon comprises a nucleotide sequence encoding at least one therapeutic protein; and b) at least one therapeutically effective amount of LNPs of the present disclosure, wherein the LNPs comprise at least one RNA molecule comprising a nucleotide sequence encoding at least one transposase. In some aspects, the hemophilia disease can be hemophilia A and the at least one therapeutic protein can comprise Factor VIII. In some aspects, the hemophilia disease can be hemophilia B and the at least one therapeutic protein can comprise Factor IX.

The present disclosure provides methods of treating a disease and/or disorder characterized by increased LDL-cholesterol comprising administering to the subject at least one LNP of the present disclosure comprising a genomic editing composition, wherein the genomic editing composition comprises a nucleic acid molecule comprising a nucleic acid sequence encoding a fusion protein, wherein the fusion protein comprises (i) an inactivated Cas9 (dCas9) protein or an inactivated nuclease domain thereof, (ii) a Clo051 protein or a nuclease domain thereof. In some aspects, the fusion protein can be a Cas-CLOVER protein. In some aspects, the genomic editing composition can further comprise at least one species of guide RNA (gRNA) molecule targeting the pcsk9 gene. In some aspects, the genomic editing composition can further comprise at least two gRNA molecules targeting the pcsk9 gene. gRNA molecules targeting the pcsk9 gene can comprise, consist of, or consist essentially of a nucleic acid sequence at least 65%, 70%, 75%, 80%, 85%, 90%, 95%, 96%, 97%, 98%, 99% or 100% (or any percentage in between) identical to any one of SEQ ID NOs: 29-30.

The present disclosure provides methods of decreasing LDL-cholesterol in a subject in need thereof, the method comprising administering to the subject at least one LNP of the present disclosure comprising a genomic editing composition, wherein the genomic editing composition comprises a nucleic acid molecule comprising a nucleic acid sequence encoding a fusion protein, wherein the fusion protein comprises (i) an inactivated Cas9 (dCas9) protein or an inactivated nuclease domain thereof, (ii) a Clo051 protein or a nuclease domain thereof. In some aspects, the fusion protein can be a Cas-CLOVER protein. In some aspects, the genomic editing composition can further comprise at least one species of guide RNA (gRNA) molecule targeting the pcsk9 gene. In some aspects, the genomic editing composition can further comprise at least two gRNA molecules targeting the pcsk9 gene. gRNA molecules targeting the pcsk9 gene can comprise, consist of, or consist essentially of a nucleic acid sequence at least 65%, 70%, 75%, 80%, 85%, 90%, 95%, 96%, 97%, 98%, 99% or 100% (or any percentage in between) identical to any one of SEQ ID NOs: 29-30.

In some aspects of the treatment methods of the present disclosure, the administration of the at least one composition and/or nanoparticle of the present disclosure to a subject can result in the expression of an exogenous protein (e.g. a therapeutic protein, a transposase, etc.) in at least one organ and/or tissue in the subject.

In some aspects, the administration of the at least one composition and/or nanoparticle of the present disclosure results in the expression of the exogenous protein in at least about 10%, or at least about 15%, or at least bout 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% of the cells in the tissue and/or organ.

In some aspects, the administration of the at least one composition and/or nanoparticle of the present disclosure results in the expression of the exogenous protein in at least about 10%, or at least about 15%, or at least bout 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% of a specific subset or subsets of cells in the tissue and/or organ.

In some aspects, the administration of the at least one composition and/or nanoparticle of the present disclosure results in the expression of the exogenous protein for at least about 1 day, or at least about 2 days, or at least about 3 days, or at least about 4 days, or at least about 5 days, or at least about 6 days, or at least about 7 days, or at least about 8 days, or at least about 9 days, or at least about 10 days in the tissue and/or organ.

In some aspects, the administration of the at least one composition and/or nanoparticle of the present disclosure results in the expression of the exogenous protein for at least about 1 day, or at least about 2 days, or at least about 3 days, or at least about 4 days, or at least about 5 days, or at least about 6 days, or at least about 7 days, or at least about 8 days, or at least about 9 days, or at least about 10 days in a specific subset or subsets of cells in the tissue and/or organ.

In some aspects, the administration of the at least one composition and/or nanoparticle of the present disclosure results in the expression of the exogenous protein for no more than about 1 day, or no more than about 2 days, or no more than about 3 days, or no more than about 4 days, or no more than about 5 days, or no more than about 6 days, or no more than about 7 days, or no more than about 8 days, or no more than about 9 days, or no more than about 10 days in the tissue and/or organ.

In some aspects, the administration of the at least one composition and/or nanoparticle of the present disclosure results in the expression of the exogenous protein for no more than about 1 day, or no more than about 2 days, or no more than about 3 days, or no more than about 4 days, or no more than about 5 days, or no more than about 6 days, or no more than about 7 days, or no more than about 8 days, or no more than about 9 days, or no more than about 10 days in a specific subset or subsets of cells in the tissue and/or organ.

In some aspects, the percentage of cells that express an endogenous protein upon administration of a composition of the present disclosure can be at least about a 2 fold, or at least about a 3 fold, or at least about a 4 fold, or at least about a 5 fold, or at least about a 6 fold, or at least about a 7 fold, or at least about an 8 fold, or at least about a 9 fold, or at least about a 10 fold, or at last about a 15 fold, or at least about a 20 fold, or at least about a 25 fold, or at least about a 30 fold, or at least about a 50 fold increase as compared to the percentage of cells that express an endogenous protein upon administration of a composition comprising a control nanoparticle.

In some aspects, the tissue and/or organ can be the liver. In some aspects, the specific subset or subsets of cells can include, but are not limited to, hepatocytes, a hepatic stellate cells, Kupffer cells, liver sinusoidal endothelial cells or any combination thereof.

In some aspects, the administration of a composition comprising at least one lipid nanoparticle of the present disclosure is less toxic than the administration of a composition comprising at least one control lipid nanoparticle.

In some aspects, decreased toxicity can be manifested as an attenuation in the increase of the level of at least one liver enzyme following administration of the at least one lipid nanoparticle of the present disclosure. In some aspects, the at least one liver enzyme can be one or more of aspartate transaminase (AST), alanine transaminase (ALT) and alkaline phosphatase (ALP).

In some aspects, decreased toxicity can be manifested as an attenuation in the increase of the level of at least one proinflammatory cytokine following administration of the at least one lipid nanoparticle of the present disclosure. In some aspects, the at least one proinflammatory cytokine can be one or more of interleukin-6 (IL-6), interferon gamma (INF-G) and tumor necrosis factor alpha (TNF-a).

In some aspects, decreased toxicity can be manifested as an attenuation in a decrease in body weight following administration of the at least one lipid nanoparticle of the present disclosure.

In some aspects, a control lipid nanoparticle is a lipid nanoparticle that is otherwise identical except the cationic lipid is not a bioreducible ionizable cationic lipid.

In some aspects, a control lipid nanoparticle is a lipid nanoparticle that does not comprise a bioreducible ionizable cationic lipid.

In some aspects, a control lipid nanoparticle is a lipid nanoparticle that does not comprise ssPalmO-Ph-P4C2.

In some aspects, a control lipid nanoparticle is a lipid nanoparticle that is otherwise identical expect the cationic lipid is a lipid that is not ssPalmO-Ph-P4C2.

In some aspects, a control lipid nanoparticle is a lipid nanoparticle comprises a higher amount of a bioreducible ionizable cationic lipid.

In some aspects, a control lipid nanoparticle is a lipid nanoparticle comprises a lower amount of a bioreducible ionizable cationic lipid.

In some aspects, a control lipid nanoparticle comprises a lipid nanoparticle composition that has been previously disclosed in the art.

In some aspects, the control lipid nanoparticle is administered to the subject at the same dose as the lipid nanoparticles of the present disclosure.

In some aspects, the lipid nanoparticles of the present disclosure can be produced using a microfluidic-mixing platform. In some aspects, the microfluidic-mixing platform can be a non-turbulent microfluidic mixing platform.

In some aspects, a microfluidic-mixing platform can produce the lipid nanoparticles of the present disclosure by combining a miscible solvent phase comprising the lipid components of the nanoparticle and an aqueous phase comprising the lipid nanoparticle cargo (e.g. nucleic acid, DNA, mRNA, etc.) using a microfluidic device. In some aspects, the miscible solvent phase and the aqueous phase are mixed in the microfluidic device under laminar flow conditions that do not allow for immediate mixing of the two phases. As the two phases move under laminar flow in a microfluidic channel, microscopic features in the channel can allow for controlled, homogenous mixing to produce the lipid nanoparticles of the present disclosure.

In some aspects, the microfluidic-mixing platform can include, but are not limited to the NanoAssemblr® Spark (Precision NanoSystems), the NanoAssemblr® Ignite™ (Precision NanoSystems), the NanoAssemblr® Benchtop (Precision NanoSystems), the NanoAssemblr® Blaze (Precision NanoSystems) or the NanoAssemblr® GMP System (Precision NanoSystems).

In some aspects, the lipid nanoparticles of the present disclosure can be produced using a microfluidic-mixing platform, wherein the microfluidic mixing platform mixes at a rate of at least about 2.5 ml/min, or at least about 5 ml/min, or at least about 7.5 ml/min, or at least about 10 ml/min, or at least about 12.5 ml/min, or at least about 15 ml/min, or at least about 17.5 ml/min, or at least about 20 ml/min, or at least about 22.5 ml/min, or at least about 25 ml/min, or at least about 27.5 ml/min, or at least about 30 ml/min.

In some aspects, the lipid nanoparticles of the present disclosure can be produced using a T-mixer, wherein the T-mixer mixes at a rate of at least about 2.5 ml/min, or at least about 5 ml/min, or at least about 7.5 ml/min, or at least about 10 ml/min, or at least about 12.5 ml/min, or at least about 15 ml/min, or at least about 17.5 ml/min, or at least about 20 ml/min, or at least about 22.5 ml/min, or at least about 25 ml/min, or at least about 27.5 ml/min, or at least about 30 ml/min.

In some aspects, the lipid nanoparticles of the present disclosure can be produced using a microfluidic-mixing platform, wherein the microfluidic mixing platform mixes a miscible solvent phase and an aqueous phase at a ratio of about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10, solvent:aqueous, v:v.

In some aspects, the lipid nanoparticles of the present disclosure can be produced using a T-mixer, wherein the T-mixer mixes a miscible solvent phase and an aqueous phase at a ratio of about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10, solvent:aqueous, v:v.

piggyBac ITR Sequences

In some aspects, a nucleic acid molecule can comprise a piggyBac ITR sequence. In some aspects, a nucleic acid molecule can comprise a first piggyBac ITR sequence and a second piggyBac ITR sequence.

In some aspects, a piggyBac ITR sequence can comprise any piggyBac ITR sequence known in the art.

In some aspects of the methods of the present disclosure, a piggyBac ITR sequence, such as a first piggyBac ITR sequence and/or a second piggyBac ITR sequence can comprise, consist essentially of, or consist of a Sleeping Beauty transposon ITR, a Helraiser transposon ITR, a Tol2 transposon ITR, a TcBuster transposon ITR or any combination thereof

Insulator Sequences

In some aspects, an insulator sequence can comprise, consist essentially of, or consist of a nucleic acid sequence at least at least 65%, 70%, 75%, 80%, 85%, 90%, 95%, 96%, 97%, 98%, 99% or 100% (or any percentage in between) identical to any of the sequences put forth in SEQ ID NOs: 146-147.

Promoter Sequences

In some aspects, a nucleic acid molecule can comprise a promoter sequence. In some aspects, a promoter sequence can comprise any promoter sequence known in the art. In some aspects, a promoter sequence can comprise any liver-specific promoter sequence known in the art.

In some aspects, a promoter sequence can comprise a hybrid liver promoter (HLP). In some aspects, a promoter sequence can comprise an LP1 promoter. In some aspects, a promoter sequence can comprise a leukocyte-specific expression of the pp52 (LSP1) long promoter. In some aspects, a promoter sequence can comprise a thyroxine binding globulin (TBG) promoter.

In some aspects, a promoter sequence can comprise a wTBG promoter. In some aspects, a promoter sequence can comprise a hepatic combinatorial bundle (HCB) promoter. In some aspects, a promoter sequence can comprise a 2×ApoE-hAAT promoter. In some aspects, a promoter sequence can comprise a leukocyte-specific expression of the pp52 (LSP1) plus chimeric intron promoter. In some aspects, a promoter sequence can comprise a cytomegalovirus (CMV) promoter.

Transgene Sequences

In some aspects, a transgene sequence can comprise, consist essentially of, or consist of a nucleic acid sequence that encodes for a methylmalonyl-CoA mutase (MUT1) polypeptide. In some aspects, a nucleic acid sequence that encodes for an MUT1 polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 40-51.

In some aspects, a transgene sequence can comprise, consist essentially of, or consist of a nucleic acid sequence that encodes for an ornithine transcarbamylase (OTC) polypeptide. In some aspects, a nucleic acid sequence that encodes for an MUT1 polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 56-59.

In some aspects, a transgene sequence can comprise, consist essentially of, or consist of a nucleic acid sequence that encodes for an iCAS9 polypeptide.

In some aspects, a transgene sequence can comprise, consist essentially of, or consist of a nucleic acid sequence that encodes for a Factor VIII (FVIII) polypeptide. In some aspects, a FVIII polypeptide can be a FVIII polypeptide that is lacking the B-domain (hereafter referred to as a FVIII-BDD polypeptide).

In some aspects, a transgene sequence can comprise a nucleic acid sequence that encodes for a FVIII-BDD polypeptide. In some aspects, a transgene sequence can comprise a nucleic acid sequence that encodes for a FVIII-BDD polypeptide, wherein the FVIII-BDD polypeptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 21. In some aspects, a nucleic acid sequence that encodes for an FVIII-BDD polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 22.

In some aspects, a transgene sequence can comprise, consist essentially of, or consist of a nucleic acid sequence that encodes for a Factor IX (FIX) polypeptide. In some aspects, a FIX polypeptide can comprise a R338L mutation. As would be appreciated by the skilled artisan, the R338L mutation can be referred to as the Padua mutation. In some aspects, a nucleic acid sequence that encodes for a FIX polypeptide can comprise, consist essentially of, or consist of a nucleic acid sequence at least 65%, 70%, 75%, 80%, 85%, 90%, 95%, 96%, 97%, 98%, 99% or 65.

In some aspects, a transgene sequence can be codon optimized according to methods known in the art.

In some aspects, an at least one transgene sequence can be operatively linked to at least one promoter sequence present in the same polynucleotide.

Therapeutic Protein

In some aspects, a therapeutic protein can comprise, consist essentially of, or consist of a methylmalonyl-CoA mutase (MUT1) polypeptide. In some aspects, a MUT1 polypeptide comprises, consists essentially of or consist of an amino acid sequence at least 65%, 70%, 75%, 80%, 85%, 90%, 95%, 96%, 97%, 98%, 99% or 100% (or any percentage in between) identical to any one of SEQ ID NOs: 52-55.

In some aspects, a therapeutic protein can comprise, consist essentially of, or consist of an ornithine transcarbamylase (OTC) polypeptide. In some aspects, an OTC polypeptide comprises, consists essentially of or consist of an amino acid sequence at least 65%, 70%, 75%, 80%, 85%, 90%, 95%, 96%, 97%, 98%, 99% or 100% (or any percentage in between) identical to any one of SEQ ID NOs: 60-63.

In some aspects, a therapeutic protein can comprise, consist essentially of, or consist of a Factor VIII (FVIII) polypeptide. In some aspects, a FVIII polypeptide can be a FVIII polypeptide that is lacking the B-domain (hereafter referred to as a FVIII-BDD polypeptide).

In some aspects, a therapeutic protein can comprise, consist essentially of, or consist of a Factor IX (FIX) polypeptide. In some aspects, a FIX polypeptide can comprise a R338L mutation. As would be appreciated by the skilled artisan, the R338L mutation can be referred to as the Padua mutation. In some aspects, a FIX polypeptide comprises, consists essentially of or consist of an amino acid sequence at least 65%, 70%, 75%, 80%, 85%, 90%, 95%, 96%, 97%, 98%, 99% or 100% (or any percentage in between) identical to any one of SEQ ID NOs: 64.

3′ UTR Sequence

In some aspects, a 3′ UTR sequence can be an AES 3′ UTR sequence. An AES 3′ UTR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 25.

In some aspects, a 3′ UTR sequence can be a mtRNR1 3′ UTR sequence. A mtRNR1 3′ UTR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 26.

In some aspects, a 3′ UTR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 27.

polyA Sequences

In some aspects, a nucleic acid molecule can comprise a polyA sequence. In some aspects, a polyA sequence can comprise any polyA sequence known in the art.

Self-Cleaving Peptide Sequence

In some aspects, a nucleic acid molecule can comprise a self-cleaving peptide sequence. In some aspects, a self-cleaving peptide sequence can comprise any self-cleaving peptide sequence known in the art. In some aspects, a self-cleaving peptide sequence can comprise an 2A self-cleaving peptide sequence known in the art. Non-limiting examples of self-cleaving peptides include a T2A peptide, GSG-T2A peptide, an E2A peptide, a GSG-E2A peptide, an F2A peptide, a GSG-F2A peptide, a P2A peptide, or a GSG-P2A peptide.

In some aspects, a self-cleaving peptide sequence can comprise a nucleic acid sequence that encodes for a T2A peptide.

In some aspects, a self-cleaving peptide sequence can comprise a nucleic acid sequence that encodes for a GSG-T2A peptide.

In some aspects, a self-cleaving peptide sequence can comprise a nucleic acid sequence that encodes for an E2A peptide.

In some aspects, a self-cleaving peptide sequence can comprise a nucleic acid sequence that encodes for a GSG-E2A peptide.

In some aspects, a self-cleaving peptide sequence can comprise a nucleic acid sequence that encodes for a F2A peptide.

In some aspects, a self-cleaving peptide sequence can comprise a nucleic acid sequence that encodes for a GSG-F2A peptide.

In some aspects, a self-cleaving peptide sequence can comprise a nucleic acid sequence that encodes for a P2A peptide.

In some aspects, a self-cleaving peptide sequence can comprise a nucleic acid sequence that encodes for a GSG-P2A peptide.

Transposition Systems

In some aspects, a nucleic acid molecule can comprise a transposon or a nanotransposon comprising: a first nucleic acid sequence comprising: (a) a first inverted terminal repeat (ITR) or a sequence encoding a first ITR, (b) a second ITR or a sequence encoding a second ITR, and (c) an intra-ITR sequence or a sequence encoding an intra-ITR, wherein the intra-ITR sequence comprises a transposon sequence or a sequence encoding a transposon.

In some aspects, a nucleic acid molecule can comprise a transposon or a nanotransposon comprising: a first nucleic acid sequence comprising: (a) a first inverted terminal repeat (ITR) or a sequence encoding a first ITR, (b) a second ITR or a sequence encoding a second ITR, and (c) an intra-ITR sequence or a sequence encoding an intra-ITR, wherein the intra-ITR sequence comprises a transposon sequence or a sequence encoding a transposon, and a second nucleic acid sequence comprising an inter-ITR sequence or a sequence encoding an inter-ITR, wherein the length of the inter-ITR sequence is equal to or less than 700 nucleotides.

The transposon or nanotransposon of the disclosure comprises a nucleotide sequence encoding a therapeutic protein. The transposon or nanotransposon can be a plasmid DNA transposon comprising a nucleotide sequence encoding a therapeutic protein flanked by two cis-regulatory insulator elements. The transposon or nanotransposon can further comprises a plasmid comprising a sequence encoding a transposase. The sequence encoding the transposase may be a DNA sequence or an RNA sequence. Preferably, the sequence encoding the transposase is an mRNA sequence.

The transposon or nanotransposon of the present disclosure can be a piggyBac™ (PB) transposon. In some aspects when the transposon is a PB transposon, the transposase is a piggyBac™ (PB) transposase a piggyBac-like (PBL) transposase or a Super piggyBac™ (SPB or sPB) transposase. Preferably, the sequence encoding the SPB transposase is an mRNA sequence.

Non-limiting examples of PB transposons and PB, PBL and SPB transposases are described in detail in U.S. Pat. Nos. 6,218,182; 6,962,810; 8,399,643 and PCT Publication No. WO 2010/099296.

The PB, PBL and SPB transposases recognize transposon-specific inverted terminal repeat sequences (ITRs) on the ends of the transposon, and inserts the contents between the ITRs at the sequence 5′-TTAT-3′ within a chromosomal site (a TTAT target sequence) or at the sequence 5′-TTAA-3′ within a chromosomal site (a TTAA target sequence). The target sequence of the PB or PBL transposon can comprise or consist of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. The PB or PBL transposon system has no payload limit for the genes of interest that can be included between the ITRs.

Exemplary amino acid sequence for one or more PB, PBL and SPB transposases are disclosed in U.S. Pat. Nos. 6,218,185; 6,962,810 and 8,399,643.

The PB or PBL transposase can comprise or consist of an amino acid sequence having an amino acid substitution at two or more, at three or more or at each of positions 30, 165, 282, or 538. The transposase can be a SPB transposase that comprises or consists of the amino acid sequence wherein the amino acid substitution at position 30 can be a substitution of a valine (V) for an isoleucine (I), the amino acid substitution at position 165 can be a substitution of a serine (S) for a glycine (G), the amino acid substitution at position 282 can be a substitution of a valine (V) for a methionine (M), and the amino acid substitution at position 538 can be a substitution of a lysine (K) for an asparagine (N).

In certain aspects wherein the transposase comprises the above-described mutations at positions 30, 165, 282 and/or 538, the PB, PBL and SPB transposases can further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 are described in more detail in PCT Publication No. WO 2019/173636 and PCT/US2019/049816.

The PB, PBL or SPB transposases can be isolated or derived from an insect, vertebrate, crustacean or urochordate as described in more detail in PCT Publication No. WO 2019/173636 and PCT/US2019/049816. In preferred aspects, the PB, PBL or SPB transposases is be isolated or derived from the insect Trichoplusia ni (GenBank Accession No. AAA87375) or Bombyx mori (GenBank Accession No. BAD11135).

A hyperactive PB or PBL transposase is a transposase that is more active than the naturally occurring variant from which it is derived. In a preferred aspect, a hyperactive PB or PBL transposase is isolated or derived from Bombyx mori or Xenopus tropicalis. Examples of hyperactive PB or PBL transposases are disclosed in U.S. Pat. Nos. 6,218,185; 6,962,810, 8,399,643 and WO 2019/173636. A list of hyperactive amino acid substitutions is disclosed in U.S. Pat. No. 10,041,077.

In some aspects, the PB or PBL transposase is integration deficient. An integration deficient PB or PBL transposase is a transposase that can excise its corresponding transposon, but that integrates the excised transposon at a lower frequency than a corresponding wild type transposase. Examples of integration deficient PB or PBL transposases are disclosed in U.S. Pat. Nos. 6,218,185; 6,962,810, 8,399,643 and WO 2019/173636. A list of integration deficient amino acid substitutions is disclosed in U.S. Pat. No. 10,041,077.

In some aspects, the PB or PBL transposase is fused to a nuclear localization signal. Examples of PB or PBL transposases fused to a nuclear localization signal are disclosed in U.S. Pat. Nos. 6,218,185; 6,962,810, 8,399,643 and WO 2019/173636.

In some aspects, a sPB protein can comprise, consist essentially of, or consist of an amino acid sequence at least 65%, 70%, 75%, 80%, 85%, 90%, 95%, 96%, 97%, 98%, 99% or 100% (or any percentage in between) identical to any one of SEQ ID NOs: 37-39.

A transposon or nanotransposon of the present disclosure can be a Sleeping Beauty transposon. In some aspects, when the transposon is a Sleeping Beauty transposon, the transposase is a Sleeping Beauty transposase (for example as disclosed in U.S. Pat. No. 9,228,180) or a hyperactive Sleeping Beauty (SB100×) transposase.

A transposon or nanotransposon of the present disclosure can be a Helraiser transposon. An exemplary Helraiser transposon includes Helibat1. In some aspects, when the transposon is a Helraiser transposon, the transposase is a Helitron transposase (for example, as disclosed in WO 2019/173636).

A transposon or nanotransposon of the present disclosure can be a Tol2 transposon. In some aspects, when the transposon is a Tol2 transposon, the transposase is a Tol2 transposase (for example, as disclosed in WO 2019/173636).

A transposon or nanotransposon of the present disclosure can be a TcBuster transposon. In some aspects, when the transposon is a TcBuster transposon, the transposase is a TcBuster transposase or a hyperactive TcBuster transposase (for example, as disclosed in WO 2019/173636). The TcBuster transposase can comprise or consist of a naturally occurring amino acid sequence or a non-naturally occurring amino acid sequence. The polynucleotide encoding a TcBuster transposase can comprise or consist of a naturally occurring nucleic acid sequence or a non-naturally occurring nucleic acid sequence.

In some aspects, a mutant TcBuster transposase comprises one or more sequence variations when compared to a wild type TcBuster transposase as described in more detail in PCT Publication No. WO 2019/173636 and PCT/US2019/049816.

The cell delivery compositions (e.g., transposons) disclosed herein can comprise a nucleic acid molecule encoding a therapeutic protein or therapeutic agent. Examples of therapeutic proteins include those disclosed in PCT Publication No. WO 2019/173636 and PCT/US2019/049816.

Cells and Modified Cells of the Disclosure

Cells and modified cells of the disclosure can be mammalian cells. Preferably, the cells and modified cells are human cells. In one aspect, the cells targeted for modification using the LNP compositions of the present disclosure are hepatocytes, a hepatic stellate cells, Kupffer cells or liver sinusoidal endothelial cells. In one embodiment, the LNP compositions comprise at least one mRNA molecule encoding a transposase and the modified cells are generated in vivo. In one embodiment, the LNP compositions comprise at least one DNA molecule encoding a transposon and the modified cells are generated in vivo. In one embodiment, the transposon comprises a nucleotide sequence encoding a therapeutic gene operatively linked to a liver-specific promoter.

Cells and modified cells of the disclosure can be somatic cells. Cells and modified cells of the disclosure can be differentiated cells. Cells and modified cells of the disclosure can be autologous cells or allogenic cells. Allogeneic cells are engineered to prevent adverse reactions to engraftment following administration to a subject. Allogeneic cells may be any type of cell. Allogenic cells can be stem cells or can be derived from stem cells. Allogeneic cells can be differentiated somatic cells.

Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the composition as well known in the art or as described herein.

For example, the disclosed LNP compositions of the present disclosure can further comprise a diluent. In some compositions, the diluent can be phosphate buffered saline (“PBS”). In some compositions, the diluent can be sodium acetate.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers (e.g., jet nebulizer, ultrasonic nebulizer), dry powder generators, sprayers, and the like. All such devices can use formulations suitable for the administration for the dispensing of a composition or pharmaceutical composition described herein in an aerosol. Such aerosols can be comprised of either solutions (both aqueous and non-aqueous) or solid particles. Additionally, a spray including a composition or pharmaceutical composition described herein can be produced by forcing a suspension or solution of at least one protein scaffold through a nozzle under pressure. In a metered dose inhaler (MDI), a propellant, a composition or pharmaceutical composition described herein, and any excipients or other additives are contained in a canister as a mixture including a liquefied compressed gas. Actuation of the metering valve releases the mixture as an aerosol. A more detailed description of pulmonary administration, formulations and related devices is disclosed in PCT Publication No. WO 2019/049816.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695). A more detailed description of mucosal administration and formulations is disclosed in PCT Publication No. WO 2019/049816.

For transdermal administration, a composition or pharmaceutical composition disclosed herein is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599). A more detailed description of transdermal administration, formulations and suitable devices is disclosed in PCT Publication No. WO 2019/049816.

It can be desirable to deliver the disclosed compounds to the subject over prolonged periods of time, for example, for periods of one week to one year from a single administration. Various slow release, depot or implant dosage forms can be utilized.

Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired.

As a non-limiting example, treatment of humans or animals can be provided as a one-time or periodic dosage of the compositions or pharmaceutical compositions disclosed herein about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

In aspects where the compositions to be administered to a subject in need thereof are modified cells as disclosed herein, the cells can be administered between about 1×10³ and 1×10¹⁵ cells; 1×10³ and 1×10¹⁵ cells, about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In an aspect the cells are administered between about 5×10⁶ and 25×10⁶ cells.

A more detailed description of pharmaceutically acceptable excipients, formulations, dosages and methods of administration of the disclosed compositions and pharmaceutical compositions is disclosed in PCT Publication No. WO 2019/04981.

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In an aspect, the subject is a mammal. Preferably, the subject is human. The terms “subject” and “patient” are used interchangeably herein.

Any use or method of the present disclosure can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one chemotherapeutic agent (e.g., an alkylating agent, an a mitotic inhibitor, a radiopharmaceutical).

In some aspects, the subject does not develop graft vs. host (GvH) and/or host vs. graft (HvG) following administration. In an aspect, the administration is systemic. Systemic administration can be any means known in the art and described in detail herein. Preferably, systemic administration is by an intravenous injection or an intravenous infusion. In an aspect, the administration is local. Local administration can be any means known in the art and described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously. In some aspects, where the composition is autologous cells or allogeneic cells, the dose is an amount sufficient for the cells to engraft and/or persist for a sufficient time to treat the disease or disorder.

In some aspects of the methods of treatment described herein, the treatment can be modified or terminated. Specifically, in aspects where the composition used for treatment comprises an inducible proapoptotic polypeptide, apoptosis may be selectively induced in the cell by contacting the cell with an induction agent. A treatment may be modified or terminated in response to, for example, a sign of recovery or a sign of decreasing disease severity/progression, a sign of disease remission/cessation, and/or the occurrence of an adverse event. In some aspects, the method comprises the step of administering an inhibitor of the induction agent to inhibit modification of the cell therapy, thereby restoring the function and/or efficacy of the cell therapy (for example, when a sign or symptom of the disease reappear or increase in severity and/or an adverse event is resolved).

Nucleic Acid Molecules

Nucleic acid molecules of the disclosure encoding a therapeutic protein can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the disclosure can include nucleic acid molecules comprising an open reading frame (ORF), optionally, with one or more introns, e.g., but not limited to, at least one specified enzymatically active portion of a therapeutic protein; nucleic acid molecules comprising the coding sequence for a therapeutic protein and nucleic acid molecules which comprise a nucleotide sequence substantially different from those described above but which, due to the degeneracy of the genetic code, still encode the therapeutic protein as described herein and/or as known in the art. Of course, the genetic code is well known in the art. Thus, it would be routine for one skilled in the art to generate such degenerate nucleic acid variants that code for a specific protein scaffold of the present disclosure. See, e.g., Ausubel, et al., supra, and such nucleic acid variants are included in the present disclosure.

As indicated herein, nucleic acid molecules of the disclosure which comprise a nucleic acid molecule encoding a therapeutic protein can include, but are not limited to, those encoding the amino acid sequence of an enzymatically active fragment of a therapeutic protein, by itself; the coding sequence for the entire a therapeutic protein or a portion thereof; the coding sequence for a therapeutic protein, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding a therapeutic protein can be fused to a marker sequence, such as a sequence encoding a peptide that facilitates purification of the fused therapeutic protein.

Construction of Nucleic Acids

The isolated nucleic acids of the disclosure can be made using (a) recombinant methods, (b) synthetic techniques, (c) purification techniques, and/or (d) combinations thereof, as well-known in the art.

The nucleic acids can conveniently comprise sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

Recombinant Methods for Constructing Nucleic Acids

The isolated nucleic acid compositions of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries are well known to those of ordinary skill in the art. (See, e.g., Ausubel, supra; or Sambrook, supra).

Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

Synthetic Methods for Constructing Nucleic Acids

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

Recombinant Expression Cassettes

The disclosure further provides recombinant expression cassettes comprising a nucleic acid of the disclosure. A nucleic acid sequence of the disclosure, for example, a cDNA or a genomic sequence encoding a protein scaffold of the disclosure, can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some aspects, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

Expression Vectors and Host Cells

The disclosure also relates to vectors that include isolated nucleic acid molecules of the disclosure, host cells that are genetically engineered with the recombinant vectors, and the production of at least one therapeutic protein by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124(8):1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCF, RAD51C, GCS, MDR1, ALDH1, NKX2.2, or any combination thereof.

At least one protein scaffold of the disclosure can be expressed in a modified form, such as a fusion protein, and can include not only secretion signals, but also additional heterologous functional regions. For instance, a region of additional amino acids, particularly charged amino acids, can be added to the N-terminus of a protein scaffold to improve stability and persistence in the host cell, during purification, or during subsequent handling and storage. Also, peptide moieties can be added to a protein scaffold of the disclosure to facilitate purification. Such regions can be removed prior to final preparation of a protein scaffold or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid molecule encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a preferred aspect, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

Amino Acid Codes

The amino acids that make up protein scaffolds of the disclosure are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994). A therapeutic protein of the disclosure can include one or more amino acid substitutions, deletions or additions, from spontaneous or mutations and/or human manipulation, as specified herein. Amino acids in a therapeutic protein of the disclosure that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one neutralizing activity. Sites that are critical for maintaining the activity of the therapeutic protein can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

As those of skill will appreciate, the disclosure includes at least one biologically active therapeutic protein of the disclosure. Biologically active therapeutic protein have a specific activity at least 20%, 30%, or 40%, and, preferably, at least 50%, 60%, or 70%, and, most preferably, at least 80%, 90%, or 95%-99% or more of the specific activity of the native (non-synthetic), endogenous or related and known protein scaffold. Methods of assaying and quantifying measures of enzymatic activity and substrate specificity are well known to those of skill in the art.

In another aspect, the disclosure relates to therapeutic proteins and fragments, as described herein, which are modified by the covalent attachment of an organic moiety. Such modification can produce a protein scaffold fragment with improved pharmacokinetic properties (e.g., increased in vivo serum half-life). The organic moiety can be a linear or branched hydrophilic polymeric group, fatty acid group, or fatty acid ester group. In particular aspect, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified therapeutic proteins and fragments of the disclosure can comprise one or more organic moieties that are covalently bonded, directly or indirectly, to the antibody. Each organic moiety that is bonded to a protein scaffold or fragment of the disclosure can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, a therapeutic protein modified by the covalent attachment of polylysine is encompassed by the disclosure. Hydrophilic polymers suitable for modifying therapeutic proteins of the disclosure can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the therapeutic protein of the disclosure has a molecular weight of about 800 to about 150,000 Daltons as a separate molecular entity. For example, PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying therapeutic proteins of the disclosure can be saturated or can contain one or more units of unsaturation. Fatty acids that are suitable for modifying protein scaffolds of the disclosure include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-Δ9-octadecanoate (C18, oleate), all cis-Δ5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified therapeutic proteins and fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH2)3-, —NH—(CH2)6-NH—, —(CH2)2-NH— and —CH2-O—CH2-CH2-O-CH2-CH2-O—CH—NH—.

Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified therapeutic proteins of the disclosure can be produced by reacting a protein scaffold or fragment with a modifying agent. For example, the organic moieties can be bonded to the protein scaffold in a non-site specific manner by employing an amine-reactive modifying agent, for example, an NHS ester of PEG. Modified therapeutic proteins and fragments comprising an organic moiety that is bonded to specific sites of a protein scaffold of the disclosure can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif (1996).

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

It is to be understood that the compounds described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

The term “epitope” refers to an antigenic determinant of a polypeptide. An epitope could comprise three amino acids in a spatial conformation, which is unique to the epitope. Generally, an epitope consists of at least 4, 5, 6, or 7 such amino acids, and more usually, consists of at least 8, 9, or 10 such amino acids. Methods of determining the spatial conformation of amino acids are known in the art, and include, for example, x-ray crystallography and two-dimensional nuclear magnetic resonance.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

The term “scFv” refers to a single-chain variable fragment. scFv is a fusion protein of the variable regions of the heavy (VH) and light chains (VL) of immunoglobulins, connected with a linker peptide. The linker peptide may be from about 5 to 40 amino acids or from about 10 to 30 amino acids or about 5, 10, 15, 20, 25, 30, 35, or 40 amino acids in length. Single-chain variable fragments lack the constant Fc region found in complete antibody molecules, and, thus, the common binding sites (e.g., Protein G) used to purify antibodies. The term further includes a scFv that is an intrabody, an antibody that is stable in the cytoplasm of the cell, and which may bind to an intracellular protein.

The term “single domain antibody” means an antibody fragment having a single monomeric variable antibody domain which is able to bind selectively to a specific antigen. A single-domain antibody generally is a peptide chain of about 110 amino acids long, comprising one variable domain (VH) of a heavy-chain antibody, or of a common IgG, which generally have similar affinity to antigens as whole antibodies, but are more heat-resistant and stable towards detergents and high concentrations of urea. Examples are those derived from camelid or fish antibodies. Alternatively, single-domain antibodies can be made from common murine or human IgG with four chains.

The terms “specifically bind” and “specific binding” as used herein refer to the ability of an antibody, an antibody fragment or a nanobody to preferentially bind to a particular antigen that is present in a homogeneous mixture of different antigens. In some aspects, a specific binding interaction will discriminate between desirable and undesirable antigens in a sample. In some aspects, more than about ten- to 100-fold or more (e.g., more than about 1000- or 10,000-fold). “Specificity” refers to the ability of an immunoglobulin or an immunoglobulin fragment, such as a nanobody, to bind preferentially to one antigenic target versus a different antigenic target and does not necessarily imply high affinity.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table 1.

TABLE 1
Conservative Substitutions I

| Side chain characteristics | | Amino Acid |
|---|---|---|
| Aliphatic | Non-polar | G A P I L V F |
| | Polar-uncharged | C S T M N Q |
| | Polar-charged | D E K R |
| Aromatic | | H F W Y |
| Other | | N Q D E |

Alternately, conservative amino acids can be grouped as described in Lehninger, (Biochemistry, Second Edition; Worth Publishers, Inc. NY, N.Y. (1975), pp. 71-77) as set forth in Table 2.

TABLE 2
Conservative Substitutions II

| Side Chain Characteristic | | Amino Acid |
|---|---|---|
| Non-polar | Aliphatic: | A L I V P |
| (hydrophobic) | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table 3.

TABLE 3
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### Example 1—Preparation of mRNA for LNP Compositions

The DNA plasmid pRT-HA-SPB-CC-AG encodes Super piggyBac transposase comprising a 5′-hemmagglutinin tag corresponding to amino acids 98-106 (“HA-SPB”). This plasmid was used as a template for in vitro transcription reactions to produce mRNA encoding HA-SPB further comprising a 5′-CAP.

Briefly, approximately 10 ug of supercoiled pRT-HA-SPB-CC-AG was added to a 1.5 ml Eppendorf tube comprising 1× CutSmart Buffer, 200 units of the restriction enzyme Spel (New England Biolabs, Cat #R31331) in 100 μl total volume. The plasmid DNA was linearized by incubating at 37° C. overnight to ensure complete digestion.

The linearized plasmid was purified using a DNA QIAquick PCR purification kit (Qiagen, Cat #28104) according to the manufacturer's instructions, and eluting the purified DNA in 40 μl of nuclease free water. The DNA concentration of the eluate was determined using a NanoDrop microvolume spectrophotometer (ThermoFisher) in accordance with the manufacturer's instructions.

The purified plasmid was used as a DNA template to produce mRNA using the in vitro transcription mMESSAGE mMACHINE T7 Transcription Kit (ThermoFisher, Cat #AM1344) in accordance with the manufacturer's instructions. Briefly, 100 mM stocks of the nucleotides GTP, ATP, UTP, and 5MeC (5-Methylcytidine-5′-Triphosphate) (TriLink #N=1014) and CleanCap reagent AG (m7G(5′)ppp(5′)(2′OMeA)pG; Trilink #N-7113) were prepared. 15 μl each of ATP, UTP and 5MeC and 12 uL each of GTP and CleanCap Reagent AG were added to a 100 μl total volume.

Approximately 1.67 μg of linear pRT-HA-SPB-CC-AG DNA, 20 μl of 10× T7 Transcription Buffer and 20 μl of T7 RNA polymerase mix was added to a 1.5 ml Eppendorf tube (200 μl final volume), and the tube was incubated at 37° C. for 3 hours. A 10 μl aliquot of TURBO DNase enzyme (ThermoFisher) was added and the tube further incubated at 37° C. for 15 min to degrade the DNA template.

A poly(A) tail was added to the 3′end of the 5′-CleanCap®-HA-SPB mRNA using reagents and procedures supplied in the mMESSAGE mMACHINE T7 Transcription kit (ThermoFisher Cat #AM1344).

The 5′-CleanCap®-HA-SPB-poly(A)-5MeC mRNA was purified using a RNeasy Midi Purification Kit (Qiagen, Cat #75144) according to the manufacturer's instructions. Briefly, a 3.5 ml solution of Buffer RLT was freshly prepared using 35 μl of 2-mercaptoethanol and combined with 2.5 ml of 100% ethanol, and the final mRNA product was eluted from the column using 300 μl of nuclease-free water. The average mRNA yield from this process is about 600-800 jig.

### Example 2—Preparation of LNPs of Present Disclosure Comprising mRNA and In Vivo Screening

A. Preparation

The following is a nonlimiting example that provides a exemplary methods for formulating a plurality of multi-component LNP compositions comprising a bioreducible ionizable cationic lipid and mRNA.

To formulate the LNPs, various percentages of the bioreducible ionizable cationic lipid ssPalmO-Ph-P4C2, the phospholipid DOPE, the structural lipid cholesterol (Chol) and 1,2-dimyristoyl-sn-glycerol methoxypolyethylene glycol (DMG-PEG2000; Avanti Polar Lipids, Alabaster, Alabama, USA) were combined to prepare LNP compositions.

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temp and then placed on a hot plate maintained at a temperature range of 50-55° C. Subsequently, the hot lipid stock solutions were combined to yield desired final mol percentages. A subset of the LNP compositions are shown in Tables 4a and 4b.

TABLE 4a

| | | | | | Total | |
|---|---|---|---|---|---|---|
| LNP | ssPalmO—Ph—P4C2 | DOPE | Chol | PEG-DMG | Lipid | Lipid:RNA |
| ID | (% moles) | (% moles) | (% moles) | (% moles) | (mM) | (w/w) |
| 3 | 28 | 10 | 60 | 2 | 10 | 10 |
| 9 | 28 | 10 | 60 | 2 | 10 | 100 |
| 11 | 60 | 10 | 29.5 | 0.5 | 10 | 100 |
| 13 | 32.92 | 32.92 | 32.92 | 1.25 | 17.5 | 55 |
| 24 | 10 | 60 | 29.5 | 0.5 | 25 | 100 |
| 28 | 60 | 10 | 28 | 2 | 25 | 100 |
| 29 | 60 | 10 | 28.75 | 1.25 | 25 | 100 |

TABLE 4b

| | | | | | Total | |
|---|---|---|---|---|---|---|
| LNP | ssPalmO—Ph—P4C2 | DOPE | Chol(% | PEG-DMG(% | Lipid | Lipid:RNA |
| ID | (% moles) | (% moles) | moles) | moles) | (mM) | (w/w) |
| 0 | 55 | 5 | 40 | 1 | 25 | 100:1 |

A 1 mg/ml solution of the 5′-CleanCap-5MeC-fLuciferase mRNA (TriLink Biotech) to be incorporated into the LNPs was added to 150 mM sodium acetate buffer (pH 5.2) to form a stock solution and kept on ice. The lipid phase was mixed with the aqueous mRNA phase inside a microfluidic chip using a NanoAssemblr® instrument (Precision Nanosystems, Vancouver, BC, Canada) according to the manufacturer's instructions to form LNP compositions comprising encapsulated mRNAs. Nanoassemblr® process parameters for mRNA encapsulation are shown in the Table 5.

TABLE 5

| Total flow rate | Lipid phase:aqueous |
|---|---|
| (ml/min) | (RNA) phase (v/v) |
| 20 | 1:3 |

The resultant mRNA LNP compositions were then transferred to a Repligen Float-A-Lyzer dialysis device—having a molecular weight cut off (MWCO) of 8-10 kDa (Spectrum Chemical Mfg. Corp, CA, USA) and processed by dialysis against phosphate buffered saline (PBS) (dialysate:dialysis buffer volume at least 1:200 v/v), pH 7.4 overnight at 4° C. (or alternatively room temperature for at least 4 hours), to remove the 25% ethanol and achieve a complete buffer exchange. In some experiments the LNPs were further concentrated in an Amicon® Ultra-4 centrifugal filter unit, MWCO-30 kDa (Millipore Sigma, USA) spun at ˜4100×g in an ultracentrifuge. The mRNA LNPs were then stored at 4° C. until further use.

The average particle size diameter of the LNPs was approximately 70 nm.

B. In Vivo Screening

Adult female BALB/C mice (n=2/group) were intravenously administered 0.5 mg/kg of 5′-CleanCap-5MeC-fLuciferase mRNA (TriLink Biotech) formulated with the LNP compositions shown in Table 4. One group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 6.

TABLE 6

| | Mean Liver Luciferase Flux |
|---|---|
| LNP ID | (p/s) |
| 3 | 2.8 × 10⁹ |
| 9 | 8.3 × 10¹⁰ |
| 11 | 1.4 × 10⁹ |
| 13 | 3.3 × 10¹⁰ |
| 24 | 5 × 10⁹ |
| 28 | 2.7 × 10¹⁰ |
| 29 | 9.1 × 10⁹ |
| 0 | 1.3 × 10⁹ |

As shown in Table 6, LNP compositions 3, 9, 11, 13, 24, 28 and 29 were capable of delivering mRNA in vivo, predominantly to cells in the liver, and subsequent expression of the encoded protein. Moreover, administration of LNP compositions 3, 9, 11, 13, 24, 28 and 29 resulted greatly improved liver luciferase signal as compared to the administration of LNP composition 0. LNP composition 0 is the most similar LNP composition to the LNP composition used in the art (see Tanaka et al. Advanced Functional Materials (2020) Vol: 30, 34). Accordingly, the LNP compositions of the present disclosure exhibit superior gene delivery activity as compared to standard LNP compositions used in the art.

In addition, the body weight of mice treated with the LNP compositions of Table 4a was assessed prior to intravenous administration and twenty-four hours post-administration and the body weights at baseline and post-treatment were compared. The average percentage of body weight change for each group of mice treated with each LNP composition of Table 7 is shown in Table 7.

TABLE 7

| | % Body Weight |
|---|---|
| LNP ID | Change 24 hr |
| 3 | +5.9 |
| 9 | +4.6 |
| 11 | +0.25 |
| 13 | +3.6 |
| 24 | +1.1 |
| 28 | −3.15 |
| 29 | +0.6 |

As shown in Table 7, the LNP compositions of the present disclosure were well tolerated with most treated mice retaining original body weights or even slightly gaining weight.

### Example 3—In Vivo LNP-Delivery of mRNA to Liver

A. Delivery sPB mRNA to Liver Cells

The following is a non-limiting example demonstrating that the compositions of the present disclosure can be used to deliver mRNA to liver cells, including hepatocytes, in vivo.

In this example, 5MeC-mRNA molecules comprising a sequence encoding an HA-tagged SPB protein were encapsulated in lipid nanoparticles of the present disclosure comprising about 28% of Coatsome SS-OP by moles, about 60% of cholesterol by moles, about 10% of DOPE by moles, and about 2% of DMG-PEG2000 by moles. The ratio of lipid to nucleic acid in the nanoparticles was about 100:1 (weight/weight) and the total lipid of 10 mM. The mRNA molecules were further capped using CleanCap®. As a negative staining control, mRNA comprising a sequence encoding a non-HA tagged sPB protein was used.

The lipid nanoparticles comprising the mRNA were administered to adult female BALB/C mice. The nanoparticles were administered as a single dose intravenously at an amount of 1 mg/kg. The mice were humanely euthanized at four hours after treatment and the livers of the mice were processed, e.g., blood was removed from the liver by flushing ˜10 mL of HBSS+2.5 mM EDTA through the portal vein, and analyzed using immunostaining for the HA tag as well as ELISA and Western Blot.

HA staining was observed in hepatocytes throughout the entire liver of the treated mice, with approximately 62% of all liver cells from one mouse and 66% of all liver cells from another mouse testing positive for sPB expression. Furthermore, in vivo expression of sPB was detected uniformly throughput each of the main liver lobes, the medial and left and right lateral lobes. Thus, the nanoparticle compositions of the present disclosure effectively deliver mRNA to hepatocytes throughout the entire liver in vivo, and that the delivered mRNA is subsequently translated into protein.

B. Dose-Dependent LNP Delivery of mRNA to Liver Cells and Tolerability

The following is a non-limiting example demonstrating that the lipid nanoparticle compositions of the present disclosure can be used to deliver mRNA to liver cells in vivo and expression of encoded proteins over a wide dose range with good tolerability.

Adult female BALB/C mice (n=3/group) were intravenously administered 0.5, 1.0, 2.0 or 3.0 mg/kg mRNA molecules comprising a sequence encoding an HA-tagged sPB protein formulated within LNP compositions prepared according to Example 1. One group of mice was left untreated as a negative control.

One group of mice were sacrificed at four hours after treatment and the livers of the mice were analyzed using immunostaining for the HA tag as well as ELISA and Western Blot. The results are shown in Table 8.

TABLE 8

| Dose (mg/kg) | HA-Tag sPB Protein (ng) |
|---|---|
| 0.5 | 0.8 |
| 1.0 | 1.5 |
| 2.0 | 1.9 |
| 3.0 | 2.4 |

As shown in Table 8, a linear dose response was observed for mice treated with a single dose of 0.5-3 mg/kg of HA-tagged sPB mRNA.

In another experiment, the duration of HA-tagged sPB protein expression was measured over time. Adult female BALB/C mice (n=3/group) were intravenously administered 0.5, 1.0, or 3.0 mg/kg mRNA molecules comprising a sequence encoding an HA-tagged sPB protein formulated within LNP composition 9 prepared according to Example 2. One group of mice was left untreated as a negative control.

One group of mice at each concentration was sacrificed at four hours, one group of mice at each concentration was sacrificed at twenty-four hours and one group of mice at each concentration was sacrificed seven days after treatment and the livers of the mice were analyzed using immunostaining for the HA tag as well as ELISA. The results are shown in Table 9.

TABLE 9

| Dose (mg/kg) | Time (hr) | HA-Tag sPB Protein (ng) |
|---|---|---|
| 0.5 | 4 | 0.8 |
| 1.0 | 4 | 1.5 |
| 3.0 | 4 | 2.4 |
| 0.5 | 24 | 0.2 |
| 1.0 | 24 | 0.8 |
| 3.0 | 24 | 1.2 |
| 0.5 | 168 | 0.1 |
| 1.0 | 168 | 0.1 |
| 3.0 | 168 | 0.2 |

As shown in Table 9, the expression of HA-tagged sPB protein decreased over time at each concentration tested with sPB expression declining to near baseline levels by Day 7.

In addition, the levels of three liver enzymes present in serum was evaluated at 24 hours and 7 days after LNP administration for each of the tested concentrations as a measure of potential hepatotoxicity. Briefly, blood was drawn at 24 hours and at 7 days and each sample was allowed to clot for 20 minutes and subject to centrifugation at 13K rpms for 3 minutes to remove undesired cells and debris. The samples were placed on wet ice for transport, and store at −80° C. until analyzed. Enzyme levels were determined using standardized assays (Idexx).

The levels of the liver enzymes aspartate transaminase (AST), alanine transaminase (ALT) and alkaline phosphatase (ALP) at 24 hours and at 7 days are shown in Tables 10 a-c, respectively.

TABLE 10a
AST Levels

| LNP Dose (mg/kg) | Time (hrs) | AST Amount (Units/L) |
|---|---|---|
| 0 | 24 | 49 |
| 0.5 | 24 | 54 |
| 1.0 | 24 | 66 |
| 2.0 | 24 | 78 |
| 3.0 | 24 | 87 |
| 0 | 168 | 93 |
| 0.5 | 168 | 58 |
| 1.0 | 168 | 85 |
| 2.0 | 168 | 69 |
| 3.0 | 168 | 54 |

TABLE 10b
ALT Levels

| LNP Dose (mg/kg) | Time (hrs) | ALT Amount (Units/L) |
|---|---|---|
| 0 | 24 | 29 |
| 0.5 | 24 | 30 |
| 1.0 | 24 | 36 |
| 2.0 | 24 | 46 |
| 3.0 | 24 | 59 |
| 0 | 168 | 44 |
| 0.5 | 168 | 29 |
| 1.0 | 168 | 44 |
| 2.0 | 168 | 29 |
| 3.0 | 168 | 35 |

TABLE 10c
ALP Levels

| LNP Dose (mg/kg) | Time (hrs) | ALP Amount (Units/L) |
|---|---|---|
| 0 | 24 | 81 |
| 0.5 | 24 | 88 |
| 1.0 | 24 | 83 |
| 2.0 | 24 | 80 |
| 3.0 | 24 | 77 |
| 0 | 168 | 94 |
| 0.5 | 168 | 83 |
| 1.0 | 168 | 77 |
| 2.0 | 168 | 78 |
| 3.0 | 168 | 72 |

As shown in Tables 10a-c, the in vivo administration of the LNP compositions of the present disclosure resulted in a very slight (<2×) dose-dependent increase in AST and ALT levels in serum at 24 hours; however, all three enzyme levels resolved back to baseline by Day 7 demonstrating that the magnitude of liver enzyme elevation was low.

In addition to serum liver enzymes, the levels of three proinflammatory cytokines present in serum was evaluated at 4 hours after LNP administration for each of the tested concentrations. Briefly, serum samples were prepared as described for liver enzyme analysis and the serum concentration of each cytokine was determined using commercially available ELISA kits (e.g., R&D Systems Quantikine ELISA kits). The levels of the proinflammatory cytokines interleukin-6 (IL-6), interferon gamma (INF-G) and tumor necrosis factor alpha (TNF-a) at 4 hours are shown in Table 11 a-c, respectively.

TABLE 11a
IL-6 Levels

| LNP Dose (mg/kg) | Time (hrs) | Amount (pg/mL) |
|---|---|---|
| 0 | 24 | 0 |
| 0.5 | 24 | 0.1 |
| 1.0 | 24 | 1.0 |
| 2.0 | 24 | 74.8 |
| 3.0 | 24 | 238.7 |

TABLE 11b
INF-G Levels

| LNP Dose (mg/kg) | Time (hrs) | Amount (pg/mL) |
|---|---|---|
| 0 | 24 | 0 |
| 0.5 | 24 | 5.6 |
| 1.0 | 24 | 17.1 |
| 2.0 | 24 | 17.1 |
| 3.0 | 24 | 33.6 |

TABLE 11c
TNF-a Levels

| LNP Dose (mg/kg) | Time (hrs) | Amount (pg/mL) |
|---|---|---|
| 0 | 24 | 0 |
| 0.5 | 24 | 1.3 |
| 1.0 | 24 | 7.4 |
| 2.0 | 24 | 8.7 |
| 3.0 | 24 | 15.4 |

As shown in Tables 11a-c, the in vivo administration of the LNP compositions of the present disclosure resulted in a dose-dependent increase in serum pro-inflammatory cytokines though the magnitude of the response was modest in view of other non-bioreducible ionizable cationic lipids.

In yet another experiment, the LNP composition 9 from Example 2 was compared to a LNP particle comprising the non-bioreducible ionizable cationic lipid, C12-200. Adult female BALB/C mice (n=3/group) were intravenously administered 0.5, 1.0, or 3.0 mg/kg mRNA molecules comprising a sequence encoding an HA-tagged sPB protein formulated within LNP compositions. One group of mice was left untreated as a negative control. The percentage of SB positive hepatocytes, ALT liver enzyme measurements and IL-6 cytokine release measurements were compared between the treated animals for each group and the values for each at the 1 mg/kg dose (MTD dose for C12-200 LNP composition) are reported in Table 12.

TABLE 12

| LNP | % + | ALT Concentration | IL-6 Concentration |
|---|---|---|---|
| Composition | Hepatocytes | (U/L) | (pg/ml) |
| 9 | 64.4 | 28.33 | 0.97 |
| C12-200 | 72.5 | 241.33 | 928.3 |

As shown in Table 12, LNP composition 9 exhibited similar efficacy at delivering SB mRNA to hepatocytes while simultaneously exhibiting a significantly reduced toxicity profile as compared to the C12-200 LNP composition as denoted by the reduced IL-6 cytokine release and reduced ALT liver enzyme compared to C12-200 LNP.

C. LNP Compositions of Present Disclosure Deliver RNA with High Specificity to the Liver In Vivo.

In a third experiment, one group of adult female BALB/C mice (n=3/group) was intravenously administered 1 mg/kg HA-tagged sPB mRNA formulated within nanoparticle compositions prepared according to Example 1, and a second group was left untreated as a control. After four hours post-administration, the mice from each group were humanely euthanized and four tissue types were collected: liver, spleen, lung, and kidney.

Collected tissues were processed, e.g., blood was removed from the liver by flushing ˜10 mL of HBSS+2.5 mM EDTA through the portal vein. Protein extraction buffer was prepared by adding protease inhibitor (HALT, ThermoFisher #78439) to T-PER (ThermoFisher #78510) at a 1:50 (v:v) ratio. The protein extraction buffer was stored at room temperature for up to 1 hour. Tissue samples were added to the protein extraction buffer at a ratio of 9 mL per 1 g of tissue in an RNAse-free Eppendorf tube (Invitrogen #Am12425). One tungsten carbide bead (3 mm; Qiagen #69997) was added to the solution, and the tube placed into a pre-cooled adaptor block in tissue disruptor (Qiagen TissueLyzer II). The sample was lysed by shaking for 5 minutes at 25 Hz. The sample was clarified by centrifugation for 10 minutes at 14.8k at 4 deg C. The supernatant was collected and the pellet discarded.

Total protein was measured by the BCA assay using a commercially available kit (BCA Assay Kit, Pierce #23225). Clarified liver lysate was diluted 200×, incubated at 37 deg C. for 30 minutes, and absorbance read at 562 nm. The results are shown in Table 13.

TABLE 13

| Tissue | HA-Tag sPB Protein (ng) |
|---|---|
| Liver | 2.4 |
| Spleen | 0.1 |
| Lung | 0 |
| Kidney | 0 |

As shown in Table 13, HA tagged-sPB expression was detected almost exclusively in the liver of treated animals with minimal expression in the spleen and no detectable expression in the lung or kidney demonstrating the ability of LNP composition of the present disclosure to preferentially deliver mRNA in vivo to liver and the subsequent expression of the encoded polypeptide in liver hepatocytes.

The result presented in Example 3 demonstrate that the LNP compositions of the present disclosure are capable of effectively delivering mRNA to the liver in vivo which is then expressed within the cells of the liver, and that protein expression can be controlled by administered dose over a wide range. Moreover, the LNP compositions of the present disclosure are well-tolerated and exhibit low levels of toxicity.

### Example 4—Preparation of LNP Compositions of the Present Disclosure Comprising DNA

The following is a nonlimiting example that demonstrates that a DNA nanoplasmid (a circular DNA) can be incorporated in LNP compositions of the present disclosure.

LNP compositions of the present disclosure comprising DNA, a nanoplasmid encoding a piggyBac transposon, wherein the piggyBac transposon comprised luciferase under control of the CMV promoter (herein referred to as the pB-nanofluc2), and Coatsome SS-OP, the phospholipid DOPE, the structural lipid cholesterol (Chol) and 1,2-dimyristoyl-sn-glycerol methoxypolyethylene glycol (DMG-PEG2000) in the following percentages in Table 14:

TABLE 14

| | | | | | Lipid | |
|---|---|---|---|---|---|---|
| LNP | Coatsome | | | DMG- | Blend | Lipid:DNA |
| ID | SS—OP | DOPE | Cholesterol | PEG2000 | (mM) | (w/w) |
| D1 | 22.71 | 20.83 | 55.21 | 1.25 | 15 | 100:1 |
| D2 | 50 | 10 | 38.6 | 1.4 | 10 | 200:1 |
| D3 | 34.69 | 24.69 | 39.74 | 1.25 | 10 | 50:1 |
| D4 | 50 | 29.5 | 20 | 0.5 | 10 | 100:1 |
| D5 | 50 | 28.7 | 20 | 1.3 | 10 | 50:1 |

Adult BALB/C mice were administered 1.0 mg/kg of total DNA (n=4) of each of the LNP compositions D1-D5 listed in Table 14. The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 15.

TABLE 15

| | Mean Liver Luciferase |
|---|---|
| LNP ID | Flux (p/s) |
| D1 | 2.5e7 |
| D2 | 2.0e7 |
| D3 | 1.5e7 |
| D4 | 1.0e7 |
| D5 | 1.8e7 |

As shown in Table 15, all of the LNP compositions were capable of delivering DNA to liver cells and express the encoded transgene in liver cells.

### Example 5—LNPs of the Present Disclosure for the Treatment of Hemophilia

The following is a non-limiting example demonstrating the compositions and methods of the present disclosure can be used to in the treatment of hemophilia, and more specifically hemophilia A.

Adult Mice

Female, adult (8-9 weeks), wild-type BALB/c were first administered 0.5 mg/kg FVIII transposon LNPs on Day 0 of the experiment and then administered 3.0 mg/kg HA-SPB LNPs on Day 7. Total injection volumes on both Day 0 and Day 7 were 200 μL per mouse. Upon dosing, mice were restrained and injected intravenously through the tail vein using a 29 gauge insulin syringe.

The FVIII transposon LNPs were C12-200-containing LNPs comprising nanoplasmid DNA comprising a transposon, wherein the transposon comprised an expression cassette comprising, a first piggyBac inverted terminal repeat (ITR), followed by a first insulator sequence, followed by a Transthyretin (TTR) enhance/promoter and minute virus of mice (MVM) intron sequence, followed by a codon optimized nucleic acid sequence encoding for human Factor VIII (FVIII) lacking the B-domain (hereafter referred to as FVIII-BDD), followed by an SV40 polyA sequence, followed by a second insulator sequence, followed by a second piggyBac ITR. The sequence of the transposon is put forth in SEQ ID NO: 35. The FVIII transposon LNPs comprised C12-200, DOPE, Cholesterol and DMG-PEG2000 at a molar ratio of 0.35:0.2:0.4184:0.0316 and had a lipid:DNA ratio of 80:1 (w/w).

The HA-SPB LNPs were ssPalmO-Ph-P4C2-containing LNPs of the present disclosure comprising mRNA encoding active SPB. All cytidine residues in the mRNA were 5-methylcytidine (5-MeC). The HA-SPB LNPs comprised ssPalmO-Ph-P4C2, DOPE, Cholesterol and DMG-PEG2000 at a molar ratio of 28:10:60:2 and had a lipid:RNA ratio 100:1 (w/w).

On Day 6 and 13 after the first Day 0 injection, plasma was collected by retro-orbital bleeding. Briefly, plain uncoated pasteur pipets were used to disrupt the retrobulbar venous sinus and whole blood was collected and mixed with 3.2% buffered sodium citrate at a 9:1 ratio by volume. This mixture was centrifuged at 15,000 g for 15 minutes at 22° C. and plasma supernatant was collected and stored at −80° C. Human FVIII protein levels in the samples were analyzed using the Affinity Biologicals™, Inc. VisuLize™ Factor FVIII Antigen Plus Kit per manufacturer's instructions.

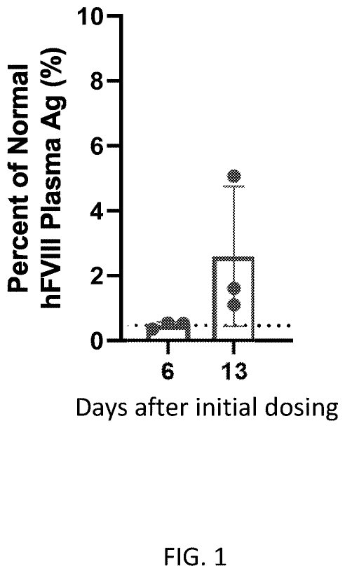

The results of this analysis are shown in FIG. 1. FIG. 1 shows that Human FVIII protein levels were observed in the samples at a level corresponding to between 1-5% of normal human FVIII on Day 13 following administration of the HA-SPB LNPs on Day 6.

The results presented in this example demonstrate that the LNPs of the present disclosure can be used to drive high levels of FVIII expression in vivo, even in adult mice, thereby demonstrating that the composition and methods of the present disclosure can be used to treat hemophilia A.

### Example 6—LNPs of the Present Disclosure for the Treatment of Hemophilia

The following is a non-limiting example demonstrating the compositions and methods of the present disclosure can be used to in the treatment of hemophilia, and more specifically hemophilia B.

Juvenile Mice

Three-week old juvenile C57BL/6 mice were either left untreated, or were administered one of the following two treatments:

Treatment #1: Factor IX transposon AAV viral vector particles

Treatment #2: Factor IX transposon AAV viral vector particles in combination with SPB LNPs.

The Factor IX transposon AAV viral vector particles were AAV viral vector particles comprising a piggyBac transposon, wherein the piggyBac transposon comprised a nucleic acid encoding for a human Factor IX polypeptide with the R338L mutation.

The SPB LNPs were ssPalmO-Ph-P4C2-containing LNPs of the present disclosure comprising mRNA encoding active SPB. The SPB LNPs comprised ssPalmO-Ph-P4C2, DOPE, Cholesterol and DMG-PEG2000 at a molar ratio of 28:10:60:2 and had a lipid:RNA ratio 100:1 (w/w).

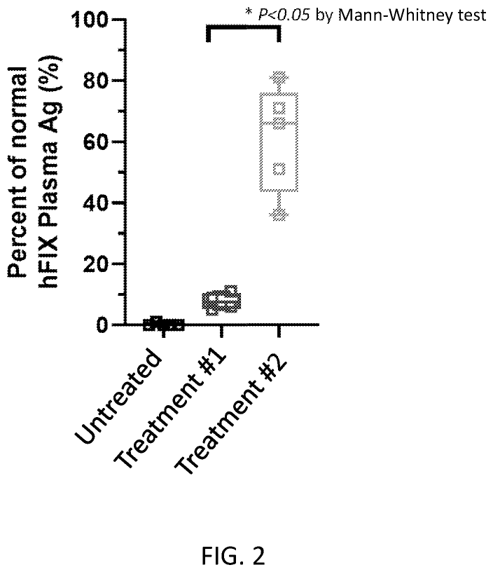

Three weeks following administration of the treatments, ELISA experiments were performed to determine the amount of human Factor IX polypeptide in the plasma of the mice. The results of these ELISA experiments are shown in FIG. 2. FIG. 2 shows that human Factor IX protein levels were observed in the samples at a level corresponding to between about 40-85% of normal human Factor IX following administration of Treatment #2 comprising LNPs of the present disclosure.

The results presented in this example demonstrate that the LNPs of the present disclosure can be used to drive high levels of Factor IX expression in vivo, thereby demonstrating that the composition and methods of the present disclosure can be used to treat hemophilia B. More specifically, the LNPs of the present disclosure can be used to drive levels of Factor IX expression in vivo that is in the range of Factor IX levels observed in healthy individuals.

### Example 7—Preparation of LNPs of Present Disclosure Comprising mRNA, In Vivo Screening and Stability Tests

A. Preparation

The following is a nonlimiting example that provides exemplary methods for formulating a plurality of multi-component LNP compositions comprising a bioreducible ionizable cationic lipid and mRNA.

To formulate the LNPs, various percentages of the bioreducible ionizable cationic lipid ssPalmO-Ph-P4C2, the phospholipid DOPE, the structural lipid cholesterol (Chol) and 1,2-dimyristoyl-sn-glycerol methoxypolyethylene glycol (DMG-PEG2000; Avanti Polar Lipids, Alabaster, Alabama, USA) were combined to prepare LNP compositions.

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temp and then placed on a hot plate maintained at a temperature range of 50-55° C. Subsequently, the hot lipid stock solutions were combined to yield desired final mol percentages. A subset of the LNP compositions are shown in Tables 16:

TABLE 16

| | | | | | Total | |
|---|---|---|---|---|---|---|
| LNP | ssPalmO—Ph—P4C2 | DOPE | Chol | PEG-DMG | Lipid | Lipid:RNA |
| ID | (% moles) | (% moles) | (% moles) | (% moles) | (mM) | (w/w) |
| 2.1 | 19.00% | 16.00% | 60.00% | 5.00% | 15 | 40 |
| 2.2 | 24.00% | 17.00% | 55.00% | 4.00% | 23 | 96 |
| 2.3 | 26.93% | 18.63% | 50.67% | 3.77% | 25 | 100 |
| 2.4 | 20.93% | 21.79% | 52.64% | 4.64% | 15 | 77 |
| 2.5 | 24.67% | 11.68% | 58.79% | 4.86% | 24 | 70 |
| 2.6 | 54.00% | 10.00% | 35.00% | 1.00% | 25 | 100 |
| 2.7 | 26.02% | 20.44% | 50.00% | 3.54% | 25 | 49 |
| 2.8 | 21.00% | 19.00% | 55.00% | 5.00% | 25 | 40 |
| 2.9 | 28.00% | 10.00% | 60.00% | 2.00% | 10 | 100 |
| 2.10 | 27.84% | 13.46% | 56.25% | 2.45% | 19 | 88 |
| 2.11 | 27.90% | 18.59% | 51.51% | 2.00% | 15 | 100 |
| 2.12 | 26.28% | 22.65% | 50.01% | 1.06% | 17 | 40 |
| 2.13 | 21.00% | 17.00% | 60.00% | 2.00% | 15 | 40 |
| 2.14 | 60.00% | 11.60% | 26.40% | 2.00% | 15 | 70 |
| 2.15 | 22.00% | 21.00% | 55.00% | 2.00% | 15 | 40 |
| 2.16 | 30.37% | 30.36% | 37.27% | 2.00% | 15 | 100 |

A 1 mg/ml solution of the 5meC 5′-CleanCap-5MeC-SPB-HA mRNA (prepared as in Specific Example 1) to be incorporated into the LNPs was added to 150 mM sodium acetate buffer (pH 5.2) or maleate (pH 5.0) to form a stock solution and kept on ice. The mRNA comprised a nucleic acid sequence that encoded an HA-tagged SBP polypeptide. The lipid phase was mixed with the aqueous mRNA phase inside a microfluidic chip using a NanoAssemblr® instrument (Precision Nanosystems, Vancouver, BC, Canada) according to the manufacturer's instructions to form LNP compositions comprising encapsulated mRNAs. Nanoassemblr® process parameters, including the buffer used, the buffer concentration and the flow rate, for mRNA encapsulation for each of the LNP compositions are shown in the Table 17:

TABLE 17

| | | Buffer | Flow Rate |
|---|---|---|---|
| LNP ID | Buffer | Concentration (mM) | (ml/min) |
| 2.1 | Sodium acetate (pH 5.2) | 10 | 20 |
| 2.2 | Sodium acetate (pH 5.2) | 150 | 20 |
| 2.3 | Sodium acetate (pH 5.2) | 10 | 20 |
| 2.4 | Sodium acetate (pH 5.2) | 100 | 20 |
| 2.5 | Sodium acetate (pH 5.2) | 50 | 20 |
| 2.6 | Maleate (pH 5) | 50 | 4 |
| 2.7 | Sodium acetate (pH 5.2) | 150 | 20 |
| 2.8 | Sodium acetate (pH 5.2) | 10 | 20 |
| 2.9 | Sodium acetate (pH 5.2) | 150 | 20 |
| 2.10 | Sodium acetate (pH 5.2) | 100 | 20 |
| 2.11 | Maleate (pH 5) | 100 | 12 |
| 2.12 | Sodium acetate (pH 5.2) | 150 | 20 |
| 2.13 | Sodium acetate (pH 5.2) | 10 | 20 |
| 2.14 | Maleate (pH 5) | 150 | 4 |
| 2.15 | Sodium acetate (pH 5.2) | 10 | 20 |
| 2.16 | Maleate (ph 5) | 150 | 4 |

The resultant mRNA LNP compositions were then transferred to a Repligen Float-A-Lyzer dialysis device—having a molecular weight cut off (MWCO) of 8-10 kDa (Spectrum Chemical Mfg. Corp, CA, USA) and processed by dialysis against phosphate buffered saline (PBS) (dialysate:dialysis buffer volume at least 1:200 v/v), pH 7.4 or 6.5 overnight at 4° C. (or alternatively room temperature for at least 4 hours), to remove the 25% ethanol and achieve a complete buffer exchange. In some experiments the LNPs were further concentrated in an Amicon® Ultra-4 centrifugal filter unit, MWCO-100 kDa or 50 kDa (Millipore Sigma, USA) spun at 4000×g in an ultracentrifuge. The mRNA LNPs were then stored at 4° C. until further use.

B. In Vivo Screening

Adult female BALB/C mice (n=2/group) were intravenously administered 1.0 mg/kg of the LNP compositions comprising HA-SBP mRNA shown in Table 16. One group of mice was treated with vehicle (PBS, Thermo Fisher Scientific, USA) as a negative control.

Mice were euthanized 4 hours after dosing and their livers were collected. The expression of HA-SBP was assessed by ELISA. The results of this analysis are shown in Table 18:

TABLE 18

| | Mouse 1 Liver | Mouse 2 Liver | |
|---|---|---|---|
| LNP ID | Expression | Expression | Mean |
| 2.1 | 0.03 | 0.04 | 0.04 |
| 2.2 | 0.01 | 0 | 0.01 |
| 2.3 | 0.01 | 0.01 | 0.01 |
| 2.4 | 0 | 0.1 | 0.05 |
| 2.5 | 0 | 0 | 0 |
| 2.6 | 1.21 | 0.76 | 0.98 |
| 2.7 | 0.02 | 0.08 | 0.05 |
| 2.8 | 0.04 | 0 | 0.02 |
| 2.9 | 0.01 | NA | 0.01 |
| 2.10 | 1.06 | 0.47 | 0.76 |
| 2.11 | 1.04 | 1.49 | 1.27 |
| 2.12 | 0.17 | 0.16 | 0.16 |
| 2.13 | 0.06 | 0.08 | 0.07 |
| 2.14 | 0.76 | 0.62 | 0.69 |
| 2.15 | 0.21 | 0.12 | 0.17 |
| 2.16 | 1.09 | 1.04 | 1.07 |
| PBS (vehicle) | 0 | 0 | 0 |

As show in Table 18, LNP ID 2.6, LNP ID 2.10, LNP ID 2.11, LNP 2.14 and LNP 2.16 were capable of delivering mRNA in vivo, specifically to cells in the liver, and there was subsequent expression of the encoded protein.

C. Stability Tests

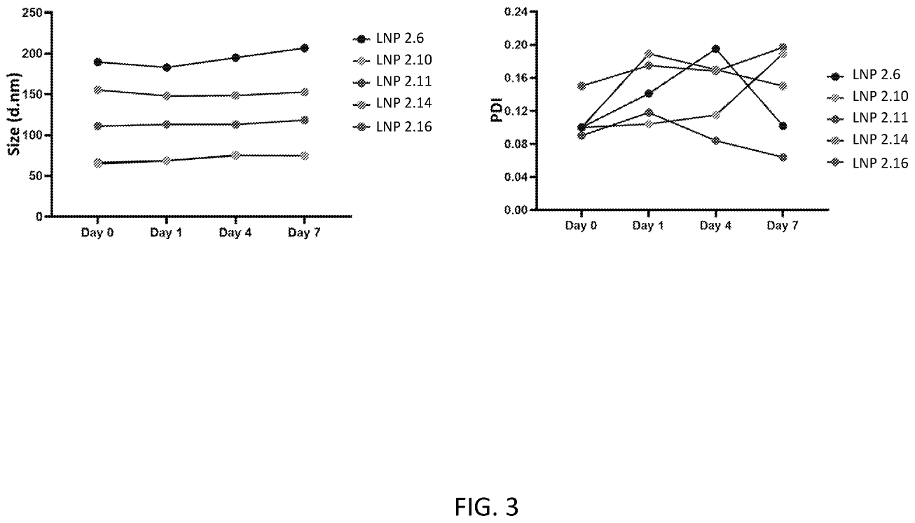

Aliquots of LNP ID 2.6, LNP ID 2.10, LNP ID 2.11, LNP 2.14 and LNP 2.16 were each stored at 0.1 mg/mL at 4° C. for 7 days to assess storage stability of the LNP compositions. The mean diameter and polydispersity index (PDI) for each of the LNP compositions was analyzed at the beginning of the 7-day incubation (Day 0), and on Day 1, Day 4 and Day 7 of the incubation. The results of this analysis are shown in FIG. 3. As shown in FIG. 3, the size and PDI for each of LNP ID 2.6, LNP ID 2.10, LNP ID 2.11, LNP 2.14 and LNP 2.16 were stable over the course of the 7 day incubation, indicating that these compositions show enhanced storage stability, which is advantageous in clinical and commercial settings.

Taken together, the results described in this example demonstrate that the LNP compositions of the present disclosure can effectively deliver mRNA to cell in vivo, including liver cells, and that these compositions are stable at standard storage temperatures over extended time periods.

### Example 8—Preparation of LNPs of Present Disclosure Comprising mRNA and In Vivo Screening

A. Preparation

The following is a nonlimiting example that provides exemplary methods for formulating a plurality of multi-component LNP compositions comprising a bioreducible ionizable cationic lipid and mRNA.

To formulate the LNPs, various percentages of the bioreducible ionizable cationic lipid ssPalmO-Ph-P4C2, the phospholipid (DOPE, DSPC, or DOPC), the structural lipid cholesterol (Chol) and 1,2-dimyristoyl-sn-glycerol methoxypolyethylene glycol (DMG-PEG2000; Avanti Polar Lipids, Alabaster, Alabama, USA) were combined to prepare LNP compositions.

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temp and then placed on a hot plate maintained at a temperature range of 50-55° C. Subsequently, the hot lipid stock solutions were combined to yield desired final mol percentages. A subset of the LNP compositions is shown in Table 19.

TABLE 19

| | | | | | Total | |
|---|---|---|---|---|---|---|
| LNP | ssPalmO—Ph—P4C2 | Phospholipid | Chol | PEG-DMG | Lipid | Lipid:RNA |
| ID | (% moles) | (% moles) | (% moles) | (% moles) | (mM) | (w/w) |
| 3.1 | 54 | 10% DOPE | 35 | 1 | 25 | 100 |
| 3.2 | 54 | 10% DOPC | 35 | 1 | 25 | 100 |
| 3.3 | 54 | 10% DOPE | 35 | 1 | 25 | 60 |
| 3.4 | 54 | 10% DOPC | 35 | 1 | 25 | 60 |
| 3.5 | 49.4 | 5% DOPC | 44 | 1.6 | 25 | 60 |
| 3.6 | 60 | 5% DSPC | 32.8 | 2.2 | 25 | 60 |
| 3.7 | 60 | 5% DOPC | 34 | 1 | 25 | 40 |
| 3.8 | 41.8 | 5% DSPC | 52.2 | 1 | 25 | 60 |
| 3.9 | 54 | 10% DSPC | 35 | 1 | 25 | 60 |
| 3.10 | 54 | 10% DSPC | 35 | 1 | 25 | 100 |

A 1 mg/ml solution of the 5′-CleanCap-5MeC-fLuciferase mRNA (TriLink Biotech) to be incorporated into the LNPs was added to 150 mM sodium acetate buffer (pH 5.2) to form a stock solution and kept on ice. The lipid phase was mixed with the aqueous mRNA phase inside a microfluidic chip using a NanoAssemblr® instrument (Precision Nanosystems, Vancouver, BC, Canada) according to the manufacturer's instructions to form LNP compositions comprising encapsulated mRNAs. Nanoassemblr® process parameters for mRNA encapsulation are shown in the Table 20.

TABLE 20

| Total flow rate | Lipid phase: aqueous |
|---|---|
| (ml/min) | (RNA) phase (v/v) |
| 20 | 1:3 |

The resultant mRNA LNP compositions were then transferred to a Repligen Float-A-Lyzer dialysis device—having a molecular weight cut off (MWCO) of 8-10 kDa (Spectrum Chemical Mfg. Corp, CA, USA) and processed by dialysis against 25 mM sodium acetate (dialysate:dialysis buffer volume at least 1:200 v/v), pH 5.5 overnight at 4° C. (or alternatively room temperature for at least 4 hours), to remove the 25% ethanol and achieve a complete buffer exchange. In some experiments the LNPs were further concentrated in an Amicon® Ultra-4 centrifugal filter unit, MWCO-30 kDa (Millipore Sigma, USA) spun at 4100×g in an ultracentrifuge. Sucrose was added to a final concentration of 5% (w/v) to the mRNA LNPs which were then stored at 4° C. or frozen at −80° C. until further use.

The average particle size diameter of the LNPs ranged from approximately 84-121 nm.

B. In Vivo Screening

Adult female BALB/C mice (n=2/group) were intravenously administered 0.5 mg/kg of 5′-CleanCap-5MeC-fLuciferase mRNA (TriLink Biotech) formulated with the LNP compositions shown in Table 19. One group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 21.

TABLE 21

| | Mean Liver Luciferase |
|---|---|
| LNP ID | Flux (p/s) |
| 3.1 | 5.04E+10 |
| 3.2 | 1.88E+10 |
| 3.3 | 1.64E+10 |
| 3.4 | 1.36E+10 |
| 3.5 | 6.72E+09 |
| 3.6 | 3.84E+09 |
| 3.7 | 3.71E+09 |
| 3.8 | 3.70E+09 |
| 3.9 | 3.37E+09 |
| 3.10 | 2.56E+09 |

As shown in Table 21, LNP compositions 3.1-3.10 were capable of delivering mRNA in vivo, predominantly to cells in the liver, and subsequent expression of the encoded protein.

### Example 9—Preparation of LNPs of Present Disclosure Comprising mRNA and In Vivo Screening

A. Preparation

The following is a nonlimiting example that provides exemplary methods for formulating a plurality of multi-component LNP compositions comprising a bioreducible ionizable cationic lipid and mRNA.

To formulate the LNPs, various percentages of the bioreducible ionizable cationic lipid ssPalmO-Ph-P4C2, the phospholipid (DOPE, DSPC, or DOPC), the structural lipid cholesterol (Chol) and 1,2-dimyristoyl-sn-glycerol methoxypolyethylene glycol (DMG-PEG2000; Avanti Polar Lipids, Alabaster, Alabama, USA) were combined to prepare LNP compositions.

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temp and then placed on a hot plate maintained at a temperature range of 50-55° C. Subsequently, the hot lipid stock solutions were combined to yield desired final mol percentages. A subset of the LNP compositions is shown in Table 22.

TABLE 22

| | | | | | Total | |
|---|---|---|---|---|---|---|
| LNP | ssPalmO—Ph—P4C2 | Phospholipid | Chol | PEG-DMG | Lipid | Lipid:RNA |
| ID | (% moles) | (% moles) | (% moles) | (% moles) | (mM) | (w/w) |
| 4.1 | 54 | 10% DOPE | 35 | 1 | 25 | 100 |
| 4.2 | 54 | 10% DOPC | 35 | 1 | 25 | 100 |
| 4.3 | 54 | 10% DSPC | 35 | 1 | 25 | 100 |
| 4.4 | 59 | 5% DOPC | 35 | 1 | 25 | 100 |
| 4.5 | 59 | 5% DSPC | 35 | 1 | 25 | 100 |
| 4.6 | 54 | 5% DOPC | 40 | 1 | 25 | 100 |
| 4.7 | 54 | 5% DSPC | 40 | 1 | 25 | 100 |
| 4.8 | 56.5 | 5% DOPC | 37.5 | 1 | 25 | 100 |
| 4.9 | 56.5 | 5% DSPC | 37.5 | 1 | 25 | 100 |
| 4.10 | 54 | 10% DOPE | 35 | 1 | 25 | 75 |
| 4.11 | 54 | 10% DOPC | 35 | 1 | 25 | 75 |
| 4.12 | 59 | 5% DOPC | 35 | 1 | 25 | 75 |

A 1 mg/ml solution of the 5′-CleanCap-5MeC-fLuciferase mRNA (TriLink Biotech) to be incorporated into the LNPs was added to 150 mM sodium acetate buffer (pH 5.2) to form a stock solution and kept on ice. The lipid phase was mixed with the aqueous mRNA phase inside a microfluidic chip using a NanoAssemblr® instrument (Precision Nanosystems, Vancouver, BC, Canada) according to the manufacturer's instructions to form LNP compositions comprising encapsulated mRNAs. Nanoassemblr process parameters for mRNA encapsulation are shown in the Table 23.

TABLE 23

| Total flow rate | Lipid phase:aqueous |
|---|---|
| (ml/min) | (RNA) phase (v/v) |
| 20 | 1:3 |

The resultant mRNA LNP compositions were then transferred to a Repligen Float-A-Lyzer dialysis device—having a molecular weight cut off (MWCO) of 8-10 kDa (Spectrum Chemical Mfg. Corp, CA, USA) and processed by dialysis against 25 mM sodium acetate (dialysate:dialysis buffer volume at least 1:200 v/v), pH 5.5 overnight at 4° C. (or alternatively room temperature for at least 4 hours), to remove the 25% ethanol and achieve a complete buffer exchange. In some experiments the LNPs were further concentrated in an Amicon® Ultra-4 centrifugal filter unit, MWCO-30 kDa (Millipore Sigma, USA) spun at ˜4100×g in an ultracentrifuge. Sucrose was added to a final concentration of 5% (w/v) to the mRNA LNPs which were then stored at 4° C. or frozen at −80° C. until further use.

The average particle size diameter of the LNPs ranged from approximately 80-103 nm.

B. In Vivo Screening

Adult female BALB/C mice (n=2/group) were intravenously administered 0.5 mg/kg of 5′-CleanCap-5MeC-fLuciferase mRNA (TriLink Biotech) formulated with a subset of the LNP compositions shown in Table 22. One group of mice was treated with vehicle (PBS, Thermo Fisher Scientific, USA) as a negative control.

In another experiment, adult female BALB/C mice (n=3-4/group) were intravenously administered 1 mg/kg of 5′-CleanCap-5MeC-fLuciferase mRNA (TriLink Biotech) formulated with a subset of the LNP compositions shown in Table 22. One group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results for the 0.5 mg/kg dose experiment are shown in Table 24 and the 1 mg/kg dose experiment are shown in Table 25.

TABLE 24

| | Mean Liver Luciferase |
|---|---|
| LNP ID | Flux (p/s) |
| 4.1 | 3.42E+10 |
| 4.2 | 3.33E+10 |
| 4.3 | 1.53E+10 |
| 4.4 | 3.58E+10 |
| 4.5 | 1.26E+10 |
| 4.6 | 2.17E+10 |
| 4.7 | 1.37E+10 |
| 4.8 | 2.82E+10 |
| 4.9 | 1.42E+10 |

TABLE 25

| | Mean Liver Luciferase |
|---|---|
| LNP ID | Flux (p/s) |
| 4.1 | 1.05E+11 |
| 4.10 | 1.27E+11 |
| 4.2 | 1.51E+11 |
| 4.11 | 7.51E+10 |
| 4.4 | 1.07E+11 |
| 4.12 | 1.05E+11 |

As shown in Tables 24 and 25, LNP compositions of the present disclosure were capable of delivering mRNA in vivo, predominantly to cells in the liver, and subsequent expression of the encoded protein.

In addition, the body weight of mice treated with a subset of the LNP compositions of Table 22 was assessed prior to intravenous administration and twenty-four hours post-administration and the body weights at baseline and post-treatment were compared. The average percentage of body weight change for each group of mice treated with each LNP composition of Table 22 is shown in Table 26.

TABLE 26

| | % Body Weight |
|---|---|
| LNP ID | Change 24 hr |
| 4.1 | −7.3 |
| 4.10 | −2.2 |
| 4.2 | +2.7 |
| 4.11 | −1.7 |
| 4.4 | −5.4 |
| 4.12 | −4.7 |

As shown in Table 26, the LNP compositions of the present disclosure were well tolerated with most treated mice retaining original body weights or even slightly gaining weight.

### Example 10—LNP Compositions of the Present Disclosure Reduce Immune Response and Toxicity

A. Immune Response to LNPs of the Present Disclosure

The following is a non-limiting example demonstrating that the in vitro administration of specific LNP compositions of the present disclosure resulted in a decrease in complement activation as measured by serum levels of C3a in human serum.

LNP compositions were prepared as described in Example 8 with the following mole percentages shown in Table 27. The LNP compositions encapsulated RNA molecules comprising a sequence encoding HA-tagged SPB.

TABLE 27

| | | | | | Total | |
|---|---|---|---|---|---|---|
| LNP | ssPalmO—Ph—P4C2 | Phospholipid | Chol | PEG-DMG | Lipid | Lipid:RNA |
| ID | (% moles) | (% moles) | (% moles) | (% moles) | (mM) | (w/w) |
| 5.0 | 54 | 10% DOPE | 35 | 1 | 25 | 100 |
| 5.2 | 54 | 10% DOPC | 35 | 1 | 25 | 100 |
| 5.3 | 54 | 10% DSPC | 35 | 1 | 25 | 100 |

Normal human serum (NHS) was thawed at 37° C. and 100 μL was aliquoted into a 1.5 mL centrifuge tube. NHS was then treated with 16 μL of an LNP composition at 0.1 mg/mL and incubated for 30 mins at 37° C. The reaction mixtures were then diluted 1:5000 and analyzed using a C3a ELISA kit (Quidel).

The levels of C3a in the samples treated with the LNP compositions of the present disclosure were compared to the level of C3a in the sample treated with an LNP composition comprising a benchmark phosphoethanolamine (PE)-based phospholipid and the values are reported in Table 28.

TABLE 28

| LNP | C3a Concentration |
|---|---|
| Composition | (ng/mL) |
| 5.0 | 10,566.7 |
| 5.2 | 4955.6 |
| 5.3 | 4633.4 |

As shown in Table 28, LNP compositions 5.2 and 5.3 of the present disclosure exhibited a significantly reduced immune response profile as compared to the benchmark LNP composition as shown by the reduced C3a serum levels.

B. Toxicity Profile

In another assessment, the levels of four liver enzymes present in serum were evaluated at 4 hours and 24 hours after LNP administration as a measure of potential hepatotoxicity. LNP compositions were prepared as described in Example 9 with the following mole percentages shown in Table 29. The LNP compositions encapsulated 5′-CleanCap-5MeC-fLuciferase mRNA (TriLink Biotech) as described in Example 9.

TABLE 29

| | | | | | Total | |
|---|---|---|---|---|---|---|
| LNP | ssPalmO—Ph—P4C2 | Phospholipid | Chol | PEG-DMG | Lipid | Lipid:RNA |
| ID | (% moles) | (% moles) | (% moles) | (% moles) | (mM) | (w/w) |
| 5.0 | 54 | 10% DOPE | 35 | 1 | 25 | 100 |
| 5.1 | 54 | 10% DOPE | 35 | 1 | 25 | 75 |
| 5.2 | 54 | 10% DOPC | 35 | 1 | 25 | 100 |
| 5.4 | 54 | 10% DOPC | 35 | 1 | 25 | 75 |
| 5.5 | 59 | 5% DOPC | 35 | 1 | 25 | 100 |
| 5.6 | 59 | 5% DOPC | 35 | 1 | 25 | 75 |

Briefly, blood was drawn at 4 hours and at 24 hours and each sample was allowed to clot for 20 minutes and subject to centrifugation at 13K rpms for 3 minutes to remove undesired cells and debris. The samples were placed on wet ice for transport, and store at −80° C. until analyzed. Enzyme levels were determined using standardized assays (IDEXX)

The levels of the liver enzymes aspartate transaminase (AST), alanine transaminase (ALT), alkaline phosphatase (ALP), and creatine kinase (CK) at 24 hours are shown in Tables 30 a-d, respectively. The level of each liver enzyme in the samples treated with the LNP compositions of the present disclosure was compared to the level of each liver enzyme in the samples treated with an LNP composition comprising a benchmark phosphoethanolamine (PE)-based phospholipid and the values are reported in Tables 30a-d.

TABLE 30a
AST Levels

| LNP ID | AST Amount (Units/L) |
|---|---|
| 5.0 | 193.25 |
| 5.1 | 346.25 |
| 5.2 | 115.0 |
| 5.4 | 110.75 |
| 5.5 | 270.5 |
| 5.6 | 238.25 |

TABLE 30b
ALT Levels

| LNP ID | ALT Amount (Units/L) |
|---|---|
| 5.0 | 120.5 |
| 5.1 | 186.25 |
| 5.2 | 60.25 |
| 5.4 | 70.75 |
| 5.5 | 195.25 |
| 5.6 | 164.5 |

TABLE 30c
ALP Levels

| LNP ID | ALP Amount (Units/L) |
|---|---|
| 5.0 | 70.75 |
| 5.1 | 63.75 |
| 5.2 | 70.0 |
| 5.4 | 62.0 |
| 5.5 | 63.0 |
| 5.6 | 64.25 |

TABLE 30d
CK Levels

| LNP ID | CK Amount (Units/L) |
|---|---|
| 5.0 | 361.75 |
| 5.1 | 139.0 |
| 5.2 | 85.75 |
| 5.4 | 163.75 |
| 5.5 | 105.5 |
| 5.6 | 116.25 |

As shown in Tables 30a-d, specific LNP compositions of the present disclosure exhibited a significantly reduced toxicity profile as compared to the benchmark LNP compositions as shown by the reduced serum liver enzyme levels.

### Example 11—In Vivo LNP-Delivery of DNA to Liver

The following is a nonlimiting example demonstrating that LNP compositions of the present disclosure can be used to deliver DNA to liver cells in vivo and expression of the encoded protein.

LNP compositions of the present disclosure comprising DNA encoding firefly luciferase (hereafter “Fluc”; TriLink) were prepared as described in Example 9 by combining ssPalmO-Ph-P4C2, the phospholipid DOPC, the structural lipid cholesterol (Chol) and 1,2-dimyristoyl-sn-glycerol methoxypolyethylene glycol (DMG-PEG2000) in the mole percentages presented in Table 31,

TABLE 31

| | | | | | Lipid | |
|---|---|---|---|---|---|---|
| LNP | | | | DMG- | Blend | Lipid:DNA |
| ID | ssPalmO—Ph—P4C2 | DOPC | Cholesterol | PEG2000 | (mM) | (w/w) |
| D6 | 54 | 10 | 35 | 1 | 25 | 100:1 |

Adult BALB/C mice were administered 0.5 mg/kg of total DNA (n=4) of the LNP composition listed in Table 31. The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 32.

TABLE 32

| | Mean Liver Luciferase |
|---|---|
| LNP ID | Flux (p/s) |
| D6 | 1.35E+07 |

As shown in Table 32, LNP compositions of the present disclosure were capable of delivering DNA to liver cells and express the encoded transgene in liver cells.

### Example 12—LNPs of the Present Disclosure for the Treatment of Hemophilia

The following is a non-limiting example demonstrating the compositions and methods of the present disclosure can be used to in the treatment of hemophilia, and more specifically hemophilia A.

Neonatal, wild-type BALB/c mice (n=5-6) were administered 0.25 mg/kg FVIII transposon LNPs co-delivered with either 1) 1 mg/kg of LNPs encapsulating functional SPB (“Functional SPB”) or 2) 0.5 mg/kg LNPs encapsulating catalytically deficient SPB (“Deficient SPB”).

The FVIII transposon LNPs were ssPalmO-Ph-P4C2-containing LNPs of the present disclosure comprising nanoplasmid DNA comprising a transposon, wherein the transposon comprised an expression cassette comprising, a first piggyBac inverted terminal repeat (right ITR), followed by a first insulator sequence, followed by three tandem copies of the SERPINA1 enhancer, followed by a Transthyretin (TTR) enhance/promoter and minute virus of mice (MVM) intron sequence, followed by a codon optimized nucleic acid sequence encoding for modified human Factor VIII (FVIII), followed by the AES untranslated region (UTR), followed by the mTRNR1 UTR, followed by an SV40-late polyadenylation and cleavage signal sequence, followed by a second insulator sequence, followed by a second piggyBac inverted terminal repeat (left ITR). The sequence of the Transposon is put forth in SEQ ID NO: 34. The FVIII transposon LNPs comprised ssPalmO-Ph-P4C2, DOPE, Cholesterol and DMG-PEG2000 at a molar ratio of 54:10:35:1 and had a lipid:DNA ratio of 100:1 (w/w).

The functional SPB LNPs were ssPalmO-Ph-P4C2-containing LNPs of the present disclosure comprising mRNA encoding active SPB, and ssPalmO-Ph-P4C2, DOPE, Cholesterol and DMG-PEG2000 at a molar ratio of 54:10:35:1 and lipid:RNA ratio of 100:1 (w/w). The deficient SPB LNPs were C12-200-containing LNPs of the present disclosure comprising mRNA encoding catalytically deficient SPB, and C12-200, DOPE, Cholesterol and DMG-PEG2000 at a molar ratio of 33.5:32:33.5:1. All cytidine residues in the mRNA encoding either functional SPB or deficient SPB were 5-methylcytidine (5-MeC).

Prior to administration of LNPs, the neonatal pups were placed on ice for a brief time (approximately 3 minutes) to induce anesthesia. For co-delivery administration, both DNA-LNP and mRNA-LNPs were mixed together in a tube, 304 drawn into a single 29 gauge insulin syringe, and delivered via intravenous (IV) through the facial vein. Pups were brought back to normal body temperature on a 37° C. heat pad before being placed back with their mother. On weeks 2, 4, 6, and 8 post-treatment, plasma was collected from treated mice. For plasma collection, treated mice were put under anesthesia with isoflurane, approximately 1504 whole blood was retro-orbitally collected, whole blood was mixed with 10% volume of 3.2% sodium citrate, centrifuged at 15,000 g for 15 minutes at 20° C., and plasma supernatant was collected. hFVIII antigen levels were measured using the Visualize™ Factor VIII Antigen Plus Kit (Affinity Biologicals™ Inc.).

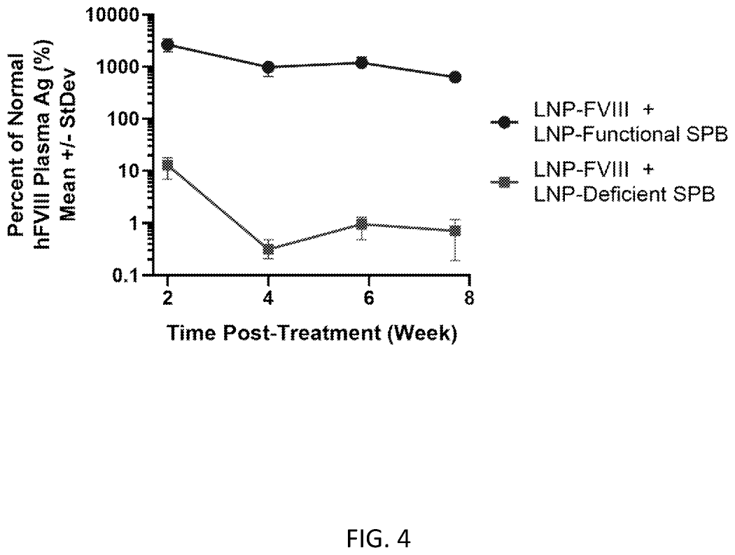

The results of this analysis are shown in FIG. 4. FIG. 4 shows that supra-therapeutic human FVIII protein levels were observed in the samples up to 8 weeks following administration of the SPB LNPs.

The results presented in this example demonstrate that the LNPs of the present disclosure can be used to drive high levels of FVIII expression in vivo, thereby demonstrating that the composition and methods of the present disclosure can be used to treat hemophilia A.

### Example 13—LNP Compositions of Present Disclosure Deliver RNA with High Specificity to the Liver In Vivo

This experiment shows the ability of LNP compositions of the present disclosure to deliver Cas-CLOVER mRNA to the liver, targeted by a pair of gRNAs to the psk9 gene, resulting in subsequent in vivo gene editing of the psk9 gene. Pcsk9 protein is secreted by hepatocytes and binds to the LDL receptor, inducing its internalization and lysosomal degradation, resulting in increased circulating levels of LDL-cholesterol.

In this experiment, each group of adult female BALB/C mice (n=2/group) was intravenously co-administered mRNA encoding 5′-CleanCap-5MeC-Cas-CLOVER (SEQ ID NO: 31) and a pair of gRNAs (SEQ ID NOs: 29-30) targeted to the first exon of the mouse pcsk9 gene. The mRNA and gRNA molecules were formulated within LNP compositions of the present disclosure comprising ssPalmO-Ph-P4C2, DOPE, Cholesterol and DMG-PEG2000 at a molar ratio of 54:10:35:1 (referred to as LNP Composition 6.1, 6.2, 6.3, 6.4 and 6.5 in Tables 33-36). All cytidine residues in the mRNA were 5-methylcytidine (5-MeC).

LNP compositions of the present disclosure comprising the total RNA doses shown in Table 33 were administered to the mice from each group. One group of mice was administered a dose of Cas-CLOVER mRNA and a pair of pcsk9 gRNA, both co-encapsulated in a benchmark C12-200 LNP composition. One group of mice was treated with vehicle (PBS, Thermo Fisher Scientific, USA) as a negative control.

TABLE 33

| LNP | Total RNA (mRNA and | mRNA/gRNA |
|---|---|---|
| Composition | gRNA) dose (mg/kg) | ratio |
| C12-200 | 1 | 1:1 |
| 6.1 | 0.5 | 1:1 |
| 6.2 | 1 | 1:1 |
| 6.3 | 1.5 | 1:1 |
| 6.4 | 2 | 1:1 |
| 6.5 | 3 | 1:1 |

After seven days post-administration, DNA was isolated from four tissue types from the mice in each group: liver, spleen, lung, and kidney. Briefly, tissues were resected after euthanasia, flash frozen in liquid nitrogen, mixed with lysis buffer (15 mg of tissue in 200 uL of lysis buffer+10 uL Proteinase K) and pulverized in a TissueLyser II (Qiagen) using Triple-Pure zirconium beads (Fisher Scientific). Homogenized tissue was then incubated at 56 C for 30 minutes, and column-purified using a Monarch Genomic DNA Purification kit from New England Biolabs under manufacturer's instructions. Final DNA elution was done in 50 uL of elution buffer (10 mM Tris-Cl, pH 8.5). Concentration and purity of DNA samples was assessed by measuring absorbance at 260 and 280 nm using a Nanodrop. Also, blood samples were drawn for LDL-C quantification. Briefly, 500 uL of blood was collected after euthanasia via cardiac puncture using 2 ml syringes and 25G needles, transferred to microcentrifuge tubes, incubated at room temperature for 1 hour, and centrifuged at 1500 g for 15 minutes to separate the cellular fraction from serum. Serum fraction (200 uL) was transferred to a new tube and stored at −80C until further analysis.

In addition, the body weight of mice treated with the LNP compositions of Table 33 was assessed during the seven days post-administration and the body weights at baseline and post-treatment were compared. The average percentage of body weight change after seven days post-treatment for each group of mice treated with each LNP composition of Table 33 is shown in Table 34.

TABLE 34

| | % Body Weight |
|---|---|
| LNP ID | Change 7 days |
| Benchmark C12-200 LNP | 3.25% |
| 6.1 | 4.11% |
| 6.2 | 0.51% |
| 6.3 | 4.38% |
| 6.4 | 3.09% |
| 6.5 | 3.86% |

As shown in Table 34, the LNP compositions of the present disclosure were well tolerated with most treated mice retaining original body weights or even slightly gaining weight.

Gene editing by the Cas-CLOVER mRNA delivered to the mice was measured by Next Generation Sequence (NGS). Briefly, genomic DNA samples were first amplified with primers flanking Pcsk9 exon 1 and containing Illumina partial adapters. The resulting amplicons underwent a second PCR reaction with primers containing Illumina P5 and P7 sequences and a unique index sequence (New England Biolabs). The final amplicons were pooled at equimolar concentrations, loaded in a Miseq Micro Kit v2 300-cycles (Illumina), and run in a Miseq benchtop sequencer following standard Illumina procedures for Amplicon-seq. Sequencing data was then analyzed using CRISPResso2 to determine the frequency of insertions/deletions (indels) in each sample. Results of NGS are provided in Table 35 as indel percentages found in the pcsk9 gene.

TABLE 35

| LNP ID | Pcsk9 indel % |
|---|---|
| C12-200 | 45.11% |
| 6.1 | 12.38% |
| 6.2 | 20.66% |
| 6.3 | 49.19% |
| 6.4 | 63.27% |
| 6.5 | 63.78% |

As shown in Table 35, LNP compositions of the present disclosure successfully delivered Cas-CLOVER mRNA to the liver as shown by subsequent gene editing of the pcsk9 gene, with indel rates at or better than the benchmark C12-200 composition for total RNA doses of 1.5 mg/kg or higher.

Serum levels of the pcsk9 protein in the mice were also measured 7 days after administration and the results are shown in Table 36. Briefly, a mouse Pcsk9 ELISA kit (Biolegend) was used to determine Pcsk9 in each serum sample following manufacturer's instructions. All serum samples were assayed in triplicate and results were expressed as percentage reduction in Pcsk9 levels compared with Pcsk9 levels of PBS-treated mice.

TABLE 36

| LNP ID | Pcsk9 reduction |
|---|---|
| C12-200 | 78.23% |
| 6.1 | 11.47% |
| 6.2 | 38.73% |
| 6.3 | 75.96% |
| 6.4 | 82.66% |
| 6.5 | 81.09% |

Taken together, the results of Tables 35 and 36 show that Cas-CLOVER mRNA delivered by LNP compositions of the present disclosure is effective at editing the pcsk9 gene in the liver in vivo. Gene editing efficacy is maximal at 2 mg/kg total RNA as shown by high indel % rate compared to benchmark and low pcsk9 protein expression levels compared to baseline.

### Example 14—LNPs of the Present Disclosure Deliver mRNA to Non-Human Primates

The following is a non-limiting example demonstrating that the compositions of the present disclosure can be used to deliver mRNA to Non-Human Primates (NHPs) in vivo and the delivered mRNA was well tolerated in the NHPs.

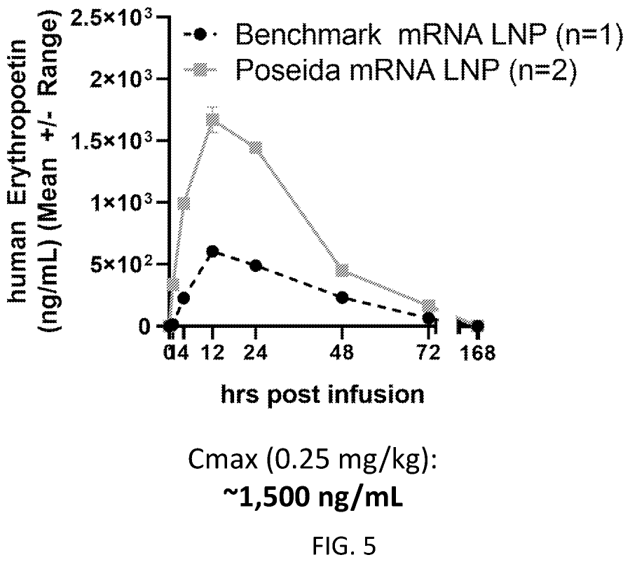

In the first study, mRNA molecules comprising a sequence encoding human erythropoietin (hEPO) protein were encapsulated in lipid nanoparticles of the present disclosure comprising about 54% of ssPalmO-Ph-P4C2 SS-OP by moles, about 35% of cholesterol by moles, about 10% of DOPE by moles, and about 1% of DMG-PEG2000 by moles (referred to as Poseida mRNA LNP in FIG. 5).

LNPs of the present disclosure encapsulating hEPO mRNA were administered to two monkeys and a benchmark lipid nanoparticle composition, MC3, was administered to one monkey. The monkeys were administered ascending doses of the LNPs with a 0.25 mg/kg dose administered on day 1 and a 0.5 mg/kg dose administered on day 21. Blood was sampled for one week post each infusion at the following times: 0 hour, 1 hour, 4 hours, 12 hours, 24 hours, 48 hours, 72 hours, and 168 hours. All blood draws were non-fasted and volumes collected were 2 mL each.

The levels of human erythropoietin (hEPO) in the drawn blood samples after the day 1 administration (0.25 mg/kg dose) were determined using standardized assays. Briefly, whole blood was processed to serum with standardized methods. hEPO was detected in the serum with an ELISA kit (R&D Systems) that was optimized for NHPs. FIG. 5 shows that hEPO levels in the samples from monkeys treated with LNPs of the present disclosure were higher than hEPO levels in the samples from the monkey treated with the benchmark composition over the 168 hours post-infusion. The peak EPO level following administration of the LNPs of the present disclosure was reached about 12 hours post infusion as shown in FIG. 5; also shown in FIG. 5, the peak EPO level was as high as 3× the peak hEPO level following administration of the benchmark LNP over the course of the 168 hours.

The results of this study show that mRNA was delivered to NHPs in vivo as demonstrated by the increased levels of hEPO measured in the serum.

In another study, the levels of two liver enzymes were measured in the serum of rats and NHPs treated with LNPs of the present disclosure as a measure of potential hepatoxicity. In the experiments for this study, 5MeC-mRNA molecules comprising a sequence encoding HA-tagged SPB were encapsulated in lipid nanoparticles of the present disclosure comprising about 54% of ssPalmO-Ph-P4C2 SS-OP by moles, about 35% of cholesterol by moles, about 10% of DOPE by moles, and about 1% of DMG-PEG2000 by moles.

In the first experiment, the levels of the liver enzymes aspartate transaminase (AST) and alanine transaminase (ALT) present in the serum of adult rats were evaluated at 4 hours, 24 hours and 7 days after administration of LNPs of the present disclosure at concentrations of 0, 0.25, 0.5 and 1 mg/kg. Rats (n=3) were injected with LNPs of the present disclosure or vehicle (PBS) intravenously via the tail vein and blood was drawn at 4 hours, 24 hours and at 7 days.

In a separate experiment, the levels of AST and ALT present in the serum of female cynomolgus monkeys were evaluated at 0 hours, 24 hours and 7 days after administration of LNPs of the present disclosure at concentrations of 0, 0.1 and 0.25 mg/kg. Monkeys (n=2-3) were injected with LNPs of the present disclosure or vehicle (PBS) and blood was drawn at 0 hours, 24 hours and at 7 days.

After serum samples were drawn, each sample was allowed to clot for 20 minutes and subject to centrifugation at 13K rpms for 3 minutes to remove undesired cells and debris. The samples were placed on wet ice for transport, and store at −80° C. until analyzed. Enzyme levels were determined using standardized assays.

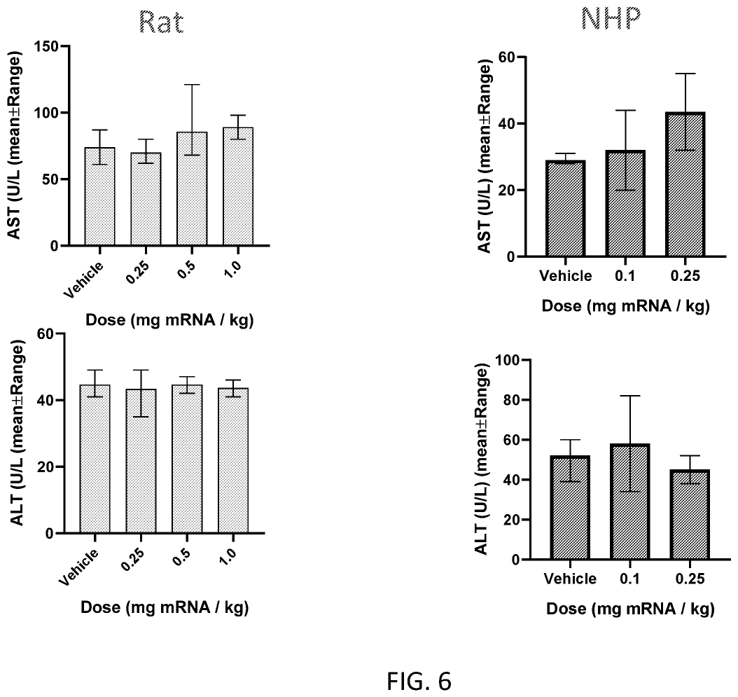

The levels for AST and ALT after 7 days in the test subjects from the two different experiments in this study are shown in FIG. 6. As shown in FIG. 6, the in vivo administration of the LNP compositions of the present disclosure resulted in no meaningful increases in AST and ALT levels in serum after 7 days in both rats and NHPs.

### Example 15—LNPs of the Present Disclosure for the Treatment of Ornithine Transcarbamylase (OTC) Deficiency

The following is a non-limiting example demonstrating the compositions and methods of the present disclosure can be used in the treatment of OTC Deficiency.

1 day old B6EiC3Sn male OTCD pups (n=4-9) were administered the following treatments:

Treatment #1: Human OTC (hOTC) transposon AAV viral vector particles

Treatment #2: Human OTC (hOTC) transposon AAV viral vector particles in combination with SPB LNPs.

The Human OTC (hOTC) transposon AAV viral vector particles were AAV viral vector particles comprising a piggyBac transposon, wherein the piggyBac transposon comprised a nucleic acid encoding for a human OTC polypeptide.

The SPB LNPs were ssPalmO-Ph-P4C2-containing LNPs of the present disclosure comprising mRNA encoding active SPB. The SPB LNPs comprised ssPalmO-Ph-P4C2, DOPE, Cholesterol and DMG-PEG2000 at a molar ratio of 54:10:35:1 and had a lipid:RNA ratio 100:1 (w/w).

A separate AAV8 viral vector particle comprising a sequence encoding shRNA targeting endogenous mouse OTC was utilized to induce severe disease in the OTCD model by removing residual endogenous mouse OTC; in this model, only successful gene therapy with a human OTC transgene can rescue severe OTCD.

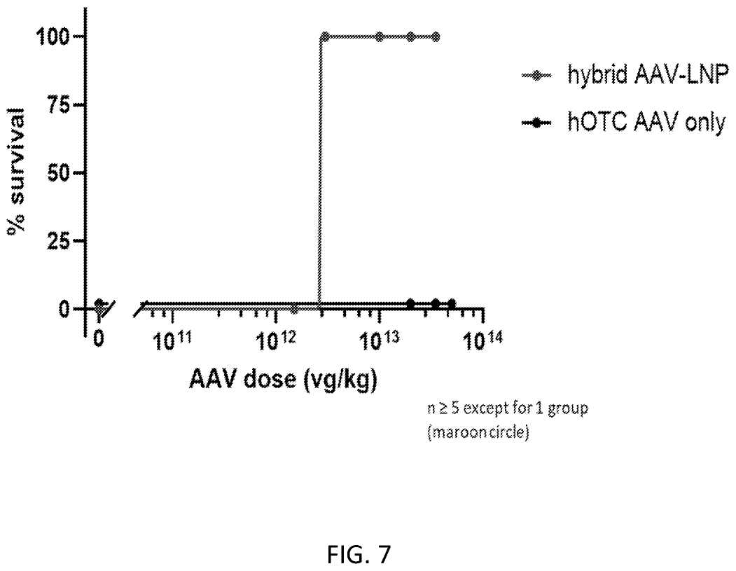

For both treatments, mice were administered increasing doses of hOTC transposon AAV viral vector; mice in Treatment #2 group were also administered 0.5 mg/kg SPB LNPs. FIG. 7 shows that 47 days following administration of the treatments, 100% survival of OTC-deficient mice was observed in Treatment #2 group in the low dose range of AAV, as compared to treatment of AAV alone which resulted in severe OTCD morbidity across the entire dose range upon severe OTC disease induction.

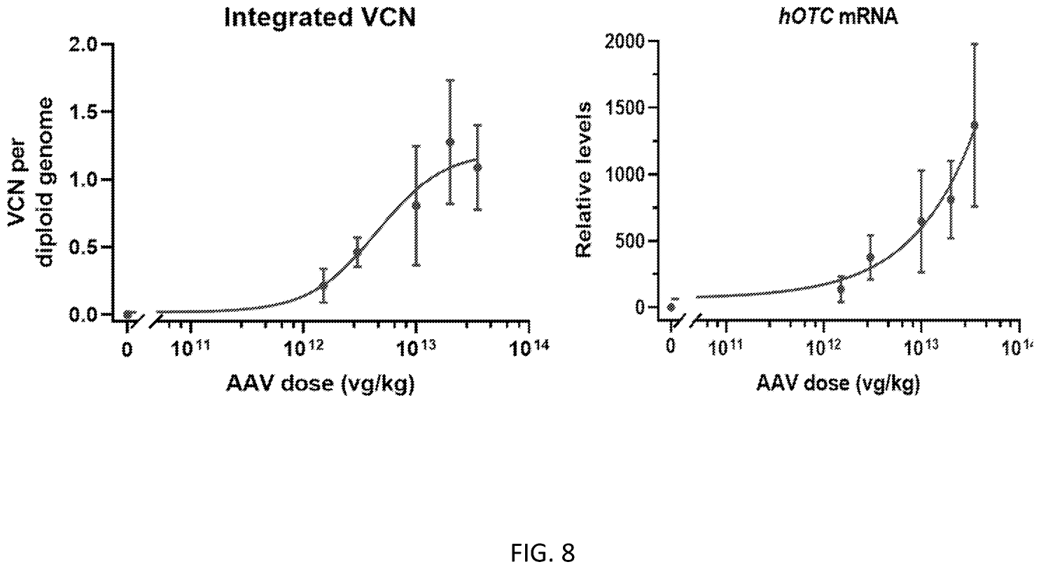

Further, the levels of integrated viral copy number (VCN) and hOTC mRNA in the liver of mice in Treatment #2 group were measured by droplet digital PCR (ddPCR) and real-time qPCR, respectively. Briefly, DNA was isolated from powdered liver tissues using the DNA Rapidlyse kit (Macherey-Nagel), and the vector copies were analyzed with the QX200 Auto DG droplet digital PCR system (Biorad) utilizing separate primers targeting the 3′ UTR of the hOTC transgene and the AAV backbone outside of the piggyBac ITRs to differentiate episomal and integrated vector copies. Primers against the HMBS (hydroxymethylbilane synthase) gene were utilized for normalizing the number of mouse cells. For mRNA quantification, mRNA was isolated from powdered liver tissue with the RNeasy mini kit (Qiagen) and after conversion to cDNA (high capacity RNA to cDNA kit, ThermoFisher), gene expression was analyzed by real-time quantitative qPCR (Applied Biosystems QuantStudio 6) using specific primer sets targeting hOTC and mActb (beta-actin). FIG. 8 shows an AAV dose dependent increase in both integrated VCN and hOTC mRNA levels, demonstrating that administration of hOTC transposon AAV viral vector particles in combination with SPB LNPs of the present disclosure successfully integrated functional OTC genes into the liver.

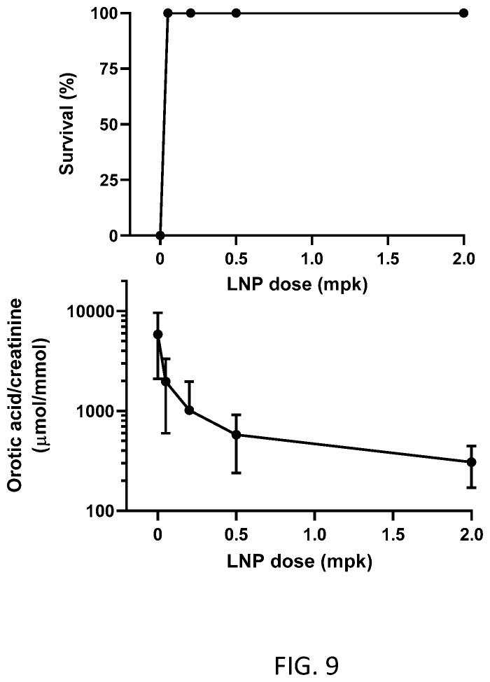

In another study, the level of orotic acid, a biomarker that is increased in OTCD, was measured in the urine of treated mice. Briefly, 1 day old B6EiC3Sn male pups (genotype confirmation only at 21 days of age) (n=3 for lowest dose group, 6-8 for remaining groups) were administered a 2E13 vg/kg dose of hOTC transposon AAV viral vector particles in combination with increasing doses of SPB LNPs. Orotic acid levels were measured by liquid chromatography tandem mass spectrometry (LC-MS/MS). Briefly, diluted urine samples were analyzed with a reverse phase UPLC column followed by MS/MS detection with a Micromass Quattro in negative ionization mode. Results were normalized by measuring creatinine levels in the urine with hydrophilic interaction LC-MS/MS and data were collected in positive ionization mode. FIG. 9 shows that 47 days following administration of the treatments, 100% survival of mice was observed across the entire dose range of SPB LNPs along with a dose dependent decrease in orotic acid upon severe OTC disease induction.

The results presented in this example demonstrate that the LNPs of the present disclosure can be used to drive high levels of human OTC polypeptide expression in vivo, thereby demonstrating that the composition and methods of the present disclosure can be used to treat OTCD. Moreover, the LNPs of the present disclosure can be used to successfully integrate OTC genes into liver and resolve disease phenotype, as measured by orotic acid.

What is claimed is:

1. A composition comprising at least one lipid nanoparticle comprising between about 53% to about 60% of ssPalmO-Ph-P4C2 by moles;
between about 34% to about 41% of cholesterol by moles,
between about 4% to about 11% of DOPC, DSPC or DOPE by moles, and
between about 0.5% to about 2% of DMG-PEG2000 by moles,
wherein the at least one lipid nanoparticle comprises at least one nucleic acid molecule, and wherein the ratio of lipid to nucleic acid in the at least one nanoparticle is about 70:1 to about 105:1 (w/w).

2. The composition of claim 1, comprising at least one lipid nanoparticle comprising about 54% of ssPalmO-Ph-P4C2 by moles;
about 35% of cholesterol by moles,
about 10% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).

3. The composition of claim 1, comprising at least one lipid nanoparticle comprising about 54% of ssPalmO-Ph-P4C2 by moles;
about 35% of cholesterol by moles,
about 10% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 75:1 (w/w).

4. The composition of claim 1, comprising at least one lipid nanoparticle comprising about 54% of ssPalmO-Ph-P4C2 by moles;
about 40% of cholesterol by moles,
about 5% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).

5. The composition of claim 1, comprising at least one lipid nanoparticle comprising about 56.5% of ssPalmO-Ph-P4C2 by moles;
about 37.5% of cholesterol by moles,
about 5% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).

6. The composition of claim 1, comprising at least one lipid nanoparticle comprising about 59% of ssPalmO-Ph-P4C2 by moles;
about 35% of cholesterol by moles,
about 5% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).

7. The composition of claim 1, comprising at least one lipid nanoparticle comprising about 59% of ssPalmO-Ph-P4C2 by moles;
about 35% of cholesterol by moles,
about 5% of DOPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 75:1 (w/w).

8. The composition of claim 1, comprising at least one lipid nanoparticle comprising about 54% of ssPalmO-Ph-P4C2 by moles;
about 35% of cholesterol by moles,
about 10% of DSPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).

9. The composition of claim 1, comprising at least one lipid nanoparticle comprising about 54% of ssPalmO-Ph-P4C2 by moles;
about 40% of cholesterol by moles,
about 5% of DSPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).

10. The composition of claim 1, comprising at least one lipid nanoparticle comprising about 56.5% of ssPalmO-Ph-P4C2 by moles;
about 37.5% of cholesterol by moles,
about 5% of DSPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).

11. The composition of claim 1, comprising at least one lipid nanoparticle comprising about 59% of ssPalmO-Ph-P4C2 by moles;
about 35% of cholesterol by moles,
about 5% of DSPC by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).

12. The composition of claim 1, comprising at least one lipid nanoparticle comprising about 54% of ssPalmO-Ph-P4C2 by moles;
about 35% of cholesterol by moles,
about 10% of DOPE by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 100:1 (w/w).

13. The composition of claim 1, comprising at least one lipid nanoparticle comprising about 54% of ssPalmO-Ph-P4C2 by moles;
about 35% of cholesterol by moles,
about 10% of DOPE by moles, and
about 1% of DMG-PEG2000 by moles,
wherein the at least one lipid nanoparticle comprises at least one nucleic acid molecule, wherein the at least one nucleic acid molecule comprises at least one RNA molecule, and wherein the ratio of lipid to nucleic acid in the at least one nanoparticle is about 75:1 (w/w).

14. The composition of claim 1, comprising at least one lipid nanoparticle comprising about 54% of ssPalmO-Ph-P4C2 by moles;
about 35% of cholesterol by moles,
about 10% of DOPE by moles, and
about 1% of DMG-PEG2000 by moles,
wherein the at least one lipid nanoparticle comprises at least one nucleic acid molecule, wherein the at least one nucleic acid molecule comprises at least one DNA molecule, and wherein the ratio of lipid to nucleic acid in the at least one nanoparticle is ab out 100:1 (w/w).

15. The composition of any of claims 1-13, wherein the RNA molecule is an mRNA molecule, preferably wherein the mRNA molecule further comprises a 5′-CAP.

16. The composition of claim 1-13 or 15, wherein the at least one RNA molecule comprises a nucleic acid sequence encoding at least one transposase, preferably wherein the transposase is a piggyBac™ (PB) transposase, a piggyBac-like (PBL) transposase, a Super piggyBac™ (SPB) transposase polypeptide, a Sleeping Beauty transposase, a Hyperactive Sleeping Beauty (SB100×) transposase, a helitron transposase, a Tol2 transposase, a TcBuster transposase or a mutant TcBuster transposase.

17. The composition of claim 14, wherein the DNA molecule is a circular DNA molecule, DoggyBone DNA molecule, a DNA plasmid, a DNA nanoplasmid, or a linearized DNA molecule, preferably wherein the DNA molecule is a DoggyBone DNA molecule or a DNA nanoplasmid.

18. The composition of claim 14 or claim 17, wherein the at least one DNA molecule comprises a nucleic acid sequence encoding at least one transposon.

19. The composition of any of the preceding claims, wherein the at least one nucleic acid molecule comprises a nucleic acid sequence encoding at least one therapeutic protein.

20. The composition of any one of claims 14, 17 and 18, wherein the at least one nucleic acid molecule comprises a nucleic acid sequence encoding at least one transposon, wherein the transposon comprises a nucleic acid sequence encoding at least one therapeutic protein.

21. The composition of claim 19 or claim 20, wherein the at least one therapeutic protein is ornithine transcarbamylase (OTC), methylmalonyl-CoA mutase (MUT1), Factor VIII or Factor IX.

22. A method of delivering at least one nucleic acid to at least one cell comprising contacting the at least one cell with at least one composition of any of the preceding claims.

23. A method of genetically modifying at least one cell comprising contacting the at least one cell with at least one composition of any of the preceding claims.

24. The methods of claim 22 or claim 23, wherein the at least one cell is at least one liver cell, preferably wherein the at least one liver cell is a hepatocyte, a hepatic stellate cell, Kupffer cell or liver sinusoidal endothelial cell.

25. A method of treating at least one disease or disorder in a subject in need thereof comprising administering to the subject at least one therapeutically effective amount of at least one composition of any one of claims 1-21.

26. The method of claim 25, wherein the at least one disease or disorder is a liver disease or disorder, preferably wherein the liver disease or disorder is a metabolic liver disorder, preferably wherein the metabolic liver disorder is:
i) a urea cycle disorder; or
ii) N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome), progressive familia intrahepatic cholestasis type 1 (PFIC1), progressive familia intrahepatic cholestasis type 1 (PFIC2), progressive familia intrahepatic cholestasis type 1 (PFIC3) or any combination thereof.

27. The method of claim 25, wherein the at least one disease or disorder is a hemophilia disease, preferably wherein the hemophilia disease is hemophilia A, hemophilia B, hemophilia C or any combination thereof.

28. The composition of any one of claims 1-13, 15 and 16, wherein the at least one RNA molecule comprises a nucleic acid sequence encoding a fusion protein, wherein the fusion protein comprises (i) an inactivated Uas9 (dCas9) protein or an inactivated nuclease domain thereof, (ii) a Clo051 protein or a nuclease domain thereof.

29. The composition of claim 28, wherein the composition further comprises at least one guide RNA molecule.
